SEQUENCE LISTING (29 sequences)

SEQ ID NO: 1 - DNA, 538 bp, Artificial Sequence
  promoter sequence
ggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatc

SEQ ID NO: 2 - DNA, 2000 bp, Artificial Sequence
  promoter sequence
cccacttgctccctgggcttggagcacaaccactcaaacagaactggcttttggtcagtaaggaagaagtgagcaacggctgcggtgtagaccctcgtcaatgcctgcgacggttacacctggagacaagctccccagtgtcctcaggagcagcggagatgagaatccatgatagggtgggctctgtccccctcagctccgtgatgccgaaatgcactgctggtcctggtcctgctcctcattccacacccggctgagtgcccatctgaccccagacctcaacgcgaggttctaagcactgtctcctgacccttcaaccccttcgggattttgcatgtgctgttggaccacctcactcccacctggagccaaatgacactgtaggaggaggggaagagaacttatgctagtagagtgtgtgtgtgtgtgtgtgtgtgtgtgtgaatgtgtgtgtgtattatacataatatatataattacaacattgttaatggggccgggcgcggtggctcacacctgtaatcacagcactttgggaggctgagatgggcagatcacatgaggtcaggagttcaagaccagcctggccaacatggtgaaaccccgtctctactaaaatacaaaaattagccgggcatggtggcgtgcgcctgtagtcccggctactcagcaggctgaggcaagagaattgcttgagcctgggaggcggaggttgcagtgagccaagattgcaccactgcactccagcctgggcaacagagtacgactccatctccacacacacatacacacacacacacacacacacaaattgttaattgtatatgtataattataatagtttatatatattaacaattatatataaacttgcatatatatagtatttgctgtattattatatataaacaattatatatgtaatgactgtataaaatagataaacaattttaactaataatattatattaattatattattattagatgtaataattataattatttatatataatatttattattatgtatcattgttagaacacttaacgtgagctctgtcctcttaacaaatttcaagtgaacaagacgttattgctgacgatgggtcgtatgtggtgcagcagatctctaggcctgtttgttaataactccccatttccccctcctcccagcccccgtaaccaccattccctgctgtgatgttgtgactctggtgactttgcagatctcctgtaagtgacatcatgcagtacttggtctctgcctctgcgtcgcttggcgtgatgtcctcaggtttcgtccgtgttgtcgcccatggcagaattttcttccttgtttaaggctgaatagtattcccctgtgtgtgcaccacattttctatatcaattcttctatcaatggacatttagatgattttcacgtcttagctattgcgaatagtgctgcagtgatcaggggagttcagacggctctttgcatactgaatttgtttcttttaaatatagacccagaagtggcattgttggaccatacggtagctctatgtttagttttttgaggaacctccccactgttctctatagtgagtgcacaatttttcagcctcccaaagtgctgggatcacaggcgtgagccaccgcgcccggcgcactgtaggatcttttttaatgcattatataccttgctgagattttagcagagatcacaatattaaaaacttggggaaggatttctatgactctcattttaaatacaaggacatgtcgacttctagttttgtaacatcttgcccaagagcggtggtcgttaatgtgtggagttgggatgacatccaagtcagttggttgcacgacctttattctgtcttgtcccatagatttagaaagaggctgacacatcgggtaactagtttaaggtcatctgatcatgcgggtaagcgacatttttcagaaaccaaggccctccctctcatctcactagtgggaagggtggaaag

SEQ ID NO: 3 - DNA, 2000 bp, Artificial Sequence
  promoter sequence
ttttccactgcccttctcagccccacaggccttcaggaagacccgaacttcaagcaaagcctttttatttttcaacacccacagcttccattcaacaatcagaagttttccactttgatcaaagaggctgaacaagagggccaagggagcagctggttctcagcaactctgggcagaccacagagcccttgctacccactcactgtccgtgcccaccagaggacacagccttctccccaaatcagccaggtacatgccccagaaaacactggcttgcctcgttcccaccttaattaccggaccaaacgaacgtgaacacactgttttcaaaaccaaactcaattgggatcacgggggcctcggtttccttgattgtaaaatgggtatatcgccccccactctatttagcccaagaaaacaatttatctcttgatgtctctttcgtctccaggatctatgcttttacagactcactgggaggaaatatccagaccaaatcctaaagcctgatctaatttgggagatgctcagaagttttggttctatgcaagaacagcagtggtaataatccaagcttggctttagacacaggacgtttccttaggggcatctgggatctctgctggctcaaagtgatgcgctgcagacaaaaagtgagcagaaaggaaaggaggtgctatgcagaatgagcttcttccacggtgataccaaatggagctttcaaaggcccacatctggaggcagcagctatgcagtgattaacattttaaacggtattttgaaatggagatcattagtaaccacagatgtgatctgactctgtcccccaggtaatctgtctattgtatctaaattccagacttagcccagtagacagcttgggatgtttaacaggaattgtccaacaccatccccaaatctatttttattcatggagtactctgacatcatctcgcttggtcttcctgatgactgtaatgcagattgggaacagagaaagccataaagacttgtatgatggcccaaggctaagaagggaaagggctgagaccacacttcaggttttagctttcaggtgcagtgaagattgaatgacttagcacgagctttcagccaggcaggctgcaaagtgcacccaagttctttcagtgatcgacacttgcgacttaggttgagatagttttcatcctccctgagcctcagttttcccatctgtaaaaaataaacattgcctgcctcggagggtggaggtggaagtggaatgaagccacatgtaactcctagcgctgtgcctgagacagtagaggttcaattatagtagtcacatacacacacaacacatacatacacaagacacaacacaccacacacaacacatacatacacaccacacacaacacttacatacgcaccacactcggcatgcattgcagatcacaaatgcacaccacacacacactgcatacatataccatagagaacacacaacacctacacataaccccataccacaaatacacacacactacatagtacaccacgcagaacacacacagcccccatattccacaccacaccatctcactgccaattccttcccctcttcatgagttttacggcaggtccaggttcatctgccagtttaacagatcccaaaacttctgcaggagtgtttgttcaaaagattgatatttccagattcctgctttctgacagtatctttgaaccccaaatttcatactgccatgagccagtccccctttggagaaatatctccatttgtgtgccctttttcccccagggaacctgcagcatgtccctttttcagcagtagcctatcaaaccgaaccctttggagttattacactgcagtccgagggatccggcctccctgagacccagcaaggactcattatctggggaggtcttctgagccacaggcctcgctgaaagaaggtgcagcttcttgaacaggaaggcgttttgtggcagagtctaaa

SEQ ID NO: 4 - DNA, 2000 bp, Artificial Sequence
  promoter sequence
caggatagccctttcaatgcggaagtagatgaaagtttacccctacctcacaccatacacagaatgaattcaaatggatcatagagctaaatatgaagagcaaaagtagcaaaacttctagaacaaaacatagatgatcttcatcgatgttggattaacaacaatctcttgaacaggatgcagaaaactctaaccagagttcccacaaattgataagaaaagcaaacaccctagtgaaaaaaaaaaaagagcagaatatctgaacaggaacttccccagagaggatattcaaatgggcaataagcctgtgaaatggtgcccaatatcattatccctcagggaaatacaaatgggaaccacagggagatacaactacacaccacccccaccacagagtggctaaaattagggactgataatattaagtgtgcatgaggatatagagctttgtacaactgcttggcagttcctaatggagtttcacgtgctctgtgatccaacaaccccactcctagatgcacacctaacagaaatgcatgcacaaggcactcaaagacacatacaagaacgtcctcagcaacattagttacaatagcaaaacaaccgaaaacttcctgaatgcatatcaacagagaaatggataaatgaattatgttatatttattttatttatttatttttgagctggagtctcactctgtcccaggctggagtgcagtgatgcaatctttgctcactgtaacctccacctcctgggttcaaatgattctcctgcttcaccctcctgagtagctgggattacaggcatgcaccaccatgcccaactaatttttgtattttttagtagagacagggtttcaacatgttggccaggctggtctcgaactcctgacctcaggtgatccaccccccccttggcctcccaaagtcccaaagtgctgggattacaggcgtgagccaccacgcctggctggtatatttattaatcaaatactacacagcaataacagtgaactaactgtggctacaagcaataatgtggataaattttacaggcataatttgaggaaaaagaaatgagatatgatagagaatatgctatatggttctatttacataaggctcaaccacaggcacaattcatatacaagtgatacagcagtttactttctgggggagggaggagataactggaggaggctgagctgggggcagaggtgcatctgaggttctgagaatgttccagagcaggctatctccaccttggcactagtgaaattttgttggggagactgtcctgggcactgtgacattcccggcctctacccactagtgccggtaccacgatgtccaccccacccaccaccacccccgcctcattttggaaacaaaaatgtctccagacatttccaaatgtccccttgggtggaggaggtacaaaattcccctggttgccctgtttgattttgggtcgtgggggaattaggggtgtgggctcatttatggaaattccttgagctatacacatatgattcatcaacttttctgtacacatataaagcttttaaagtttttaaaataaaaaaacaagtcaatgaatgactgaaagaaccagtaagttttccagcaatccgagacaatagatagagtggggtttaatgtcttgttcacagacaaggagactgggatccagggtgggtcagggcagggggccagcaggatccaggagggaactcattccctgacttctcctccgcttccttcccccatgctctcacgcattcttgctcccgaatactagccaagtccctacagaggctgatcccgtgcaggggctcggttggacctttagcctcaaactcccgtggtggaaacagttaggattggtggacgctgcacccagcatcctgcaaacagaattctgaaaactgatatctcatgcaaacaggatatcaacttttgtcagaaagatcaaaaagagcttgaggtttttcttcaca

SEQ ID NO: 5 - DNA, 2000 bp, Artificial Sequence
  promoter sequence
catgtgcgtacagcaatgtctggcacataatcagtgcgactctcattattgtagtatgaatgtgatttgatgctgtaatttctcctctcttcagggtgccggtgtctttttctctcaatactccaagaaatgagcgtccagcatggatgtgaagggatgcttgctgcacctggagtttcaagatgccgctggggaaaagggaggctgctagcaaacaccattttggagggggctgtatgaaggagaaaagtagcctagagccggaactcagattttctaaaggggattcaggcggccagaggatagtagagggccagaaaggaggtccccaaggaggcttcggggtagggtgtgaggggatcctcaggcagactggggagctgagcctgttgtaaccgagcgagttacagagaaacgccgcactttgagacgaattcaggggtcctttattagctggcgactgagagacagctagtgctcaaaattctctcagccccaaagaaggggcttgattttcttttatactttggtttagaaaggacaggtgggggtctaaaacaatcttacagaagtaaagcaggcaaaaagttaaaaggataaatggttacgggaaagcaaacagttccaggtgcaggggcttaaaatctatcacaaggtgatagacacggggctttgggcgttatcaaccggacacaaacgccggggctctgggtgctattaaccgggcgaattcctgggaactgcggatatagcttgccacagtatcttatcagttaattgcattcttggatgtgctgggagtcagcttgcacaaattaagtccttgaggaagcggggtgggtaaggggctgcaaatgaaagagccaagatggagtctgtctggctctcttagctaagggagagtcaattcaggttaaaacaaggtagggtatcacaagcccacttaacaagggcagcaggaccccaagaagaaaagctttaggagtctccacagtgggcccagggcagttccacacaggtctctgaggccccacagacaggagagctgtgacgactcccttagtgcccaagaaagcaaggaggtggtgggcaaggggctcccagaggccttggggcactagaggggagatggagccgagggagtggctctgcaggcccctctctgagagagtgcatgaggtgtggccccaggcccaagggtagggggtgcacagggtgagggaagggaggagaggaagaggaggaggaggtggtggccacagcgggatggcactcagccaggttcagcttcgtgggaaaggtcccaggtgggccgggctagcactggggatgccctggctctgtgtctcggttgggtggctgactcctcctggaacgcatccctgacatcctccaggctggcttggggatactctggggacacacagtgcccaggcttccggcctcccagccctggctccctgtggactcagctgcccccaacaagcctgcgagccactggagagcaagggcaggctgtccttgccatccagatgcctggcacagaaggggttctcaccttcggggtatgggctgactcagtgggtttctcctccctccagctctaacctgcctgtggaataaacaaatcactcctctgggttgtctagctggtgcaggccccagggcgacgcccctttgaggggaaaatgggggcattccagtttccatgagccctgggactcccctccttcccaccactgctgcctaccctccggttccaggtatgcaggcttcctcccttctgacggttcctgctgctggagtcgtccttcctgaaaccctgcctttgcttagcctcattcccatctctcagtcccatcctgcagctgggcaggcagtgctgggccccggaaatgccctctgcctccctggagcacgtggcctgtgattttccttgagcacagcactttgtgactttgatgtaaacatcaaacacagccccctttcctgtcttcgca

SEQ ID NO: 6 - DNA, 2000 bp, Artificial Sequence
  promoter sequence
gcttcacatcgctagttctcagaaagagagcgcgagttgatctttcaaatttagagtccgggcgagtagggatggccagtttatgaatgtcttaaagaagtttattaaaggtgcgtgtgggcatattttttttaagccactttcctggaaagattttcccggtgagagggatggcagaagagggaggccctactagactttggaccacgtggaggtggggtgttgggggtgggcagccgggtgcagcctgcccggccagggcgaggaggtgcaagtccgcgccccgcccccggtctccatccgctcggcctcgcgtccgcccaccgtggagcccacagtttacagaactgcaagccagggcacattttattgttattattttttagggtatgggactggaagggatctctttctgttctcgctcacggactctgggcgctgttaaattgttggtgccatcgccccacccaagaccttcgggacgaaaacaaaaacccccgtgtgccccccgaggaaaaggccccgagggaggcggcgctgaggcgtcacggcctcgccatgcgcccgggtgaagccgccggagccaggcctgggggcggagtggggtgggtgcagctggactcgcccgcgaagcctagccggagccaggccccgacccgccctggccgcctccagagaaaccctgagaagggagaggaggccagccgcgtggcgcgcccagccctggaggccaggtcatcaaattgacaaagttggccacagcctggggaatggggggtgtccacgggggccgtggtctggccttgtccccgggccacggagaggccccacaactcctcttgtcctcgccggttggagatcaaacggctgacaaggcagctgcgtccttcccagtctgacaggcatttccaagtcctaccctgggccgaggagcgcggggggcggggtgtgacttccaggttgctgtgattgtcaggggcgcctcctggagtcacccacccgagtatctgcggagatttggccacacctactcctgggctggcaagtccaagtccctctcccacgtggagcgccccggtccctttaccctctcgcttgccatagacccagtttaggaggtggggtcgtgtttgaccccaggagttaatcggggtaaggtgaggggaggggacaaagatttgagtgaaaatccttttccaagtgggactcaaacaaggtctcaaacacttctcaccccctcacgtgctcttgattctgttgtcttagtgtcgtttattttgtcatcctcaccctcccagagtctgtaataccaggatttatatggagacactttttggaagttgaactccaacatctactcccctcccctcgccgcccgcgaccataaaacaaaagtaaaaactaccaagcgacttttccagggaaacgaagcgattcttactgtccccccaactcccatgtcgaccccaacccttttccacagtccacgcccccacccccaaagtccaagtccgaaagagcttttgggtgggtggagacttgcattgattcaatttagttcactcacacagcaccccgccccccttctcagggggtcctgctcccaggatggaggagatataaggacgatttttcttttattttaaaataagctgcccagtggccccccccaaccccctcccgctgttgcgcagccggggctcgggggagatgagcgcacaaaaacgcggtttgcacgtgtgtccggcttgggctgcgggtgtgcgcaactggcgactgtgtgtgtgtgtgtgtgagtgtgcgcgcgcgcgcgagtgtgtctctgtgtgtgctttcttgttctcttacagggtacaatgttaaaaagccaccgctagtcgcccccagtgctccgactctctgggtctttttgtctctagtgcagattaaacgtcacgtccgcacttgaacttgaattttatcccattgtacagaggcagccccagccatagagagaccga

SEQ ID NO: 7 - DNA, 2000 bp, Artificial Sequence
  promoter sequence
tagaaagctttccaaatcctggtgcctggcgtattccaatagtcttcttcccagtcttcctggttacatttctccctgaacccatcctccccatccctaaaatttcccccaaatgtgaactcagtcataccagtttgctccttatgtttcattggcccttgctgctaagagcatccgcttgcaccttctgctcatccccagacaagctttgtcctgtgaccataatgaactcttcatgccgtttccaactttagcccatgttattcttcttgtctgaatatccacccttttctctgttctcaataataagttcaggcttttcgtcttctgagaagccctttctgacttccacaggctgaaccactggcttctgctcctctacataatacttcaattccagcattgatctcactctatcatgatcatgggtttagctgtctgtccctgccactgctgtgtgttcctcttgagggcaggaacatttgtttttcactttttaaaaaacctctgttgcccagtctggcattaggaagtgcccattaggttgttattgcttgttggcgcttgagctggggcttgaaggtttctataatgtgtagcagtgtatagaaaacaggcaggtcagaaaaggcttctgtgcatcacaccaacatggcacatgtatacatatgtaacaaatctgcatgttgtgcacatgtaccctaaaacttaaagtataataataataaaattttaaaaaaaaaaagaagaggcttcctggaggagatgacagctgagctaagtcctggaggatgagaaggagtataaaataagataataggagaaaaaaggcagtaggaacagcatgggtaaaggtgatgaggcctgaaagaggcacgtggaaggaaagacaaatgcaggaagggggaatgggagggaatgctggggtacaggccaaagagggaggcatttggtgagtattctgcagagtctcctctgctgtgctgaggtgtggacaatgggaaaccatggacggactggagtaggcaaatgtcatattccctgttacaactgtctgtttgcatgtcagccttctagaagccccttaaggtatcaactatgtttttgttttgtcatcattcaatcctaagtgcacagaattccgggcatattacaggttccccatgaatgtttctttctttattaaaatgtatgaaaactctccagatttaaggaaggtcctcaatgtttcaaattctttttgttagatcattggtcctgtctacagctgtcacaaatttaaggactctggttatatttaatcttcacttttgaattttctgcttgaaaaatttgtattagaaaaaaaagtctatccttttatggacggctctaatctcttgaatcatttgggttggcttttctttggaccttcttcaactctgttttgtctctgttgagttaaggcttttaagaacacctgaattctttccttctgcaaaaccagaggcagcttcttttccgcctattttcagtttatttcttgtgattttagtttttttctcttaaccaaatgctaaatggatttaggagaaataaacttatttgtaaagctgtcaagggaccattagaaggatggtgcttcacagatagaatacagtttttattaatgatgcctagacaaatcctgccattagcccaagggctcagaaagttagcagcctagtagttttggagttgtcaatgaaatgaattggactggatggttaaggatgcccagaagattgaataaaattgggatttaggaggacccttgtactccaggaaattctccaagtctccacttagttatccagatcctcaaagtgaacatgaagcttcagtttcaaattgaatacattttccatccatggattggcttgttttgttcagttgagtgcttgaggttgtcttttcgacgtaacagctaaacccacggcttcctttctcgtaaaaccaaaacaaaaaggctttctattcaa

SEQ ID NO: 8 - DNA, 2000 bp, Artificial Sequence
  promoter sequence
tgaggcgtgagcgtctacagtgaacccagcacagaaacctgctaggggagctgctgttgactgcatcgcgatccaaaggaccggcgtcttttgtagatgcaggggctgagccaggccaagcgcgccgtggaggcccgcgtggacgcagacccgggtgccatacagatgcgctggaatccaggcactttcccgcgctccgcaaatctagatgtttcagcctggatcgagcgagggttagagggttagtcaggccagggtcaacttcagagtcatgggctccctaaatgcgacttctagggttgagttgctgtggacgagcgacccatgtcggaatcccgcgcccacgtggctgcccaaagttccgagtctccgggctgcaggttctagtcacggaaccgagttgggagagtcataggggctgggacttggaggatcggctgaggtccggtgctcttggctgtgttcgcggctcggagccgtcgcctgactgaggggcccgtcacagatgtgtgatgtataagctctgcacgcaacaggagctcaataaatgtgcgaaggggggtatacttatgttcgcactgtatgcaggcggcctagaaggaagtccctgattggcacagggatggaggatggggcaagagccgcaacagcgccgcggagttccaacgctgccggttccctggggtacgagcacagcctcaagcagcctcaagccctaggaagcccccagttcaaagcacagggcgcattggagcctgggcacgatacagttcacaccacggctgcgatggtaagccacgcccaagtcccaagggcctaggggacccccgccctccacagccggaggagaaacctgggcgcagaaagcagggggaatatctggttgtaggtgagtaagcggggtcaggagttcccgttagagtctctgcgtttcgggagaagggtgatcattcccaggcttgtccgacgtctctctcagggtgcgctccggaagagcgagccctttaaggctatgccgagtgggcgcgtcccggcctctcccgggagaggagaggcggggcggacctgtgtcccgcccccggcccggcccgcccccagtgcccgccccgcccccggcactcggccggcggcgcctttgatgttccgacccgccagctcgcggagccgctctgccccgcgccctagcccgcgcctgcagcccgcccaggcggagtcagcccgcgctccgcccgccgcgatccgagctcggaggttcggactccgggctcgccgccccccgggccggctccgcgccccgcactcccggcgcccagcgccccgcgccccggcgggcggagcgcaccatgccgcagctggactccggcgggggcggcgcgggcggcggcgacgacctcggcgcgccggacgagctgctggccttccaggatgaaggcgaggagcaggacgacaagagccgcgacagcgccgccggtcccgagcgcgacctggccgagctcaagtcgtcgctcgtgaacgagtccgagggcgcggccggcggcgcagggatcccgggggtcccgggggccggcgccggggcccgcggcgaggccgaggtgagcccccgccggcgccggctcctcccccgcggtcgccgcgccgcgccgccccagttgcgcgcggccctcggggtctccagcgcgcagagcgtccctgccccggcgtcggccccgacccccgcggtcccaccgcccctcactcccctccggttctccctccaggctctcgggcgggaacacgctgcgcagagactcttcccggacaaacttccagagcccctggaggacggtgagtttctgcccggcccggcttcccttcgtcgcgctcaggccctggcctcggtgggacggggacgccaaggaccgcggggagccgggtgcctcccccaccgcagctcaggaggcggcagaacccaggggtggagagtggggggcgggcttcccgggcgccgccgggtcgagtca

SEQ ID NO: 9 - DNA, 2000 bp, Artificial Sequence
  promoter sequence
gaccaatatggtgaaaccccatctctactaaacacaaaaaattagctgggcgtggtggtgcatgcctgtacttgggaggctgaggcaggagaatcgcttgaacccgggaggtgggagttgcagtgagccaagactgtgccattgtattccagcctgggcaacaagagagaaactccatcaaataaataaataaataaataggtcatggggattgatttcatcagattgcttatcagaatcaaaggaaatgattataggctttgaaaaatatagaccttatatttattattagtggattttgcaatattgaatcattcttgctaccctggaataaaccctacctggtgattatgtattttgtttaatatattatcggactctgcgtaacaatatttagaattttttcctcaacatatggaagtgagattcaaatctggtgttctttaattgtgtgcaatcttaatcactttaatatcagttttgaattcatatcataaaactaatgaagaaggctttatgttctatgtctgtatattttaagtaacattgaaattatctgctcttcagaagtttgttgtagtctctttgtgaaatcttctaggtatggcattctttttgctggggacaggaggagctcttgatgcttgagggagtcttggaatgatatgttcacgtgtacttctatcccagtcctagttgtcttggatgactgagggctctaggtaagaggaagaaggaaagaagtgtccaggggtagaggctacctttcccaaggaagatggggccgggatggattgatagtataaaggactgagagtaacaatgattctttgcttgaccaaactttgtcagcaaagtttgtcctgaaccttctcctaggcccatccatgcacttccttgtaaaatccattttttagccaagaacaaggataaatcagttttgcaagaacccctatccccctgtatctgatcacccttgatatcttatcaggtttctcatcctccaccatttctcagttcatgtctgatctaccctggcctgtctttagcaagaatcctattaggtcagtttagccagaatcttccttacacgtgatgttccctcttagcacttttccatccactgacccccactctgcttcttggtgataaattcccattgcccatgctgtattcagaattgagcccaatctctccccctctgcaaaatcccattgccttggtctctatacctattgtgatggtcctgaataaaggctgccttacctttaagaagtgtcattgcctaagttgtctaattatacgatagtttaatcccatctagaatagaggaggagaaggagggaagcaatgtgggtgtgtaggagggttgtgaagagaggcggcaggagaggcctaagctgagaggatcagccgcctcttcccaaagatttttcttctgactttgcattccaacctatcctaggggtctctgtcctattttcacagctggaaagaagaaagaaatttcccatagtcggttgaacttcttggcagccttggaggatggaggagatagatcctcagagccacctcacgggagggagttttgacaaacagaagtcagcggtcccactcccaagttctcctcatttgtatttgccagtgtcagccaagtcccatggcttgttaataagcctcagatcacgggcagtcctttcgcgtggagtctgattttctgaggcagctctctccacaagtggctttggaactccatcactcaggccgccccactgtgaggaagcttggctaaggctgtgagacagctgggggtgatgggacacagggcgggcccttcctggtgcctggggcggccaggtccccttgtcccccctgtgtcagggctcccgtgcttcctcgcccttctctcctaacatctaaggcaggaggcagggccttctcgccccccgccataatataacctgccaccaggaggcgctgttctacaggatggagagtgg

SEQ ID NO: 10 - DNA, 2000 bp, Artificial Sequence
  promoter sequence
tggtcactatttactgtgtgagttccactggagacaagcacacttgctccaatcccagagtctacacctgaccgggaatttttcattttggattctgattgagggaaaatactgatatccaatgggctataagggggacctttcagtttctgttaacagcatctgagttctatgcacgtcctggaaaggcacctgcccactggctaattcacatgttgtaatcccaacactgtaaatatctgacttcacattatacccatgtaaatcctgtctcagtagttctggactcagccatggctgcactaatgtgctgaactgtgggaaatcatacacagccctatgcctctgtccatgcttaaccaaactatgcagatgggacaggccagagagggtcactaggccatcaccagaaatgaggatatggctggctttaattctcctgtgaatacagccatattggtagagatagttcaatcctctcactgctccaaagagaatgtttcttattaaagttttactcattccttcaggaaaataggtcctcaagagttgtcttgctgaaccataggccataaatggagaaataacccaaagccagctgccaacagtgaaaactgtccaataagttgtaatattgggatgccggaaaaagtgggatagaatcatggctttctgtaaaactttcaggcgttctttattgccgttttccagatttgtaatttttgtagttaccagcgttcccgtaggagtatgccgtgctagatggacagaagtcaacttgtcaaatcctcttcctatttctacttggagctcgtagtgagaaacgttggcggaacatactacttcactggctctagtggatggaggcgaccaggaaagggttggctcttcgaccaagtattgatggatactggtttcagacttgtttttcatgtttgagtgattcaacttgtgttcttgaactaaagctcacagaaagtgacaaaaatccaaatattccaaagaaggcttttgttttatcttcctttgaagatgcagatatatcctacttagcggcgacgacggccagcgactccctacaggctgccagtggtgcggacctgggcgggtgccggagcccggatcacagaggggcaaggcgagcgctctaggcccactcctggcgtggggcgctggagggtggcaaggccgcgacaggggctgtgggcttggaactctaaacatagtattcttacgtagccaactggaatatctggtcaaccgtgagagctttgggaacctgcctctcacctcaagaaacctccagagcagattggaaccatccactgggtaaattctggaaaatctgtctctgcctgagactggggtaggaagatgtggggtagagaggaaggtggggaagggatgtaggatctattccttgggtgcatctttacttgttaggtcatgcatattttcaaacttggaagaattatacgaaaagcaactagagaaactatgtgttcatgggcagattatttttccaggcgtcaccttcctttttttcccttggttcctaatgatttagactctgcctttgagcctcagtgctgttaacttttgaatgagtaacttgaggccttcctgaaagactataataaaaaacccatattatacaatcactaagaacccccagctctacgcttggaaatggcaaggtatatgcctgtctctcttcttccccaaatcacccctcagcctcccactccaccctgggaagacaatcaggtcctgtgccttgttttctaggcaatatttgggtcatttaataaggctcttttgcatccatcactataacctgaagcgaaaaatgtagctttggaaatggtgtttatagcaggcccatgggcaaaacgtttcaaccgggcaaaactgaagcaatcaccagattatggagccgtatttctgtttctttaaagacaaacatttttgtgtgtgtggctccacccagcctgagcttc

SEQ ID NO: 11 - DNA, 776 bp, Artificial Sequence
  promoter sequence
gatctttctctcagctttccataatctctgagcgaagtacgtttggagaactggggttacaggggaaaaagccaggtgttaatgatgaaaaaacattcaacttttctaccgctactaataacatttaattcaagtactgagaacatttacctcaaatcttcaagaataagacaatattatcccctttctcttttattgtcggactagagaatgtgagagaggttacattccatgggctttgggaatttaatatggttcaaggataaacacacccaggtttttcactccagagaagagcttcaaatataatccagttttcaggtcatcagctcagctcttgtatccctaacaatgcggttgacataccgtcttctcacatagtctaaactcctaaactcactaagccatactttaaagtacatataaaggactagaagcaccaagctaccagtgagacgaagaggagagtttccacagaagctggcttcaaataagacaatgagttcatctttaaatacttgccatttgaggtgcagatggatatagttggcaggctcctatctaacgcatgttatgcacaagctaccgtgaattgataatatcaaaacaaatatccagcgagcctctgcaagtgtgcatctctatttcacaccaattatagttgacttaattcctccctcattcatctcccagagatgcagcctcctcttaaagaagttgcggctggtggcccattcagtgatgtcactgacagatgcattctctgggaaatgtca

SEQ ID NO: 12 - DNA, 2000 bp, Artificial Sequence
  promoter sequence
tagctccaaataacgtgagattaggtctcacattagggagatttctcatccctacttttgccttagatatcttgcaactcaggtaaaggataatgtggaggatcctacgaatagtaatcaggacccgctagcccagggatagatacgtcgtccaagccgcccaagctgcacggcctctttctttccttgagctcgcgtacccctcacgggatcttcacgttgacagtcatgaggtattcacagagctatactacttgaacaatcaatgttctagacttccttgggtcaaaataacactaaagcaggggaggaaggcatggacaatgtagtgatcaaaacagccgtgccaaacactaactatactgatgacacaaaacgcaggaagcgcagggcagaacgcgctcagtaccagctttctggcgatctgctcctggccactctgtcccaggacctacaaggccaccgccccagcccactagtctcaacttgccccctcccctgtggctgtgccttgaccctgaaaatcttcgcgcactcctgggtttcaccaccacacacacgcgagcgcgcgagcgcgcgaacacacacatcaaaaaagaaaaaaaaaaccgtgataaatgccctggagagtctaagtgcattaaggacctcgttctcggagaccccagacatgccgatgttggtgctgctgtggttggaaaactgggttggaaagcttcgcactgttctacacttgcgtgtgcgcactcagcaatcctttggccatctcatctgttgtgggcgaagagtttcccgtgtgatcgcgttcggttggggaagcagagtcccgacatctcagccggaaaatgcgctcccggagcgattactggcggcgtctgtaattgcttattaacagcgaatattcaggcttctccttatccgcaacgaaacgtgccccccgcttccgtaataatgaaacgataaaatatgacggccccgctcttgaatctatctgaggaaacgcagcgaagaaacaagcagctggagtttaattcaatatcaaactgattatttcactaattattctaccttctgtatttgccgcagagcagaggcgcagggaatcctaactgtggggctgttagggaagctttgtttcttggtgtcaggcctaactgaagatggtggtgaagggacggaatccgggatcaggaaaactgagcagaaaggattttaaattgtggggaataaaagtgactgtctccccgaaagcagttgtgttggcgtgagtatgaagttggtgttagctcgcttagtttcactcttgtcctgtccccagtaccctgtccaagcttctgatttagaagctgagagcttaggtcctccaggatggcaatgtgctctggccgtccccggaatcccaaaagcttagcgcgagtttcctctctacaaacctttcccactgggtaaaaggatgggaacaaacaaaaagtggttctgtgcgttgaaggccagggagtttgccttcgacaagaggaaggtgtgctttgaagttacagcttccgacgaggaaagagacttccttgggcctgtctccaacttgccccagtttccccagcctccgggacgggcgcttccctgcaagctatcagcttgaagagtctttctctctctggcctctaaaatctgctccagccatctcattcctccagcggagggctgagatgactaatgcgccaagggtggacaccgctatggagaccccaaactttgtccccctaaattccggcgaaattacagacaccaggattgattatcacagcgggacgcagtctcaaaaaaaaaaaaaaaaaaaaaaaaaaaaaaaggaaaagaaagtcacaggcgacttgatccaattagtaaatgcctaattgaattagtgtcacctccaccagccaataggagggtctccgggctgtgaggccgggaagcccctccctggccgcaaatatataaagcagctagtgacagcccataag

SEQ ID NO: 13 - DNA, 2000 bp, Artificial Sequence
  promoter sequence
ataatttctagccaaataaatgaggataagtgtgtgagtgtggtggggtagttaagagtttggaatccagagtttcactgtctggatttgaacaggctccgttgctcatcagctctgtgactccgggcaaatgactgaatctctctaactctcagttttctcatctatgaaatgagaataagtaacagactctccctcgctgagttgtttgagaacgggatcagacaatgcacgtggagccccgtgtctggcacacactatgtcctcattgcatatttattattaggtttctgctttgtatttttctctgtgcaacagccctgagctctagaacttatgacagcttttctcaagtgaagttaaagttccccatactcagttccagagctaggcctcacctaggttggaagaagtcaattgttctccactgcctgcctgtgtgctgggaaaggactctagcacagtgccggtggggctggaagcctttcctcatgatgctacggggagtacagtgaaaagggaggagattggggtagcaaggtcccaggggtcctggcagcccaggctctacttctcaggctagctcagtccctcaaacagcttccttcacacccactgctgagacccaggagccctacagaaggtagctaggggaagcttcagcagctagggggatccctgcaggatcagcctcctccttaccttgcagaccccttggctgtggtctggcattcctagtggctggcctaaagccccaaacctggaatctgtttcagagcctggaccttttggactcacaccttggtttccctggccagctgatttcagcttctcatctaaatgtcatttctgcaggctaagggaagtccagcctggatctagcatcttggaagcgcgttcagacttctggatggagggatcagcaggccttggtattggtggttctacctgcttcggttaagagtgaggactctggagccagactctctaaatcatagcactgacacttactatatgacttgggccaggttcgttacctgtctgtgcctcagttttcctcatctgtaaaatgggagattagtggtcaccattgggatgattaagaagattaaatgagttaatgtatgtataaactatagttataatgtaaatatatatacaaacagtaaccatttacatgcatataaaacataatataaatgtaatattagctatattcatatataaaaataggtatattttatatataaaatatattacatttaattttttacattttgcattaagatatattatttcatataaaacatttaagtacatatacatataattatctatgtaattattattgttattgttgttattgtctactttacctccctgcagggatggggagaccacggcaggcttttaaacattcttggtctcactgagccgtaggtgaggaatccctggttcctacagactctgctgtaatatgaggagcagggtttaagttagtttaaaactgaccccatcatttgaaacattgcaatctacaatgaatgccacataaatatcatttctcagattcctatgatgctcttctttcagatcttttcacttcaatttctataataattttgtttgtttcttgtcctatttcaaaggctttcttatctctggagcacctagcataagatagaaatgtgtcaaaatatatgttttattcatcatgtgagtatttttaggtcctgttaacccccataactattgattcagagaagtagggtggttctgaaaaatacaggcataatctctttaacttgttttataggaaccagaataagggtaatgttttcctctgtcttcaaaatcatcaataatccatgcattgtttaactcatgtcataagcaataatgcctttcatatagccattggcatcaaagaagaaacacccccttgatttgatggtaagcgtgacactacataaactcccagaaaacccacttcctttcca

SEQ ID NO: 14 - DNA, 2000 bp, Artificial Sequence
  promoter sequence
aaaaatacaaaagttagccaagcgtgatggtgcatgcctgtaatcccaactactcaggaggccgaggcaggagaattgcttgaacctgggaggtagaggttgcaggaaaaaaaaaaaaggtaatatttcagctgagccttgaaggttgagttagaattcagcagttggacaaaagagagcatgccgactgggttcggtggtgcatgcctataatcctagcatttggggaggccaaggcaggaggattgcttgagcccaggagttcgagatcagactgggcaacatagtgagacctcatctctacaaaaaaataatcagctgggcatgatggtgtgcacctttagtctcagctactggggaggttgaggtgggaagattgcttgagcccaggaggtctaaggggattgcgccactgcacttcagcctgggagaggaagggagaccctgtctcaagagagagagagaaagagagagagagagagaaagagagagagagagagaaagagagagagagagagagagagagagcgagagagcgcacgcatttcaaagcagggagaaccacaagtacaaaggcattaagtaagaaaatagcatggggtgtttgggaaaggcaagaatagttcagaacacaggctgtgtgtgtgggggtgtgtgggggtgcctgtgtgtgggtgtttgtgtgtgtgtgagtgtgggtgtgtgggggtatctatggaggggatgcctgtgtgtgtgtgtaaggtgagtgtgtggatgtgtgcatgggtgtgggtgagtgtgtggaggtgcctgtgtgtgtgtatgaatgtgtgttggggaggggagaatgtgtgtgtgtggggtgagtgcgtgtgtgtgggggtgaatgcgtgtgtgtgtgtatgtgggtgtgactgtgtgggtgtgtggacgagtgtgtgtgttcctgggtgtgggagcctgtgtgtgtagggggagtatgtatagggtgtgtgcatgaatgtgtgtgtgggtacgtgtgtgagagtgggtgcctgtgtgtggggggtgagtgtgtgtgtgggggggcacttgtggagggtgagtgtatgtgtttactgagtgtgagtgtgggtgcctgtgtgtgggagggtgagtctgtgtgtgagtgtgtgggggagtacctgtgaggggtgagtgtgtgtgtttatgtgaaagtgtgtgtgtgtggatgcctctgtggaggtgggatagggggtgcctctgtgtgtgtgtgtgagagtgtgtgtgtgtagggtgtgtatatgtatagggtgtgtgtgagtgtgtgtgtgtgagagagtgtgtgtgtggcagaatagactgcggaggtggatttcatcttgatatgaaaggtctggaatgcatggtacattaaactttgaggacagcgctttccaagcactctgaggagcagccctagagaaggaggagctgcagggactccgggggcttcaaagtgagggccccactctgcttcaggcaaaacaggcacacatttatcactttatctatggagttctgcttgatttcatcagacaaaaaatttccactgctaaaacaggcaaataaacaaaaaaaaagttatggccaacagagtcactggagggttttctgctggggagaagcaagcccgtgtttgaaggaaccctgtgagatgactgtgggctgtgtgaggggaacagcgggggcttgatggtggacttcgggagcagaagcctctttctcagcctcctcagctagacaggggaattataataggaggtgtggcgtgcacacctctccagtaggggagggtctgataagtcaggtctctcccaggcttgggaaagtgtgtgtcatctctaggaggtggtcctcccaacacagggtactggcagagggagagggagggggcagaggcaggaagtgggtaactagactaacaaaggtgcctgtggcggtttgcccatcccaggtgggagggtggggctagggctcaggggccgtgtgtgaattta

SEQ ID NO: 15 - DNA, 2000 bp, Artificial Sequence
  promoter sequence
ttggctcctatgaagtgaactttttaaaaccaaaacaaacaaagggactatttgtttttgtttatgttttccttaaagtgagaaaaaaatattgagcctcctaagtttgcatttaaaagttggcaaagatgagtctcagttagtgggtttgatggctctatgagaaagagaataaaattacttacgatgacactccatgtagactgagcaaacgaaagactcctaagtattgcaacagctactgatgatattttgttttcccggatgagaacactttgtgaagtctataactcttttcatctgaaccaagcaggtgtaaaatgtactttcctcagaggagtttgtagtctagtttgagaagatagcactgtattatctaggtttgctggtagtgacagaaacctcaaaataactgagacttagaaaatagatattaaaaaatgtaatccagggctggcatggaagatccatggtcaccagggtcccaggctctttctatcttatactccattgtcctcaacattcaatttccacctcatgatactagatagctgcttcagctccaactgttacctcaatattctagtcacaaggcaggaggaaaagggaaagaaaaggggatgacagatcccactaaggaaatttcctagaagttgtgcacacgacttctgctacattcagttgaccagaagacagtcatacaactacacctagctacaagggaggctaaaatttgtagtctttattcttgctggccagatgcccagataactgtcagggattctgttactctagtaagaaggagagagtagctatttgggaatagtagcaatccctacctcaagcccatactcagaaaaaaaaaatactatcttgtttgggcagtgtatttccacacatatgtcttaagaatctcagaatgggggaggtaatttaaaatataccagtaccgtaccctcctgggtgtcttgattatgctggggcatccagtatatgaaataaaattaggctgttgtctggtaatttacatttaaaaataaacattggagaatttcactcctatacagcaataattattatttcatttattatttatccctacccactacatgcaatatctgcttacactaaaatctcttgattattaacatagtagaggtaactgtcacacattatacttcaactatgtactaactctaaggtgacatgtatgactagcacctaccattcataagatgtatgggattcagtcctccaaaggaccctcacaaggaagctaaggatcagagaagtcatggcttgctcaaagagaaacagcatagtaagtgacagagctgggaatccaacacaagtttgtctgttttcaaagcccacatatactttgtttattatgccattctgaaggattcacttatttttcactcttccacaaaacgtcaggacagaaaggcagtcatctatatgtagcattgagaaatttctatgggctttaatttaaatcttaaaaactaataaacttcagcaacatatttcttcactttaatgtgtgagggttttgtgctcattacctcttttaattgtccaacccaatttggtgatgatccattcaacaaatttttcttgaacatctgttttatgtgtcaggtactgtgatggttgctgagtatatcatggtaagcaaaaccacacattgtccttaccccttgtgctacatgatttaactgctggatggattaaaccaggaactataaagattaaactgtaatccttaccacaattttgagtttacagtttttcaaatatcatttcaagccattagcaaaggctgtatgtattttttacatatgcctcctcgttttgtgaattttgaaaggatgtggtttcggcctttgacatcagaggagaagctcagctatgttggctgaacgttgatagaaagataacgttgaaggcaagttgcccttgagcagctctctgaagatcaactgcctccacattgc

SEQ ID NO: 16 - DNA, 2000 bp, Artificial Sequence
  promoter sequence
acacccccaacttcttctcacatccctgcatcgtgccatgcagcatcaactggaaacctcagcatcagcaaacgacgacagagcgttcatccgtaaggtgaaccagaaaagccagttcaatgacttgtttaaccatggtccatctcagaaccaagagttgggcctcttatttaccagaaaaattgtgggggctttgtgatatggctttaaaaaaatcttgtaattgccaggcgtggtggctcacacctgtaatcccagcactttgggaggccgaggtgggtgaatcgcctaaggtcaggagttcgagaccagcctgaccaacatggtgaaactccgtctctactaaaaatacaaaaactagctggatgtggtgacgcgtgcctgtaatcctagctactcaggaggctgacgcaggagaatcacttgaacctgggaggcagaggttgcagtgagccaagattgtgccattgcgctccaaaaaaaaaaaaaaaaagacattaacataaatttaaatattttataatgacaatccacattaactacttaaagcataagctattttccaggagaggcagcaagtgcattctactcccatgcccaagaagaaaggagcgtgactttggtgggagtactaggagtttctactggagcacttgcccgcagagtgagaaacgttcctagagaggaagttatacctgctgtggaatttaagagaatcttgtcatattttgacaagttttttgagatggaagtctcactctgtcgcccaggctggagtgcagtggcgcaatctcagctcactgcagcctgcacctcctcggttccagctattctcttgtctcagcctcctgagtaactgggattacaggcgcccgccactacgcctggctaatttttgtatttttagtagaaatggggttttaccatgttggccagactggtctcaaactcccgacctcaggtgatctgcctgcctcagcctcccaaagtgctggaattacaggcgtgtgccactgcgcctggctaatttttttttttttttttttttttttttttttttagtagagacggtggtttcaccatgtcatccaggctggtctcaaactcctgacctcaggtgatccacccaccttggtctaccaaagtgctcggattacaggcatgagccaccaggcccagtcaacgtgatgtgttttggaaccctgaattccttggcttgcccggagggttttctttttgttaatatctttgcttgctttctagtatttaaaaaattgtgttttgctctaactatgcaatggctttaagtcttagacaaatttccagggagcaaaacacactcaaccatttcataataatcagaagagagctctgatcaataaataagcaagactgaattttacaaaataatccaaagtttaaaaccaaagcccactttttgcatgatcctttaagagaaagaaatctggaagcaaaacaccttataaaatgacaatgcactttcaggagcccagggcactgtggtgaaatgatgatggctagtacaggttataagccttggggaattatttatgaattctcaggatccttcagttcgccgcatccttctccattatttgaatattggaggctgcctgaccagaatcttgtcaggactttgctccttcatcccaggtggtcccggctgactcctgaggacgttacagccctgaggggaggactcagcttatgaagtgctgggtgagaccactgccaagaagtgcttgctcaccctaccttcaacggcaggggaatctccctctccttttatgggcgtagctgaagaaaggattcataaatgaagttcaatccttctcatcaaccccagcccacacctccagcaattgaacttgaaaaaaaaaacctggtttgaaaaattaccgcaaactatattgtcatcaaaaaaaaaaaaaaaaaaaaacacttcctatatttgagatgagagaagagagtgctaggca

SEQ ID NO: 17 - protein, 4 aa, Artificial Sequence
  oligopeptide for Modified Poly(beta-amino ester)s
CRRR

SEQ ID NO: 18 - protein, 4 aa, Artificial Sequence
  oligopeptide for Modified Poly(beta-amino ester)s
CHHH

SEQ ID NO: 19 - DNA, 1844 bp, Artificial Sequence
  promoter sequence
ccttgctgctaagagcatccgcttgcaccttctgctcatccccagacaagctttgtcctgtgaccataatgaactcttcatgccgtttccaactttagcccatgttattcttcttgtctgaatatccacccttttctctgttctcaataataagttcaggcttttcgtcttctgagaagccctttctgacttccacaggctgaaccactggcttctgctcctctacataatacttcaattccagcattgatctcactctatcatgatcatgggtttagctgtctgtccctgccactgctgtgtgttcctcttgagggcaggaacatttgtttttcactttttaaaaaacctctgttgcccagtctggcattaggaagtgcccattaggttgttattgcttgttggcgcttgagctggggcttgaaggtttctataatgtgtagcagtgtatagaaaacaggcaggtcagaaaaggcttctgtgcatcacaccaacatggcacatgtatacatatgtaacaaatctgcatgttgtgcacatgtaccctaaaacttaaagtataataataataaaattttaaaaaaaaaaagaagaggcttcctggaggagatgacagctgagctaagtcctggaggatgagaaggagtataaaataagataataggagaaaaaaggcagtaggaacagcatgggtaaaggtgatgaggcctgaaagaggcacgtggaaggaaagacaaatgcaggaagggggaatgggagggaatgctggggtacaggccaaagagggaggcatttggtgagtattctgcagagtctcctctgctgtgctgaggtgtggacaatgggaaaccatggacggactggagtaggcaaatgtcatattccctgttacaactgtctgtttgcatgtcagccttctagaagccccttaaggtatcaactatgtttttgttttgtcatcattcaatcctaagtgcacagaattccgggcatattacaggttccccatgaatgtttctttctttattaaaatgtatgaaaactctccagatttaaggaaggtcctcaatgtttcaaattctttttgttagatcattggtcctgtctacagctgtcacaaatttaaggactctggttatatttaatcttcacttttgaattttctgcttgaaaaatttgtattagaaaaaaaagtctatccttttatggacggctctaatctcttgaatcatttgggttggcttttctttggaccttcttcaactctgttttgtctctgttgagttaaggcttttaagaacacctgaattctttccttctgcaaaaccagaggcagcttcttttccgcctattttcagtttatttcttgtgattttagtttttttctcttaaccaaatgctaaatggatttaggagaaataaacttatttgtaaagctgtcaagggaccattagaaggatggtgcttcacagatagaatacagtttttattaatgatgcctagacaaatcctgccattagcccaagggctcagaaagttagcagcctagtagttttggagttgtcaatgaaatgaattggactggatggttaaggatgcccagaagattgaataaaattgggatttaggaggacccttgtactccaggaaattctccaagtctccacttagttatccagatcctcaaagtgaacatgaagcttcagtttcaaattgaatacattttccatccatggattggcttgttttgttcagttgagtgcttgaggttgtcttttcgacgtaacagctaaacccacggcttcctttctcgtaaaaccaaaacaaaaaggctttctattcaa

SEQ ID NO: 20 - DNA, 1237 bp, Artificial Sequence
  promoter sequence
agatgacagctgagctaagtcctggaggatgagaaggagtataaaataagataataggagaaaaaaggcagtaggaacagcatgggtaaaggtgatgaggcctgaaagaggcacgtggaaggaaagacaaatgcaggaagggggaatgggagggaatgctggggtacaggccaaagagggaggcatttggtgagtattctgcagagtctcctctgctgtgctgaggtgtggacaatgggaaaccatggacggactggagtaggcaaatgtcatattccctgttacaactgtctgtttgcatgtcagccttctagaagccccttaaggtatcaactatgtttttgttttgtcatcattcaatcctaagtgcacagaattccgggcatattacaggttccccatgaatgtttctttctttattaaaatgtatgaaaactctccagatttaaggaaggtcctcaatgtttcaaattctttttgttagatcattggtcctgtctacagctgtcacaaatttaaggactctggttatatttaatcttcacttttgaattttctgcttgaaaaatttgtattagaaaaaaaagtctatccttttatggacggctctaatctcttgaatcatttgggttggcttttctttggaccttcttcaactctgttttgtctctgttgagttaaggcttttaagaacacctgaattctttccttctgcaaaaccagaggcagcttcttttccgcctattttcagtttatttcttgtgattttagtttttttctcttaaccaaatgctaaatggatttaggagaaataaacttatttgtaaagctgtcaagggaccattagaaggatggtgcttcacagatagaatacagtttttattaatgatgcctagacaaatcctgccattagcccaagggctcagaaagttagcagcctagtagttttggagttgtcaatgaaatgaattggactggatggttaaggatgcccagaagattgaataaaattgggatttaggaggacccttgtactccaggaaattctccaagtctccacttagttatccagatcctcaaagtgaacatgaagcttcagtttcaaattgaatacattttccatccatggattggcttgttttgttcagttgagtgcttgaggttgtcttttcgacgtaacagctaaacccacggcttcctttctcgtaaaaccaaaacaaaaaggctttctattcaa

SEQ ID NO: 21 - DNA, 1048 bp, Artificial Sequence
  promoter sequence
gtgagtattctgcagagtctcctctgctgtgctgaggtgtggacaatgggaaaccatggacggactggagtaggcaaatgtcatattccctgttacaactgtctgtttgcatgtcagccttctagaagccccttaaggtatcaactatgtttttgttttgtcatcattcaatcctaagtgcacagaattccgggcatattacaggttccccatgaatgtttctttctttattaaaatgtatgaaaactctccagatttaaggaaggtcctcaatgtttcaaattctttttgttagatcattggtcctgtctacagctgtcacaaatttaaggactctggttatatttaatcttcacttttgaattttctgcttgaaaaatttgtattagaaaaaaaagtctatccttttatggacggctctaatctcttgaatcatttgggttggcttttctttggaccttcttcaactctgttttgtctctgttgagttaaggcttttaagaacacctgaattctttccttctgcaaaaccagaggcagcttcttttccgcctattttcagtttatttcttgtgattttagtttttttctcttaaccaaatgctaaatggatttaggagaaataaacttatttgtaaagctgtcaagggaccattagaaggatggtgcttcacagatagaatacagtttttattaatgatgcctagacaaatcctgccattagcccaagggctcagaaagttagcagcctagtagttttggagttgtcaatgaaatgaattggactggatggttaaggatgcccagaagattgaataaaattgggatttaggaggacccttgtactccaggaaattctccaagtctccacttagttatccagatcctcaaagtgaacatgaagcttcagtttcaaattgaatacattttccatccatggattggcttgttttgttcagttgagtgcttgaggttgtcttttcgacgtaacagctaaacccacggcttcctttctcgtaaaaccaaaacaaaaaggctttctattcaa

SEQ ID NO: 22 - DNA, 745 bp, Artificial Sequence
  promoter sequence
tcctgtctacagctgtcacaaatttaaggactctggttatatttaatcttcacttttgaattttctgcttgaaaaatttgtattagaaaaaaaagtctatccttttatggacggctctaatctcttgaatcatttgggttggcttttctttggaccttcttcaactctgttttgtctctgttgagttaaggcttttaagaacacctgaattctttccttctgcaaaaccagaggcagcttcttttccgcctattttcagtttatttcttgtgattttagtttttttctcttaaccaaatgctaaatggatttaggagaaataaacttatttgtaaagctgtcaagggaccattagaaggatggtgcttcacagatagaatacagtttttattaatgatgcctagacaaatcctgccattagcccaagggctcagaaagttagcagcctagtagttttggagttgtcaatgaaatgaattggactggatggttaaggatgcccagaagattgaataaaattgggatttaggaggacccttgtactccaggaaattctccaagtctccacttagttatccagatcctcaaagtgaacatgaagcttcagtttcaaattgaatacattttccatccatggattggcttgttttgttcagttgagtgcttgaggttgtcttttcgacgtaacagctaaacccacggcttcctttctcgtaaaaccaaaacaaaaaggctttctattcaa

SEQ ID NO: 23 - DNA, 344 bp, Artificial Sequence
  promoter sequence
tagacaaatcctgccattagcccaagggctcagaaagttagcagcctagtagttttggagttgtcaatgaaatgaattggactggatggttaaggatgcccagaagattgaataaaattgggatttaggaggacccttgtactccaggaaattctccaagtctccacttagttatccagatcctcaaagtgaacatgaagcttcagtttcaaattgaatacattttccatccatggattggcttgttttgttcagttgagtgcttgaggttgtcttttcgacgtaacagctaaacccacggcttcctttctcgtaaaaccaaaacaaaaaggctttctattcaa

SEQ ID NO: 24 - DNA, 1703 bp, Artificial Sequence
  promoter sequence
gtatttttctctgtgcaacagccctgagctctagaacttatgacagcttttctcaagtgaagttaaagttccccatactcagttccagagctaggcctcacctaggttggaagaagtcaattgttctccactgcctgcctgtgtgctgggaaaggactctagcacagtgccggtggggctggaagcctttcctcatgatgctacggggagtacagtgaaaagggaggagattggggtagcaaggtcccaggggtcctggcagcccaggctctacttctcaggctagctcagtccctcaaacagcttccttcacacccactgctgagacccaggagccctacagaaggtagctaggggaagcttcagcagctagggggatccctgcaggatcagcctcctccttaccttgcagaccccttggctgtggtctggcattcctagtggctggcctaaagccccaaacctggaatctgtttcagagcctggaccttttggactcacaccttggtttccctggccagctgatttcagcttctcatctaaatgtcatttctgcaggctaagggaagtccagcctggatctagcatcttggaagcgcgttcagacttctggatggagggatcagcaggccttggtattggtggttctacctgcttcggttaagagtgaggactctggagccagactctctaaatcatagcactgacacttactatatgacttgggccaggttcgttacctgtctgtgcctcagttttcctcatctgtaaaatgggagattagtggtcaccattgggatgattaagaagattaaatgagttaatgtatgtataaactatagttataatgtaaatatatatacaaacagtaaccatttacatgcatataaaacataatataaatgtaatattagctatattcatatataaaaataggtatattttatatataaaatatattacatttaattttttacattttgcattaagatatattatttcatataaaacatttaagtacatatacatataattatctatgtaattattattgttattgttgttattgtctactttacctccctgcagggatggggagaccacggcaggcttttaaacattcttggtctcactgagccgtaggtgaggaatccctggttcctacagactctgctgtaatatgaggagcagggtttaagttagtttaaaactgaccccatcatttgaaacattgcaatctacaatgaatgccacataaatatcatttctcagattcctatgatgctcttctttcagatcttttcacttcaatttctataataattttgtttgtttcttgtcctatttcaaaggctttcttatctctggagcacctagcataagatagaaatgtgtcaaaatatatgttttattcatcatgtgagtatttttaggtcctgttaacccccataactattgattcagagaagtagggtggttctgaaaaatacaggcataatctctttaacttgttttataggaaccagaataagggtaatgttttcctctgtcttcaaaatcatcaataatccatgcattgtttaactcatgtcataagcaataatgcctttcatatagccattggcatcaaagaagaaacacccccttgatttgatggtaagcgtgacactacataaactcccagaaaacccacttcctttcca

SEQ ID NO: 25 - DNA, 1443 bp, Artificial Sequence
  promoter sequence
agcccaggctctacttctcaggctagctcagtccctcaaacagcttccttcacacccactgctgagacccaggagccctacagaaggtagctaggggaagcttcagcagctagggggatccctgcaggatcagcctcctccttaccttgcagaccccttggctgtggtctggcattcctagtggctggcctaaagccccaaacctggaatctgtttcagagcctggaccttttggactcacaccttggtttccctggccagctgatttcagcttctcatctaaatgtcatttctgcaggctaagggaagtccagcctggatctagcatcttggaagcgcgttcagacttctggatggagggatcagcaggccttggtattggtggttctacctgcttcggttaagagtgaggactctggagccagactctctaaatcatagcactgacacttactatatgacttgggccaggttcgttacctgtctgtgcctcagttttcctcatctgtaaaatgggagattagtggtcaccattgggatgattaagaagattaaatgagttaatgtatgtataaactatagttataatgtaaatatatatacaaacagtaaccatttacatgcatataaaacataatataaatgtaatattagctatattcatatataaaaataggtatattttatatataaaatatattacatttaattttttacattttgcattaagatatattatttcatataaaacatttaagtacatatacatataattatctatgtaattattattgttattgttgttattgtctactttacctccctgcagggatggggagaccacggcaggcttttaaacattcttggtctcactgagccgtaggtgaggaatccctggttcctacagactctgctgtaatatgaggagcagggtttaagttagtttaaaactgaccccatcatttgaaacattgcaatctacaatgaatgccacataaatatcatttctcagattcctatgatgctcttctttcagatcttttcacttcaatttctataataattttgtttgtttcttgtcctatttcaaaggctttcttatctctggagcacctagcataagatagaaatgtgtcaaaatatatgttttattcatcatgtgagtatttttaggtcctgttaacccccataactattgattcagagaagtagggtggttctgaaaaatacaggcataatctctttaacttgttttataggaaccagaataagggtaatgttttcctctgtcttcaaaatcatcaataatccatgcattgtttaactcatgtcataagcaataatgcctttcatatagccattggcatcaaagaagaaacacccccttgatttgatggtaagcgtgacactacataaactcccagaaaacccacttcctttcca

SEQ ID NO: 26 - DNA, 1219 bp, Artificial Sequence
  promoter sequence
ggaccttttggactcacaccttggtttccctggccagctgatttcagcttctcatctaaatgtcatttctgcaggctaagggaagtccagcctggatctagcatcttggaagcgcgttcagacttctggatggagggatcagcaggccttggtattggtggttctacctgcttcggttaagagtgaggactctggagccagactctctaaatcatagcactgacacttactatatgacttgggccaggttcgttacctgtctgtgcctcagttttcctcatctgtaaaatgggagattagtggtcaccattgggatgattaagaagattaaatgagttaatgtatgtataaactatagttataatgtaaatatatatacaaacagtaaccatttacatgcatataaaacataatataaatgtaatattagctatattcatatataaaaataggtatattttatatataaaatatattacatttaattttttacattttgcattaagatatattatttcatataaaacatttaagtacatatacatataattatctatgtaattattattgttattgttgttattgtctactttacctccctgcagggatggggagaccacggcaggcttttaaacattcttggtctcactgagccgtaggtgaggaatccctggttcctacagactctgctgtaatatgaggagcagggtttaagttagtttaaaactgaccccatcatttgaaacattgcaatctacaatgaatgccacataaatatcatttctcagattcctatgatgctcttctttcagatcttttcacttcaatttctataataattttgtttgtttcttgtcctatttcaaaggctttcttatctctggagcacctagcataagatagaaatgtgtcaaaatatatgttttattcatcatgtgagtatttttaggtcctgttaacccccataactattgattcagagaagtagggtggttctgaaaaatacaggcataatctctttaacttgttttataggaaccagaataagggtaatgttttcctctgtcttcaaaatcatcaataatccatgcattgtttaactcatgtcataagcaataatgcctttcatatagccattggcatcaaagaagaaacacccccttgatttgatggtaagcgtgacactacataaactcccagaaaacccacttcctttcca

SEQ ID NO: 27 - DNA, 1000 bp, Artificial Sequence
  promoter sequence
ctgacacttactatatgacttgggccaggttcgttacctgtctgtgcctcagttttcctcatctgtaaaatgggagattagtggtcaccattgggatgattaagaagattaaatgagttaatgtatgtataaactatagttataatgtaaatatatatacaaacagtaaccatttacatgcatataaaacataatataaatgtaatattagctatattcatatataaaaataggtatattttatatataaaatatattacatttaattttttacattttgcattaagatatattatttcatataaaacatttaagtacatatacatataattatctatgtaattattattgttattgttgttattgtctactttacctccctgcagggatggggagaccacggcaggcttttaaacattcttggtctcactgagccgtaggtgaggaatccctggttcctacagactctgctgtaatatgaggagcagggtttaagttagtttaaaactgaccccatcatttgaaacattgcaatctacaatgaatgccacataaatatcatttctcagattcctatgatgctcttctttcagatcttttcacttcaatttctataataattttgtttgtttcttgtcctatttcaaaggctttcttatctctggagcacctagcataagatagaaatgtgtcaaaatatatgttttattcatcatgtgagtatttttaggtcctgttaacccccataactattgattcagagaagtagggtggttctgaaaaatacaggcataatctctttaacttgttttataggaaccagaataagggtaatgttttcctctgtcttcaaaatcatcaataatccatgcattgtttaactcatgtcataagcaataatgcctttcatatagccattggcatcaaagaagaaacacccccttgatttgatggtaagcgtgacactacataaactcccagaaaacccacttcctttcca

SEQ ID NO: 28 - DNA, 837 bp, Artificial Sequence
  promoter sequence
cagtaaccatttacatgcatataaaacataatataaatgtaatattagctatattcatatataaaaataggtatattttatatataaaatatattacatttaattttttacattttgcattaagatatattatttcatataaaacatttaagtacatatacatataattatctatgtaattattattgttattgttgttattgtctactttacctccctgcagggatggggagaccacggcaggcttttaaacattcttggtctcactgagccgtaggtgaggaatccctggttcctacagactctgctgtaatatgaggagcagggtttaagttagtttaaaactgaccccatcatttgaaacattgcaatctacaatgaatgccacataaatatcatttctcagattcctatgatgctcttctttcagatcttttcacttcaatttctataataattttgtttgtttcttgtcctatttcaaaggctttcttatctctggagcacctagcataagatagaaatgtgtcaaaatatatgttttattcatcatgtgagtatttttaggtcctgttaacccccataactattgattcagagaagtagggtggttctgaaaaatacaggcataatctctttaacttgttttataggaaccagaataagggtaatgttttcctctgtcttcaaaatcatcaataatccatgcattgtttaactcatgtcataagcaataatgcctttcatatagccattggcatcaaagaagaaacacccccttgatttgatggtaagcgtgacactacataaactcccagaaaacccacttcctttcca

SEQ ID NO: 29 - DNA, 550 bp, Artificial Sequence
  promoter sequence
cctggttcctacagactctgctgtaatatgaggagcagggtttaagttagtttaaaactgaccccatcatttgaaacattgcaatctacaatgaatgccacataaatatcatttctcagattcctatgatgctcttctttcagatcttttcacttcaatttctataataattttgtttgtttcttgtcctatttcaaaggctttcttatctctggagcacctagcataagatagaaatgtgtcaaaatatatgttttattcatcatgtgagtatttttaggtcctgttaacccccataactattgattcagagaagtagggtggttctgaaaaatacaggcataatctctttaacttgttttataggaaccagaataagggtaatgttttcctctgtcttcaaaatcatcaataatccatgcattgtttaactcatgtcataagcaataatgcctttcatatagccattggcatcaaagaagaaacacccccttgatttgatggtaagcgtgacactacataaactcccagaaaacccacttcctttcca

(19) United States
(12) Patent Application Publication
(10) Pub. No.: US 2023/0140346 A1
(43) Pub. Date: May 4, 2023

(54) Promoter Sequences for In Vitro and In Vivo Expression of Gene Therapy Products in CD3+ Cells

(71) Applicant: Ixaka France, Villejuif (FR)

(72) Inventors: Cécile BAUCHE, Paris (FR); Frédéric MOURLANE, Nice (FR); Renaud VAILLANT, Gentilly (FR); Rachel PACHERIE, Sceaux (FR)

(21) Appl. No.: 17/925,892

(22) PCT Filed: May 19, 2021
(86) PCT No.: PCT/IB2021/000343

§ 371 Date: Nov. 17, 2022

Related U.S. Application Data
(60) Provisional application No. 63/026,964, filed on May 19, 2020.

(51) Int. Cl. C12N 15/113; C12N 15/86; A61P 37/02

(52) U.S. Cl. CPC C12N 15/113; C12N 15/86; A61P 37/02; C12N 2830/008; C12N 2740/16045; C12N 2320/32

(57) ABSTRACT

Promoter sequences for use in expressing a transgene in CD3+ cells are provided. The promoter sequences can be inserted into a vector in a 5′ untranslated region proximal to a transgene. The promoters are selective for expression in CD3+ cells and contain binding sites for transcription factors found in CD3+ cells. The promoters can be integrated into vectors, including polymer-encapsulated lentiviral vector nanoparticles, used to transduce T-cells for genetic immunotherapy to treat cancer and infectious diseases. The T-cell selectivity of the promoters adds an improved safety factor to the use of viral vectors for immunotherapy in vitro and in vivo.

### BACKGROUND

There is great interest in developing the ability to modify the activity of specific classes of immune cells by targeted expression of exogenous genes introduced into these cells. Different methods are available for introducing DNA encoding desired transgenes into specific cell types, such as CD3+ T-cells, including plasmids and viral vectors, which are often packaged into delivery vehicles such as targeted nanoparticles. However, even if a transgene can be delivered successfully and specifically into the desired cells, there remains a need to present the transgene in a context suitable for sufficient expression to occur in the target cells. To that end, there is a need to develop promoter sequences that can drive required levels of expression of transgenes in specific populations of cells, such as CD3+ cells, so that immunotherapeutic and other treatment paradigms can be carried out successfully.

### SUMMARY

The present technology provides several promoter sequences suitable to drive expression of a transgene in CD3+ T-cells. The promoter sequences can be used in vectors, such as viral vectors, when placed 5′ to the open reading frame of a transgene intended for expression in CD3+ T-cells. Preferably, the expression supported by the promoters of the present technology is selective for CD3+ T-cells. For example, the promoters preferably support expression of the transgene at a level which is about 2-fold, 3-fold, 4-fold, 5-fold, 6-fold, 7-fold, 8-fold, 9-fold, 10-fold, 15-fold, 20-fold, 30-fold, or even 50-fold or 100-fold higher in CD3+ cells, such as CD3+ T-cells than in CD3− cells, such as CD19+, CD3− B-cells.

Promoters of the present technology include nucleic acid sequences, such as DNA or RNA oligonucleotide sequences comprising or consisting of the nucleotide sequence of any of SEQ ID NOS:2-16 or 19-29, or comprising or consisting of a sequence variant thereof having at least 80%, at least 85%, at least 90%, at least 95%, at least 97%, at least 98%, or at least 99% identity to any of SEQ ID NOS:2-16 or 19-29.

Other promoters of the present technology can comprise or consist of one or more fragments of any of SEQ ID NOS:2-16 or 19-29, or combinations of such fragments assembled in any order, optionally including linker sequences between the fragments. Such fragments can be any 10 or more, 15 or more, 20 or more, 25 or more, 30 or more, 40 or more, 50 or more, 100 or more, 150 or more, 200 or more, 250 or more, 300 or more, 350 or more, 400 or more, 500 or more, 600 or more, or 700 or more consecutive nucleotides of any of SEQ ID NOS:2-16 or 19-29. Fragments can be taken from the 5′ end or the 3′ end of any of SEQ ID NOS:2-16 or 19-29. Fragments can comprise or consist of the 100, 200, 300, 400, 500, 600, or 700 consecutive nucleotides selected starting at the 3′ end of any of SEQ ID NOS:1-16 and 19-29 and moving towards the 5′ end. Fragments also can comprise or consist of the 500, 1000, or 1500 consecutive nucleotides selected starting at the 3′ end of any of SEQ ID NOS:1-10, 12-16, and 19-29 and moving towards the 5′ end. Fragments also can comprise or consist of any binding site of a transcription factor, such as transcription factors NF-kappaB, AP-1, STAT, GATA-3, and NFAT, as identified in SEQ ID NOS:2-16 and 19-29, which can be combined with or inserted into any of SEQ ID NOS:2-16 or 19-29, or fragments or variants thereof as defined above. Preferably, promoters of the present technology contain at least 1, 2, 3, 4, 5, 6, 7, 8, 9, 10, 11, 12, 13, 14, 15, 16, 18, or 20 transcription factor binding sites selected from binding sites for one or more of NF-kappaB, AP-1, STAT, GATA-3, and NFAT, or variants thereof having at least 70%, 75%, 80%, 85%, 90%, 95%, 97%, 98%, or 99% identity to any of the transcription factor binding sites shown in any of SEQ ID NOS:2-16 and 19-29. The transcription factor binding sites can be positioned within the promoter sequence at any desired position in the sequence and in any order. Binding sites for other transcription factors also can be included in promoters of the present technology. Preferred promoter sequences are ICOS (SEQ ID NO:13) and CTLA4 (SEQ ID NO:7). Other preferred promoter sequences are the CD3+ specific promoters LAIR2 (SEQ ID NO:2), TNFS8 (SEQ ID NO:3), TCR (SEQ ID NO:11) and LTK (SEQ ID NO:14); each of these promoters contains fewer than 2 NFκB sites, fewer than 8 NFAT boxes, and fewer than 8 NFκB+AP1 sites.

The technology can be further summarized in the following list of features.

1. A promoter sequence for use in expression of a transgene under control of the promoter sequence in a CD3+ cell, the promoter sequence comprising nucleotides 1501-2000 of any of SEQ ID NOS:2-10 or 12-16, or a variant thereof having at least 90% identity to said sequence.
2. The promoter sequence of feature 1, wherein the promoter sequence comprises nucleotides 1001-2000 of any one of SEQ ID NOS:2-10 or 12-16 or a variant thereof having at least 90% identity to said sequence.
3. The promoter sequence of feature 2, wherein the promoter sequence comprises nucleotides 501-2000 of any one of SEQ ID NOS:2-10 or 12-16 or a variant thereof having at least 90% identity to said sequence.
4. The promoter sequence of feature 3, wherein the promoter sequence comprises the nucleotide sequence of any one of SEQ ID NOS:2-16 or a variant thereof having at least 90% identity to said sequence.
5. The promoter sequence of any of features 1-4, wherein the promoter sequence comprises a binding sequence for one or more transcription factors selected from the group consisting of NF-kappaB, AP-1, STAT, GATA-3, and NFAT.
6. The promoter sequence of any of features 1-5, wherein the promoter is capable of expressing the transgene at a higher level in CD3+ cells compared to CD3− cells.
7. The promoter sequence of feature 6, wherein the ratio of expression in CD3+ cells to CD3-cells is at least 2:1.
8. A promoter sequence for use in expression of a transgene under control of the promoter sequence in a CD3+ cell, the promoter sequence comprising SEQ ID NO: 2, SEQ ID NO:3, SEQ ID NO:7, SEQ ID NO:11, or SEQ ID NO:13.
9. A vector, plasmid, or nucleic acid molecule comprising the promoter sequence of any of features 1-8.
10. The vector of feature 9 which is a viral vector.
11. The viral vector of feature 10 which is a retrovirus, a lentivirus, an adenovirus, an adeno-associated virus, or a herpes simplex virus.
12. The viral vector of any of features 9-11 which is incorporated into a nanoparticle.
13. The viral vector of any of features 9-11 which is not incorporated into a nanoparticle.
14. The viral vector of any of features 9-11 whose envelope lacks a fusion protein.
15. The vector of any of features 9-14, wherein the vector comprises a transgene encoding a product selected from the group consisting of chimeric antigen receptors (CARs), checkpoint inhibitors, cytokines, chemokines, antibodies and antigen binding fragments and variants thereof, enzymes, structural proteins, and reporter genes.
16. The nucleic acid molecule of feature 9 which is an RNA molecule.
17. A cell comprising the vector, plasmid, or nucleic acid molecule of any of features 9-16.
18. The cell of feature 17, wherein the cell comprises a genome-integrated viral vector.
19. The cell of feature 17, wherein the cell comprises an episomal form of the vector.
20. A nanoparticle comprising the vector of any of features 9-15, wherein the nanoparticle is capable of delivery of the vector into a CD3+ cell.
21. The nanoparticle of feature 20, wherein the nanoparticle comprises a targeting moiety that promotes selective entry of the nanoparticle into CD3+ cells.
22. The nanoparticle of feature 20 or 21, wherein the nanoparticle is also capable of delivery of the vector into a CD3− cell.
23. The nanoparticle of any of features 20-22, wherein the nanoparticle comprises a polymer.
24. The nanoparticle of feature 23, wherein the polymer is a poly(beta-amino ester).
25. A method of expressing a transgene in a CD3+ cell, the method comprising the steps of:

(a) providing the vector of any of features 9-15, or the nanoparticle of any of features 20-24, and a CD3+ cell, wherein the vector comprises said transgene;

(b) transducing or transfecting the cell with the vector; and

(c) allowing the transgene to be expressed in the transduced or transfected cell.

26. The method of feature 25, wherein the vector is a lentiviral vector.
27. The method of feature 25, wherein the CD3+ cell is CD4+, CD4−, CD8+, or CD8−.
28. The method of feature 25, wherein step (b) comprises contacting the vector with a mixture of CD3+ and CD3− cells, and wherein the CD3+ cells are selectively transduced.
29. The method of any of features 25-28, wherein step (b) is performed in vitro.
30. The method of any of features 25-28, wherein step (b) is performed in vivo and comprises administration of the vector by intravenous, intratumoral, intramedullary, or intraperitoneal injection.
31. A method of making the vector of any one of features 9-15, the method comprising adding the promoter sequence of any of features 1-8 to a vector for use in transducing a CD3+ cell.
32. The method of feature 31, wherein said promoter sequence does not support expression of the transgene in packaging cells or producer cells used to make the vector.

### BRIEF DESCRIPTION OF DRAWINGS

FIGS. 1A, 1B and 1C show in vitro results obtained with lentiviral vectors (LV) encoding green fluorescent protein (GFP) under the control of different human T-cell specific promoters. The transduction efficiency is given for each tested promoter at 72 h post-transduction of HEK293T cells (FIG. 1A), Ramos cells (FIG. 1B), and Jurkat cells (FIG. 1C) analyzed by flow cytometry. Control cells were not transduced (NT) but were kept in culture throughout the experiment and a positive control was obtained by cells transduced with LV carrying the ubiquitous CMV promoter to drive the expression of GFP. For each promoter, 3 different Multiple of Infection (MOI) were evaluated.

FIGS. 2A, 2B and 2C show in vitro results obtained after the transduction of human PBMCs with lentiviral vectors (LV) encoding GFP under the control of different human T-cell specific promoters. The transduction efficiency was measured for each tested promoter at 72 h post-transduction by flow cytometry either with total CD45+ cells (FIG. 2A), CD19+ cells (FIG. 2B) or CD3+ cells (FIG. 2C). Control cells (NT) were not transduced, but were activated and kept in culture throughout the experiment.

FIG. 3A shows the expression level of CD19 CAR on LV293 producing cells 72 h post-transfection with the packaging plasmid (pARA-pack) and the proviral plasmid (pARA-hUBC-CAR-CD19). CD19 CAR expression was measured by flow cytometry with CAR CD19 detection reagent and anti-biotin secondary antibody (α-biotin). Control cells were not transfected with the 2 plasmids and background staining was assessed with the anti-biotin antibody alone.

FIG. 3B shows the expression level of different transgenes (GFP and CD19 CAR) after transduction of PBMCs with PBAE encapsulated VSV-G− (“Bald”) LV or non-encapsulated VSV-G− (“Bald”) LV encoding for GFP or CD19 CAR under the control of ubiquitous promoters CMV and hUBC respectively.

### DETAILED DESCRIPTION

The present technology provides promoter sequences for use in expressing any desired transgene in CD3+ cells. Each of the promoter sequences can be inserted into a vector for expression of a transgene just upstream of the transgene sequence, in the 5′ untranslated region most proximal to the transgene. The promoters are specific for expression in CD3+ cells, which adds an increased level of safety and specificity when using the transgene for gene transfer or immunotherapy, especially for in vivo therapy in which the vector is introduced into a patient.

One aspect of the present technology is a promoter sequence for use in expression of a transgene in a CD3+ cell. The promoter sequence includes at least nucleotides 1501-2000 of any of SEQ ID NOS:2-10 or 12-16 or a variant thereof. Alternatively, the promoter sequence can include nucleotides 1001-2000 of any one of SEQ ID NOS:2-10 or 12-16, nucleotides 501-2000 of any one of SEQ ID NOS:2-10 or 12-16, or nucleotides 1-2000 of any one of SEQ ID NOS:2-10 or 12-16, or a sequence variant of any of these. Further promoter sequences can be derived from any of SEQ ID NOS:2-16, or a variant thereof, by including one or more blocks of nucleotides starting from the 3′ end of the sequence and extending in a 5′ direction; such blocks of nucleotides can contain 200, 300, 400, 500, 600, 700, or more nucleotides, and extending up to a maximum of the first nucleotide of the sequence at the 5′ end. See, for example, SEQ ID NOS:19-29, described in Example 8. Sequence variants as described above can have at least 80%, at least 85%, at least 90%, at least 95%, at least 97%, at least 98%, or at least 99% identity to the stated sequence or its complement.

Another aspect of a promoter sequence of the present technology is that it can contain a binding sequence for one or more transcription factors selected from the group consisting of NF-kappaB, AP-1, STAT, GATA-3, and NFAT, or another transcription factor. The transcription factor binding sites can occur in any combination, in any position, and in any order within the promoter sequence. An online tool for identifying transcription factor binding sites in promoter sequences or putative promoter sequences is available at http://alggen.lsi.upc.es/cgi-bin/promo_v3/promo/promoinit.cgi?dirDB=TF_8.3.

Yet another aspect of a promoter sequence of the present technology is that it is capable of selectively promoting the in vitro or in vivo expression of a transgene in CD3+ cells compared to CD3− cells. For example, the ratio of expression in CD3+ cells to CD3− cells can be at least 2:1, 3:1, 4:1, 5:1, 6:1, 7:1, 8:1, 9:1, 10:1, 15:1, 20:1, or even 50:1 or 100:1, or higher. The promoter sequence can promote the expression of a transgene in CD3+ cells that are either CD4+ or CD4−, in CD3+ cells that are CD8+ or CD8− or in CD3+ cells that are CD4+ CD8+ or CD4− CD8−.

Still another aspect of the technology is a vector containing any of the above-described promoter sequences. The vector can be, for example, a viral vector, such as a lentiviral vector (LV). The vector can also be a “bald” LV, or other viral vector, which lacks a virus fusion protein on the surface of the vector particles, such as described in WO 2019/145796A2, which is incorporated by reference in its entirety herein. Other viral vectors such as retrovirus, adenovirus, adeno-associated virus (AAV), and herpes simplex virus used as gene-delivery vehicles can incorporate the above-described promoter sequences. The vector can be present in a cell, including cells used for immunotherapy or used to produce a recombinant protein. A promoter of the present technology also can be packaged into a plasmid or nucleic acid molecule, such as an RNA molecule, including an mRNA molecule, together with one or more transgenes under its control, for delivery to cells. The vector, plasmid, or nucleic acid molecule can be packaged in a nanoparticle, such as a polymer-containing nanoparticle, including nanoparticles comprising or consisting of polymer-encapsulated vector. Polymers of such nanoparticles can be poly(beta-amino ester)s (PBAEs), including PBAE polymers containing oligopeptide end caps. The vector contains a transgene for expression in a target cell. The transgene can be any gene, including genes intended for immunotherapy involving enhancing the activity of a T-cell or a sub-population of T-cells, including CD4+ cells, CD8+ cells, NK cells, Th cells, or Treg cells.

While the promoters of the present technology can be used to promote the expression of any transgene, the transgene can encode, for example, an anti-checkpoint protein or polypeptide, such as an inhibitor of CTLA-4, PD1, PDL1, LAG-3, TIM 3, B7-H3, ICOS, IDO, 4-1BB, or CD47. The transgene alternatively can encode a chimeric antigen receptor (CAR) having binding specificity for any desired antigen, such as a tumor antigen or an antigen on the surface of a pathogen such as a bacterium, virus, yeast, or parasite. The CAR can be a universal CAR, which binds to an adapter molecule having a domain for CAR binding as well as an antigen binding domain suitable for binding to an antigen such as a tumor antigen or an antigen on the surface of a pathogen such as a bacterium, virus, yeast, or parasite. The transgene can be a protein or a combination of proteins able to elicit an immune response and act as a vaccine. The elicited immune response can be prophylactic or therapeutic, and can stimulate an immune response against bacteria, viruses, other microbial pathogens, or cancer cells, or another undesired cell type found in the body of a subject.

Another aspect of the present technology is a method of expressing a transgene in a CD3+ cell. The method includes the steps of: (a) providing a vector or nanoparticle as described above and a CD3+ cell; (b) transducing the cell, either in vitro or in vivo, with the vector or nanoparticle; and (c) allowing the transgene to be expressed in the transduced cell. The vector or nanoparticles contains the transgene and a promoter as described above in the 5′ untranslated region proximal to the gene.

Even another aspect of the present technology is a method of making the vector described above. The method includes adding any of the above-described promoter sequences to a vector for use in transducing a CD3+ cell.

### EXAMPLES

### Example 1. Production of Lentiviral Vectors Containing Transgenes Under Control of T-Cell Promoters

Different batches of lentiviral vectors (LVs) were prepared and tested in vitro to investigate transgene expression. The LVs were made using the following materials and methods.

Selection of T-Cell Promoters

T lymphocytes represent a diverse population of CD3-positive immune cells, the main types being cytotoxic T-cells (CD8⁺, Tc cells), helper T-cells (CD4⁺, Th cells) and regulatory T-cells (Tregs). The hematopoietic lineage expressing the CD3 receptor also includes immature populations at various stages of differentiation (CD4⁺/CD8⁻, CD4⁻/CD8⁺, CD4+/CD8+) and innate lymphoid cells from which originate natural killer cells (NK cells).

Promoters for specific expression in CD3-positive cells were designed using orthogonal methods. A set of 15 promoters was selected for overexpression in CD3+ cells (SEQ ID NOS:2-16); these promoters are listed in Table 1. Among this group, 3 promoters (SEQ ID NOS:8, 12, and 15) have been described in the literature as partially repressed in Treg and B cells.

Molecular Cloning of T-Cell Promoters

The promoters were ordered as synthetic genes flanked by Mlul and BamHI restriction sites and subcloned into the transfer vector plasmid pARA-CMV-GFP upstream of the open reading frame (ORF) encoding Green Fluorescent Protein (GFP). CMV promoter (SEQ ID NO:1) was chosen as a control as it drives expression of transgenes at a high level in a ubiquitous manner. For SEQ ID NOS:3, 4, and 5, Mlul and BglII sites were chosen as flanking sites due to the presence of BamHI sites within the promoter regions.

The same strategy was followed to subclone the promoters with a T cell specific activity demonstrated in cell assays in a second transfer vector plasmid pARA-hUBC-CAR-CD19 upstream of the ORF encoding a chimeric antigen receptor (CAR) specific for the human CD19 antigen.

TABLE 1
Summary of 15 human promoters selected for expression in CD3-positive cells. SEQ ID NO:, gene function and predicted
expression pattern in the CD3⁺ lymphoid lineage are given. Unknown: data not available in browsed databases.

| | | | Predicted expression pattern within CD3⁺ lymphoid lineage | | | | | | |
|---|---|---|---|---|---|---|---|---|---|
| SEQ | | | | | | | | | Innate |
| ID | | | | | | | | | lymphoid |
| NO: | Promoter | Gene function | CD4⁺ | CD8⁺ | Treg | CD4⁺/CD8⁻ | CD4⁻/CD8⁺ | CD4⁺/CD8⁺ | cells |
| 2 | LAIR2¹ | Leucocyte-associated | + | + | + | Unknown | Unknown | Unknown | + |
| | | Ig-like receptor 2 | | | | | | | |
| 3 | TNFSF8 | Tumor necrosis factor | + | + | + | + | + | + | − |
| | | ligand superfamily | | | | | | | |
| | | member 8 | | | | | | | |
| 4 | UBASH3A | Ubiquitin- associated | + | + | + | + | + | + | + |
| | | and SH3 domain | | | | | | | |
| | | containing A | | | | | | | |
| 5 | ZAP70 | Zeta chain- associated | + | + | + | + | + | + | + |
| | | protein kinase 70 | | | | | | | |
| 6 | BCL11B | Zinc finger protein | Unknown | Unknown | Unknown | Unknown | Unknown | Unknown | Unknown |
| 7 | CTLA4 | cytotoxic T- | + | − | + | + | − | − | − |
| | | lymphocyte-associated | | | | | | | |
| | | protein 4 | | | | | | | |
| 8 | TCF | T-cell specific | + | + | − | + | + | + | − |
| | | transcription factor, | | | | | | | |
| | | HMG-box | | | | | | | |
| 9 | TIGIT | T cell immunoreceptor | + | + | + | − | − | − | + |
| | | with Ig and ITIM | | | | | | | |
| 10 | GIMAP7 | GTPase, IMAP family | + | + | + | + | + | − | + |
| | | member 7 | | | | | | | |
| 11 | TCR² | T cell receptor Vβ 8.1 | Unknown | Unknown | Unknown | Unknown | Unknown | Unknown | Unknown |
| 12 | EOME³ | Eomesodermin | + | + | − | − | − | − | + |
| 13 | ICOS | Inducible T-cell co- | + | + | + | + | + | − | + |
| | | stimulator | | | | | | | |
| 14 | LCK | Lymphocyte specific | Unknown | Unknown | Unknown | Unknown | Unknown | Unknown | Unknown |
| | | protein tyrosine kinase | | | | | | | |
| 15 | ITK⁴ | IL-2 inducible T-cell | + | + | − | + | + | + | + |
| | | kinase | | | | | | | |
| 16 | IL2RA | IL2 receptor alpha | − | − | + | − | − | − | − |

Materials

The transfer vector plasmids were pARA-XXX-GFP and pARA-XXX-CAR-CD19. The kanamycin-resistant plasmids coding for the provirus (a non-pathogenic and non-replicative recombinant proviral DNA derived from HIV-1, strain NL4-3), in which expression cassettes encoding GFP or CAR CD19 were cloned. The insert contained the transgene, the promoter for transgene expression and sequences added to increase the transgene expression and to allow the lentiviral vector to transduce all cell types including non-mitotic ones. The promoters were the human T-cell promoters or the CMV promoter devoid of any enhancer sequence. The non-coding sequences and expression signals corresponded to Long Terminal Repeat sequences (LTR) with the whole cis-active elements for the 5′LTR (U3-R-U5) and the deleted one for the 3′LTR, hence lacking the promoter region (ΔU3-R-U5). For the transcription and integration experiments, encapsidation sequences (SD and 5′Gag), the central PolyPurine Tract/Central Termination Site for the nuclear translocation of the vectors, and the BGH polyadenylation site were added.

The packaging plasmid was pARA-Pack. The kanamycin resistant plasmid encoded for the structural lentiviral proteins (GAG, POL, TAT and REV) used in trans for the encapsidation of the lentiviral provirus. The coding sequences corresponded to a polycistronic gene gag-pol-tat-rev, coding for the structural (Matrix MA, Capsid CA and Nucleocapside NC), enzymatic (Protease PR, Integrase IN and Reverse Transcriptase RT) and regulatory (TAT and REV) proteins. The non-coding sequences and expression signals corresponded to a minimal promoter from CMV for transcription initiation, a polyadenylation signal from the insulin gene for transcription termination, and an HIV-1 Rev Responsive Element (RRE) participating for the nuclear export of the packaging RNA.

The envelope plasmid, when used, was pENV1. This kanamycin-resistant plasmid encoded glycoprotein G from the Vesicular Stomatitis Virus (VSV-G) Indiana strain, used for the pseudotyping of some of the lentiviral vectors. The VSV-G genes were codon optimized for expression in human cells, and the gene was cloned into pVAX1 plasmid (Invitrogen). The coding sequences corresponded to codon-optimized VSV-G gene, and the noncoding sequences and expression signals corresponded to a minimal promoter from CMV for transcription initiation, and the BGH polyadenylation site to stabilize the RNA.

Production of VSV-G⁻ (“Bald”) Lentiviral Vector Particles

LV293 cells were seeded at 5×10⁵ cells/mL in 2×3000 mL Erlenmeyer flasks (Corning) in 1000 mL of LV-Max Production Medium (Gibco Invitrogen). The two Erlenmeyers were incubated at 37° C., 65 rpm under humidified 8% CO₂. The day after seeding, the transient transfection was performed. PEIPro transfectant reagent (PolyPlus Transfection, Illkirch, France) was mixed with transfer vector plasmids (pARA-CMV-GFP or pARA-XXX-GFP or pARA-XXX-CAR-CD19) and packaging plasmid (pARA-Pack). After incubation at room temperature, the mix PEIPro/Plasmid was added dropwise to the cell line and incubated at 37° C., 65 rpm under humidified 8% CO₂. At day 3, the lentivector production was stimulated by sodium butyrate at 5 mM final concentration. The bulk mixture was incubated at 37° C., 65 rpm under humidified 8% CO₂ for 24 hours. After clarification by deep filtration at 5 and 0.5 μm (Pall Corporation), the clarified bulk mixture was incubated 1 hour at room temperature for DNase treatment.

Lentivector purification was performed by chromatography on a Q mustang membrane (Pall Corporation) and eluted by NaCl gradient. Tangential flow filtration was performed on a 100 kDa HYDROSORT membrane (Sartorius), which allowed to reduce the volume and to formulate in specific buffer at pH 7, ensuring at least 2 years of stability. After sterile filtration at 0.22 μm (Millipore), the bulk drug product was filled in 2 mL glass vials with aliquots less than 1 ml, then labelled, frozen and stored at <−70° C.

The bald LV number was evaluated by physical titer quantification with two methods: p24 ELISA and qRT-PCR. The p24 ELISA was performed by detection and quantitation of the lentivirus associated HIV-1 p24 core protein only (Cell Biolabs Inc.). A pre-treatment of the samples allows to distinguish the free p24 from destroyed Lentivectors. The qRT-PCR was performed by purification of lentiviral RNA with Nucleospin RNA virus kit (Macherey Nagel) and quantitation with Lenti-X qRT-PCR titration kit (Takara). For each LV batch, physical titer, particle size and size distribution were measured by Nanoparticle Tracking Analyzer and Dynamic Light Scattering (Viewsizer 3000 and NanoPartica SZ-100V2 instruments respectively, Horiba Instruments Inc., USA) Analyses were performed at room temperature after dilution of LV in formulation buffer (10-fold for DLS and 300-fold for NTA) but without filtration not to impact biophysical properties of the samples. The results were determined using the control Horiba softwares of the equipments.

Production of VSV-G⁺ (“Pseudotyped”) Lentiviral Vector Particles

The same above-described method was used except that PEIPro transfectant reagent (PolyPlus, 115-010) was mixed with transfer vector plasmid (pARA-CMV-GFP or pARA-XXX-GFP or pAra-XXX-CAR-CD19), packaging plasmid (pARA-Pack) and the envelope plasmid (pENV1). Titration for the pseudotyped Lentivectors was performed by quantitative PCR three days post-transduction of HEK293T cells (8×10⁵ cells/well) with clarified bulk obtained during the bioproduction.

As shown in Table 2, production yields were generally as effective as those obtained with the CMV promoter despite the fact that these promoter regions were significantly longer. For all constructs, titers were at least in the 10⁷ TU/mL range and were consistent among different production runs. The lowest titers were obtained with promoters based on SEQ ID 4 and 8. Yields of production are a criteria for the selection of the promoters, as a low yield implies future difficulties for industrialization. In any case, no issue that would impact industrial bioproduction was observed.

TABLE 2
Summary of production yields obtained for pseudotyped LV coding
for GFP under the control of 14 human promoters identified by
a bioinformatics approach to be expressed in CD3-positive cells.

| | | Size of | | |
|---|---|---|---|---|
| | | promoter | Gene of | Infectious titer |
| SEQ ID | Promoter | region (bp) | interest | qPCR (TU/mL) |
| 1 | CMV | 776 | GFP | 2.38 × 10⁷ |
| 2 | LAIR2 | 2000 | GFP | 2.61 × 10⁷ |
| 3 | TNFSF8 | 2000 | GFP | 2.63 × 10⁷ |
| 4 | UBASH3A | 2000 | GFP | 1.05 × 10⁷ |
| 5 | ZAP70 | 2000 | GFP | 3.93 × 10⁷ |
| 6 | BCL11B | 2000 | GFP | 2.99 × 10⁷ |
| 7 | CTLA4 | 2000 | GFP | 1.73 × 10⁷ |
| 8 | TCF | 2000 | GFP | 1.18 × 10⁷ |
| 9 | TIGIT | 2000 | GFP | 4.44 × 10⁷ |
| 10 | GIMAP7 | 2000 | GFP | 4.49 × 10⁷ |
| 11 | TCR | 781 | GFP | 3.54 × 10⁷ |
| 12 | EOME | 2000 | GFP | 1.56 × 10⁷ |
| 13 | ICOS | 2000 | GFP | 2.16 × 10⁷ |
| 14 | LCK | 2000 | GFP | 6.00 × 10⁷ |
| 15 | ITK | 2000 | GFP | 1.72 × 10⁷ |

### Example 2. Transduction of HEK293T, Ramos, and Jurkat Cells by Lentiviral Vectors Containing Transgenes Under Control of T-Cell Promoters

In order to investigate the CD3-specific activity of the 15 promoters identified by bioinformatic tools, we used pseudotyped Lentiviral Vectors carrying a green fluorescent protein (GFP) transgene under the control of a given promoter to transduce different human cell types and analyze GFP expression. These studies were performed on CD3-positive Jurkat (Acute T Cell Leukemia Human Cell Line—ATCC TIB-152), CD3-negative Ramos (Burkitt's Lymphoma Human Cell Line—ATCC CRL-1596) and non-lymphocyte HEK293T (Human Embryonic Kidney Cell Line—ATCC CRL-1573).

HEKT293 cells were seeded in 24-well plates at a density of 8×10⁴ cells per well in DMEM medium (Gibco Invitrogen) supplemented with 10% FBS (Gibco Invitrogen), 1 penicillin/streptomycin and incubated for 4 h to adhere. Cells were then transduced by replacing the medium with 300 μL of Lentiviral Vector (at a MOI of 1, 5 or 10) in culture medium or culture medium (NT controls), followed by incubation at 37° C., 5% CO₂ for 2 h. After adsorption, 1 mL of complete medium was added to each well. At 72 h post transduction, the cells were trypsinized and resuspended in 200 μL of Cellfix 1×, and the percentage of cells expressing GFP was determined with an Attune N×T flow cytometer (ThermoFisher) using the BL1 channel.

Jurkat and Ramos cells were seeded in 24-well plates at a density of 8×10⁴ cells per well in RPMI-1640 medium (Gibco Invitrogen) supplemented with 10% FBS (Gibco Invitrogen), 1% penicillin/streptomycin. Cells were then transduced by replacing the medium with 300 μL of Lentiviral Vector (at a Multiple of Infection (MOI) of 10, 30 or 50) in culture medium or culture medium alone (NT controls). After 2 h incubation at 37° C., 5% CO₂, 500 μL of fresh complete medium was added to each well. The percentage of cells expressing GFP transgene was determined 72 h post-transduction with an Attune N×T flow cytometer using the BL1 channel.

The percentages of viable and GFP-positive cells were determined by gating on debris excluded/viable/single cells. Graphed data represent means of triplicates of a representative experiment.

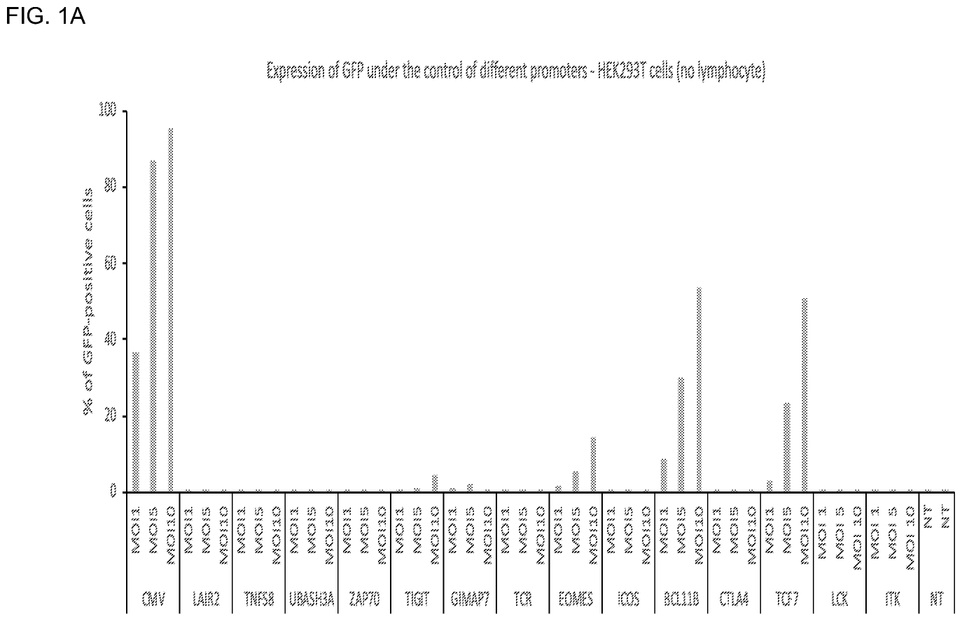

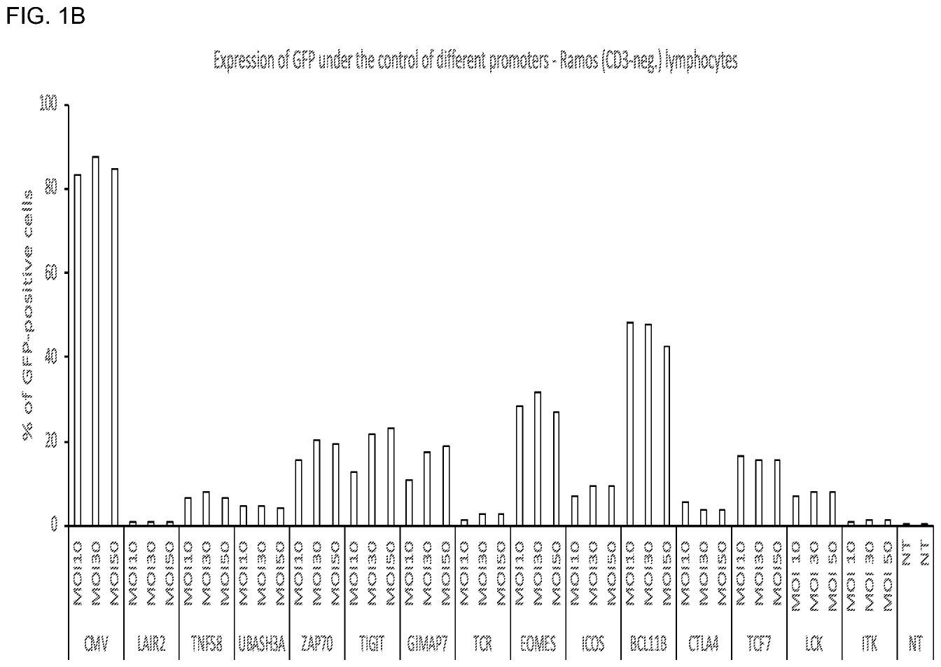

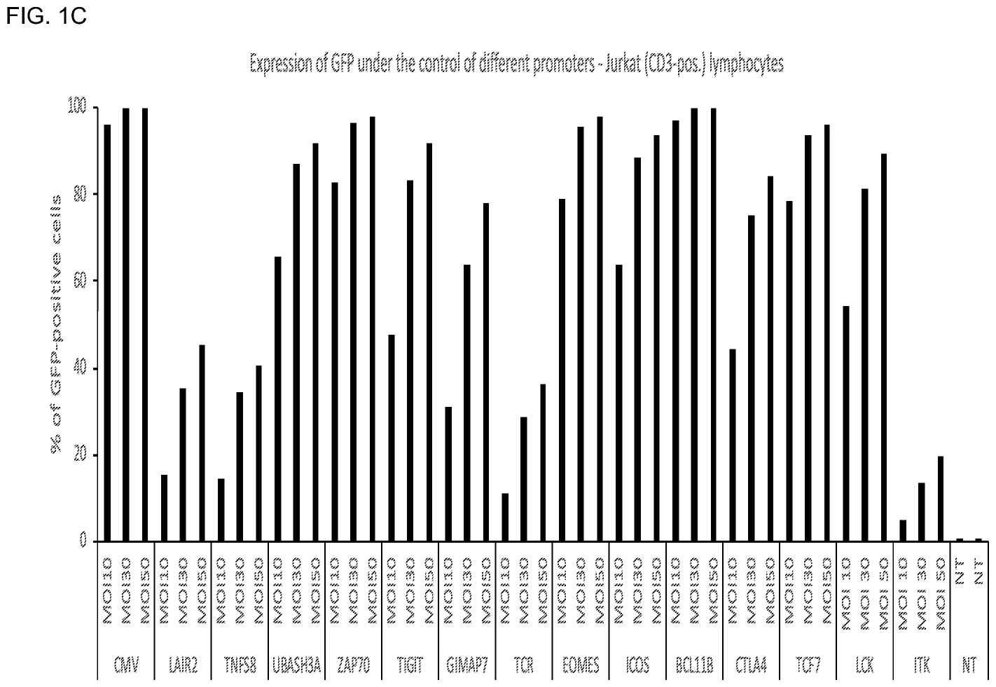

The results presented in FIG. 1A (HEK293T cells), 1B (Ramos CD3− cells) and 1C (Jurkat CD3+ cells) show that, for all cell types, differences in expression patterns were observed between the promoters. Although lower than levels observed with ubiquitous CMV promoter, significant GFP expression (above 10%) was measured in human CD3-negative lymphocytes (Ramos cells) and non-lymphocyte cells (HEK293T) at the 3 tested MOI with promoters ZAP70 (SEQ ID NO: 5), BCL11B (SEQ ID NO: 6), TCF7 (SEQ ID NO: 8), TIGIT (SEQ ID NO: 9), GIMAP7 (SEQ ID NO: 10) and EOMES (SEQ ID NO: 12) ruling out any specificity for CD3-positive cells.

Promoters TNFS8 (SEQ ID NO: 3), UBASH3 (SEQ ID NO: 4), CTLA4 (SEQ ID NO: 7), ICOS (SEQ ID NO: 13) and LCK (SEQ ID NO: 14) drove the highest GFP expression (above 40% at MOI 10) in CD3-positive Jurkat cells at levels comparable with CMV promoter. However, background GFP expression (5-10% GFP-positive cells) was detected in CD3-negative cells with these promoters. A dose-dependent and strictly restricted to CD3-positive Jurkat cells GFP expression was achieved with promoters LAIR2 (SEQ ID NO: 2), TCR (SEQ ID NO: 11) and ITK (SEQ ID NO: 15). The CD3-specific activity of these last three promoters resulted in weaker GFP expression with maximal values of 45% reached at MOI of 50.

Of note, none of the tested promoters had an impact on viability of transduced cells (data not shown).

### Example 3. Transduction of Human PBMCs Using Lentiviral Vectors Containing Transgenes Under Control of T-Cell Promoters

Purification of Human PBMCs

Peripheral Blood Mononuclear Cells (PBMCs) were isolated from buffy coats obtained from healthy donors (Etablissement Francais du Sang, Division Rhones-Alpes). After diluting the blood with DPBS, the PBMCs were separated over a FICOLL density gradient (GE Healthcare) and washed twice with DPBS. Then, residual Red Blood Cells were lysed during a 5 min incubation of PBMCs in ACK lysis buffer (Gibco) and an additional DPBS washing. PBMCs were frozen at a density of 20×10⁶ cells/mL in 10% DMSO (Sigma), 90% FBS (Gibco) and stored at −150° C. until use.

Activation and Transduction of PBMCs with VSV-G⁺ (“Pseudotyped”) Lentiviral Vector

The CD3-specific activity of the six promoters screened in Example 2 was evaluated in human quiescent cells with LV carrying a GFP transgene under the control of a given promoter to transduce previously frozen human PBMCs. Thawed human PBMCs were seeded in 24-well plates at a density of 1×10⁶ cells per well in RPMI medium containing 10% FBS (Gibco) and 1% penicillin/streptomycin (Gibco), activated in presence of CD3-CD28 Dynabeads (Gibco) and incubated for 72 h at 37° C. and 5% CO₂. Activated PMBCs were then pooled and transduced with encapsulated pseudotyped LV (at a MOI of 20, 50 or 100) in culture medium or culture medium alone (NT controls) in 24-well plates at a density of 1×10⁵ cells. After 2 h incubation at 37° C., 5% CO₂, 500 μL of fresh culture medium (Gibco Invitrogen) was added to each well and incubated for 72h. The percentage of cells expressing GFP was determined 72 h post-transduction with an Attune N×T flow cytometer. The phenotype of transduced cells expressing GFP transgene was determined by flow cytometry staining with antibodies specific for the following cell types following manufacturer's instructions (Biolegend): CD3-AF700, CD14-PE-Cy7, CD16-BV711, CD19-BV605, CD45-BV510, CD56-BV421 and Zombie NIR for live/dead discrimination. After 30 min incubation at 4° C., cells were centrifuged at 500×g for 2 min and fixed with CelIFix solution (BD Biosciences). Fluorescence-positive cells were counted by flow cytometry (AttuneNXT; Invitrogen, Inc.) on BL1 (GFP), RL2 (AF700 dye), RL3 (Zombie NIR), VL1 (BV421 dye), VL2 (BV510 dye), VL3 (BV605 dye), VL4 (BV711 dye) and YL4 (PE-Cy7 dye) channels. Cell phenotypes were defined for CD45+, viable and single cells as follows: T lymphocytes (CD3^{pos}-CD19^{neg}), B lymphocytes (CD3^{neg}-CD19^{pos}), NK cells (CD3^{neg}-CD19^{neg}-CD56^{pos}), monocytes (CD14^{pos}) and granulocytes (SSC^{high}-CD16^{pos}).

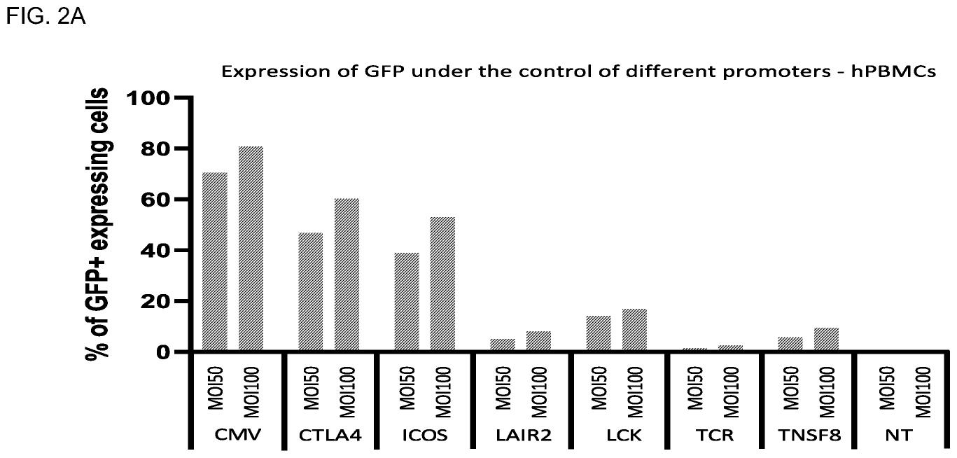

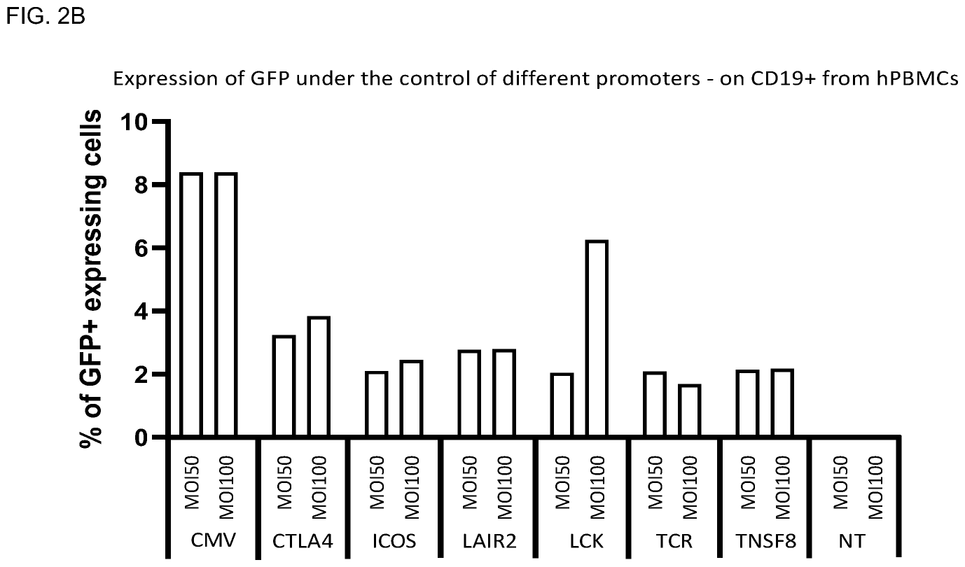

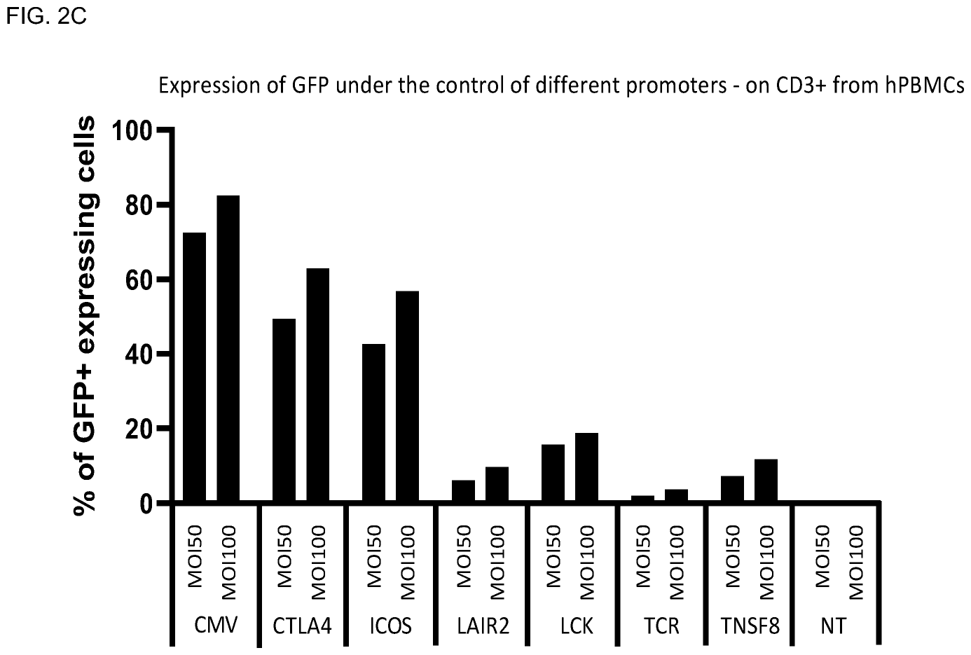

The results presented in FIG. 2A (whole CD45+ PBMCs), 2B (CD19+ cells among PBMCs) and 2C (CD3+ cells among PBMCs) show the differences in the GFP expression patterns obtained with the tested promoters. The highest GFP expression (above 40%) was measured on total CD45+ PBMCs and among gated CD3+ T cells at the 2 tested MOI with promoters CTLA4 (SEQ ID NO: 7) and ICOS (SEQ ID NO: 13). However, GFP expression was weaker in CD3+ primary cells than on CD3+ cell lines for LAIR2 (SEQ ID NO: 2), LCK (SEQ ID NO: 14), TCR (SEQ ID NO: 11) and TNFS8 (SEQ ID NO: 3) (below 20%). In addition, only LCK (SEQ ID NO: 14) at MOI 100 was shown to induce GFP expression in CD19+ cells at the same level as for CMV. The other selected promoters resulted in weaker GFP expression than CMV, which confirms their CD3+ selectivity of both cell lines and primary samples. Finally, none of the tested promoters had an impact on viability of transduced primary cells (data not shown).

### Example 4. Transduction of Primary Lymphocytes and PBMCs Using PBAE-Encapsulated VSV-G− (“Bald”) LV

The CD3-specific activity of the promoters screened in Examples 2 and 3 is evaluated in human quiescent cells with PBAE-encapsulated bald LV carrying a GFP transgene under the control of a given promoter to transduce human PBMCs. PBMCs are isolated from buffy coats obtained from healthy donors (Etablissement Francais du Sang, Division Rhones-Alpes) or from blood samples from lymphoma patients as purified and frozen cells available at Lonza and CALYM Network (Centre Hospitalier Lyon-Sud, France) Biobanks.

Purification of Human PBMCs

After diluting the fresh blood with DPBS, the PBMCs are separated over a FICOLL density gradient (GE Healthcare) and washed twice with DPBS. Then, residual red blood cells are lysed during a 5 min incubation of PBMCs in ACK lysis buffer (Gibco) and an additional DPBS washing. PBMCs are frozen at density of 20×10⁶ cells/mL in 10% DMSO (Sigma), 90% FBS (Gibco) and stored at −150° C. until use.

Transduction of PBAE-Encapsulation of VSV-G− (“Bald”) Lentiviral Vector

Because non-dividing cells are generally difficult to transduce with LV (without cytokine and CD3-CD28 activation), oligopeptide-modified poly(beta-amino ester) (OM-PBAE) polymers are used as transfection agents instead. OM-PBAEs have already been described as transfection agents that form polymer-encapsulated vehicles able to deliver genetic material (plasmids or other nucleic acid molecules) to eukaryotic cells (US2016/0145348A1, Mangraviti et al. 2015, Anderson et al. 2004, WO2016/116887). OM-PBAEs have been successfully used to coat transduction-deficient lentiviral vectors and engineer human cells to stably express various transgenes including reporter genes (Green Fluorescent Protein -GFP and mCherry) and CARs (see WO2019/145796). The polymers used in the following encapsulation experiments are poly(beta-amino esters) (PBAEs) conjugated to charged peptides. Polymer PBAE-CR3 refers to PBAE conjugated to the peptide CRRR (SEQ ID NO:17 (same peptide at both ends). PBAE-CH3 polymer refers to PBAE conjugated to the peptide CHHH (SEQ ID NO:18). Mixtures of these OM-PBAEs are tested at a 60/40 molar ratio.

Human PBMCs are seeded in 24-well plates at a density of 1×10⁵ cells per well in RPMI medium containing 10% FBS and 1% penicillin/streptomycin. Cells are then transduced by replacing the medium with 100 to 300 μL of encapsulated pseudotyped LV in culture medium or culture medium alone (NT controls). After 2 h incubation at 37° C., 5% CO₂, 600 μL of fresh complete medium are added to each well. The percentage of cells expressing GFP is determined 72 h post-transduction with an Attune N×T flow cytometer using the BL1 channel. The phenotype of transduced cells expressing GFP transgene is determined by flow cytometry staining with antibodies specific for the following cell types following manufacturer's instructions (Biolegend): CD3-AF700, CD14-PE-Cy7, CD16-BV711, CD19-BV605, CD45-BV510, CD56-BV421 and Zombie NIR for live/dead discrimination. After 30 min incubation at 4° C., cells are centrifuged at 500×g for 2 min and fixed with CelIFix solution (BD Biosciences). Fluorescence-positive cells are counted by flow cytometry (AttuneNXT; Invitrogen, Inc.) on BL1 (GFP), RL2 (AF700 dye), RL3 (Zombie NIR), VL1 (BV421 dye), VL2 (BV510 dye), VL3 (BV605 dye), VL4 (BV711 dye) and YL4 (PE-Cy7 dye) channels. Cell phenotypes are defined for CD45+, viable and single cells as follows: T lymphocytes (CD3pos-CD19neg), B lymphocytes (CD3 neg-CD19pos), NK cells (CD3neg-CD19neg-CD56pos), monocytes (CD14pos) and granulocytes (SSChigh-CD16pos).

Finally, this set of experiments is carried out with the promoters that showed the most stringent CD3-specific activity for the expression of intracellular GFP but in the context of transgenes expressed on the surface of lymphocytes, LV without the VSV-G envelope and carrying an anti-CD19 CAR under the control of a given promoter are used to transduce PBMCs from healthy donors and lymphoma patients. The percentage of cells expressing CAR CD19 is determined 72 h post-transduction by flow cytometry with a human CD19 detection reagent (Miltenyi) and an anti-biotin-BB515 antibody (Miltenyi) according to manufacturer instructions using the BL1 channel.

### Example 5. In Vivo Transduction of Peripheral Blood Mononuclear Cells with VSV-G⁻ (“Bald”) Lentiviral Vector Particles Encapsulated in OM-PBAEs

The CD3-specific activity of the 2 best promoters confirmed in Example 4 to trigger the expression of GFP in lymphocytes is evaluated in vivo in immunodeficient NSG mice engrafted with human PBMCs from healthy donors or PBMCs from lymphoma patients. Another mouse model which can be used is NSG mice engrafted with human CD34-positive hematopoeitic stem cells that exhibit multi-lineage engraftment of human immune cell populations. Nanoparticles described in Example 4 consisting in LV carrying a GFP transgene under the control of a given promoter and formulated with and without PBAEs covalently linked with CD3 targeting agents are repeatedly injected intravenously in mice that have previously been injected with PBMCs or CD34-positive hematopoietic stem cells. Control animals are injected with vehicle or transduction-deficient lentiviral vectors lacking the VSV-G envelope and not encapsulated in PBAE polymers.

In vivo biodistribution of expressed GFP transgene is evaluated weekly over 24 to 90 days on whole blood cells by flow cytometry staining with specific antibody panels purchased from Biolegend: general panel (CD3-AF700, CD11b-APC, CD11c-PE, CD14-PE-Cy7, CD16-BV711, CD19-BV605, CD45-BV510, CD56-BV421, CD66b-PerCP-Cy5.5, HLA-DR PE-Dazzle594, and Zombie NIR) and T cell panel (CD3-AF700, CD4-PerCP-Cy5.5, CD8-BV605, CD25-PE, CD45-BV510, CD45RA-BV711, CD69-PE-Dazzle594, CD127-APC, TCRg/d-PE-Cy7, CCR7 (CD197)-BV421 and Zombie NIR).

At sacrifice, the phenotype of GFP-expressing cells is determined on blood cells and cell suspension prepared from collected spleens and bone marrows. Tissue biodistribution of genome-integrated lentiviral vector is analyzed by duplex quantitative PCR on genomic DNA extracted from blood and organs collected at sacrifice. Treatment toxicity is evaluated by determining blood cell counts (flow cytometry as already described in Example 2), ALT/AST hepatic enzymes (enzyme activity kits) and cytokine levels (Th1/Th2 Cytometry Bead Array) in blood samples collected pre-treatment and at weekly post-treatment intervals. Behavior of animals, body weight, water and food consumption are recorded 3 times a week as additional read-outs for treatment safety and tolerance.

Finally, this set of animal experiments is carried out with the promoters selected in Example 4 but in the context of an anti-CD19 CAR transgene expressed on the surface of lymphocytes. The only difference with the above-described animal procedures lies in the evaluation of the phenotype of CD19 CAR-expressing cells that is determined by flow cytometry with the human CD19 detection reagent (Miltenyi) and an anti-biotin-BB515 antibody (Miltenyi) according to manufacturer instructions already described in Example 4.

### Example 6. Efficacy of In Vivo Transduction of Peripheral Blood Mononuclear Cells with VSV-G⁻ (“Bald”) Lentiviral Vector Particles Encapsulated in OM-PBAEs

The CD3-specific activity of the 2 best promoters screened in Example 4 to trigger the expression of CAR CD19 in lymphocytes is evaluated in vivo in immunodeficient NSG mice bearing Ramos cancer cells modified to constitutively express the Luciferase reporter gene. Different NSG mice models are used: NSG mice engrafted with human PBMCs from healthy donors or PBMCs from lymphoma patients and finally NSG mice engrafted with human CD34-positive hematopoeitic stem cells that exhibit multi-lineage engraftment of human immune cell populations.

Nanoparticles described in Example 4 consisting of LV carrying a CAR CD19 transgene under the control of a given promoter and formulated with and without PBAEs covalently linked with CD3 targeting agents are repeatedly injected intravenously in mice that have previously been injected with Ramos-Luc cells. Control animals are injected with vehicle or transduction-deficient LV lacking the VSV-G envelope and not encapsulated in PBAE polymers.

In vivo efficacy is evaluated weekly over 24 to 90 days by whole animal bioluminescence imaging to measure the tumor growth and rate associated with circulating

Ramos-Luc cells and survival in the treated groups versus controls. Treatment toxicity is evaluated by determining blood cell counts (flow cytometry as already described in Example 5), ALT/AST hepatic enzymes (enzyme activity kits) and cytokine levels (Cytometry Bead Array) in blood samples collected pre-treatment and at weekly post-treatment intervals. Behavior of animals, body weight, water and food consumption are recorded 3 times a week as additional read-outs for treatment safety and tolerance.

### Example 7. Design of T-Cell Specific Chimeric Promoters

The promoter sequences described above contain some transcription factor binding sites, including NFκB; AP1, Stats, GATA and NFAT. Table 3 summarizes the presence of those binding sequences inside each promoter sequence. Transcription factor binding sites are depicted in the sequences as well.

TABLE 3
Mapping of the promoter sequences with T-cell specific transcription
factor binding sites. Indicated in italics are the promoters
driving an unspecific expression of the transgene (according
to the experiments described in FIG. 1).

| Promoter | NFκB | AP-1 | STATS | GATA-3 | NFAT |
|---|---|---|---|---|---|
| EOMES | 3 | 4 | 13 | 3 | 13 |
| LCK | 2 | 6 | 6 | 2 | 6 |
| BCL11B | 3 | 3 | 6 | 2 | 4 |
| ZAP70 | 3 | 5 | 6 | 1 | 7 |
| ICOS | 5 | 7 | 8 | 4 | 10 |
| UBASH3A | 7 | 4 | 15 | 7 | 0 |
| GIMAP7 | 2 | 4 | 4 | 5 | 12 |
| LAIR2 | 1 | 7 | 8 | 1 | 5 |
| CTLA4 | 3 | 4 | 5 | 2 | 6 |
| ICR2A | 8 | 1 | 7 | 1 | 5 |
| TCR | 2 | 1 | 3 | 1 | 2 |
| TIGIT | 5 | 5 | 6 | 4 | 6 |
| TCF7 | 1 | 6 | 6 | 1 | 1 |
| TNFS8 | 2 | 4 | 9 | 6 | 7 |
| ITK | 2 | 4 | 12 | 8 | 8 |

The CD3+ specific promoters (LAIR2, TNFS8, TCR and ITK) present less than 2 NFκB sites, less than 8 NFAT boxes and less than 8 NFκB + AP1 sites.

### Example 8: Optimization and Shortening of Promoter Sequences

To define the significant regions involved in inducible gene expression and increased transgene expression level^{5,6,7}, 5′ deletion constructs were designed by shortening of 2 kb-promoters.

Shortening and Molecular Cloning of Tcell Promoters

Among the set of 15 promoters, CTLA4 (SEQ ID NO: 7) and ICOS (SEQ ID NO: 13) were selected for overexpression in CD3-expressing cell lines and primary cells, and low or no expression in non-CD3-expressing cells. The promoters were shortened from 5′ to 3′ by PCR using the synthetic gene as template and Phusion HF high fidelity DNA polymerase (Thermofisher). Forward primers, containing a Mul restriction site, were specific of 5′ regions of CTLA4 and ICOS and reverse primers, containing a BglII site (for SEQ ID NOS: 24-25) or a BamHI site (for SEQ ID NOS: 19-23, 26-29) were specific to the 3′ end of CTLA4 or ICOS. PCR fragments were then subcloned in the transfer vector plasmid pARA-CMV-GFP upstream of the open reading frame (ORF) encoding GFP and instead of the CMV promoter.

The same strategy was followed to subclone the truncated promoters in a second transfer vector plasmid pARA-hUBC-CAR-CD19 upstream of the ORF encoding a chimeric antigen receptor (CAR) specific for the human CD19 antigen. Sequence identity of truncated promoters and in frame insertion upstream of the transgene were verified by Sanger sequencing. Resulting optimized promoter sequences are described in Table 4.

TABLE 4
Shortened CTLA4 and ICOS Promoters.

| | | Size of promoter |
|---|---|---|
| SEQ ID | Promoter | region (bp) |
| 19 | CTLA4_1 | 1844 |
| 20 | CTLA4_2 | 1237 |
| 21 | CTLA4_3 | 1048 |
| 22 | CTLA4_4 | 745 |
| 23 | CTLA4_5 | 344 |
| 24 | ICOS_1 | 1703 |
| 25 | ICOS_2 | 1443 |
| 26 | ICOS_3 | 1219 |
| 27 | ICOS_4 | 1000 |
| 28 | ICOS_5 | 837 |
| 29 | ICOS_6 | 550 |

LV Production and Transduction of Human Cell Lines and Primary Cells

Transfer plasmids encoding GFP and a CD19-targeted CAR were used to produce LV and transduced cell lines and primary cells as described in Examples 3 and 4. GFP or CD19 CAR transgene expression were measured by flow cytometry.

### Example 9: “Pseudotyping” of Lentiviral Vectors with CARs

The experiments of Examples 3 and 4 showed that bald LV packaged into nanoparticles, such as by OM-PBAE encapsulation, have the ability to transduce PBMCs, including T lymphocytes, and direct the expression of a transgene. Since the goal of CAR T cell therapy is to introduce a CAR into T cells and express the CAR on the surface of such T cells, the question arises whether the presence of any expressed CAR on the surface of LV intended for use in producing CAR T cells can serve to pseudotype the LV, and possibly direct the LV to transduce cells that were intended for attack by CAR T cells. The experiments described below were designed to address this question.

Production of Lentiviral Vector Particles Coding for CD19 CAR

Production of bald LV particles was performed as described in Example 1. Briefly, LV293 cells were transiently transfected the day after seeding. PEIPro transfectant reagent (PolyPlus Transfection, Illkirch, France) was mixed with the transfer vector plasmid pARA-hUBC-CAR-CD19 and the packaging plasmid pARA-Pack. Bald LV particles encapsulated in OM-PBAEs were prepared as described in Example 3 and used to transduce LV293 cells or human PBMCs.

CD19 CAR Detection

After a DPBS washing step, cells (LV293 transfected and untransfected or LV-transduced PBMCs) were stained with human CD19 detection reagent (Miltenyi) and an anti-biotin-BB515 antibody (Miltenyi) according to manufacturer instructions as already described in Example 4.

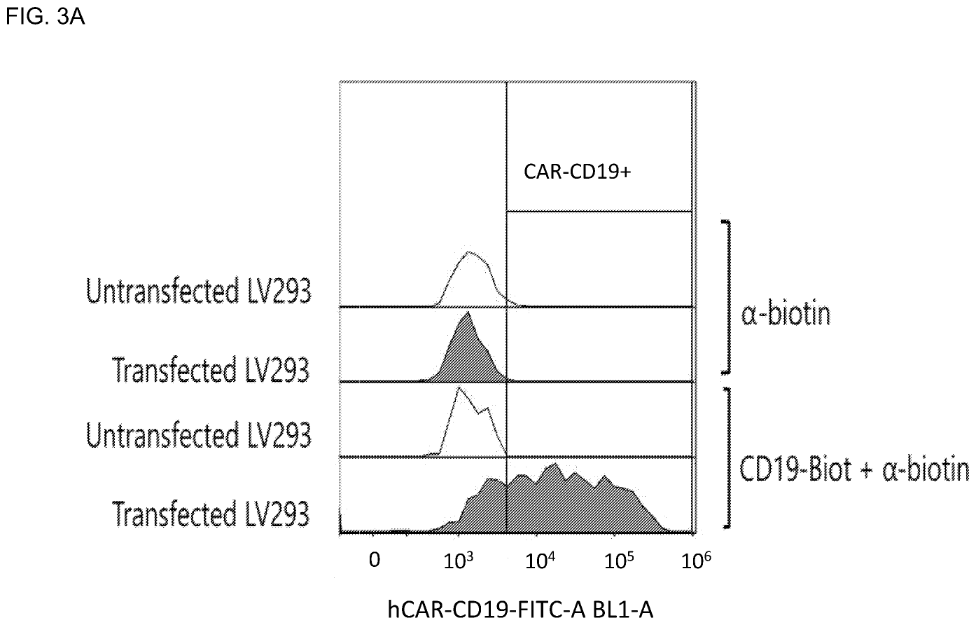

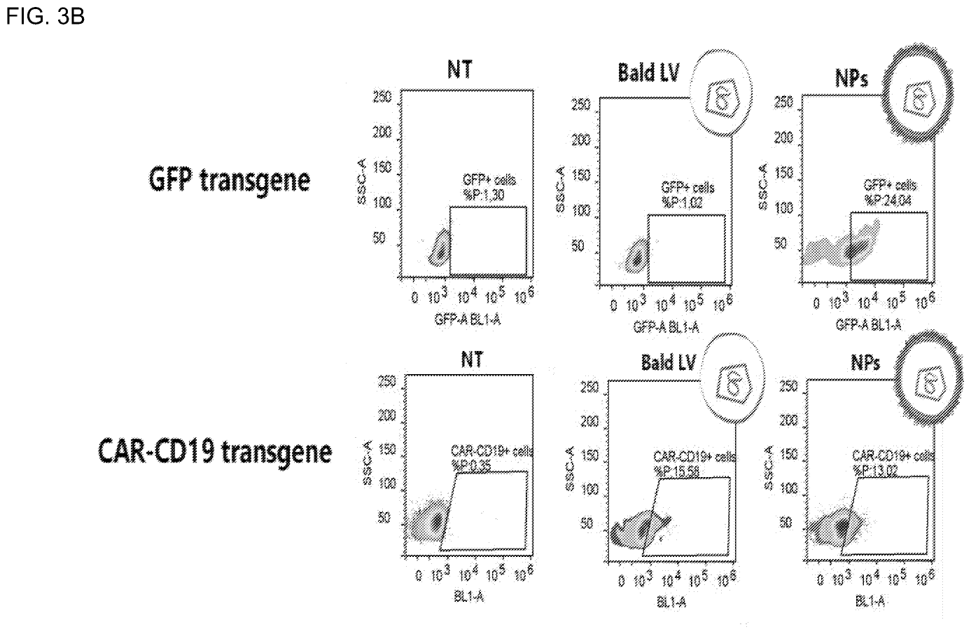

The results presented in FIG. 3A show CD19 CAR expression on the surface of LV293 cells used for the production of LVs (transfected with pARA-hUBC-CAR-CD19 and pARA-Pack plasmids). Untransfected cells were used as control. A strong expression of the CD19 CAR was detected only on the surface of LV293-producing cells after transfection.

In addition, results presented in FIG. 3B show expression obtained on human PBMCs transduced with VSV-G− (“Bald”) LV encoding CD19 CAR under the control of hUBC promoter or GFP under the control of CMV promoter. While no expression of GFP was observed in PBMCs exposed to bald LVs, their encapsulation in OM-PBAE polymers restored their transduction efficiency. When VSV-G− (“Bald”) LV encoding CD19 CAR were used to transduce PBMCs, similar expression levels of the chimeric receptor were observed in presence or absence of the MO-PBAE polymers.

Altogether, these results suggest that the use of a ubiquitous promoter to control the expression of CD19 CARs results in the expression of chimeric receptor on the surface of the LV293 cell line that produces LVs (bald or pseudotyped) decorated with membrane proteins. This pseudotyping with CD19 CARs must be sufficient to allow the bald vector to bind to cells and generate an artefactual CD19-CAR signal, because the bald LV are deficient for transduction by nature, even though the CD19-CAR is not an efficient pseudotyping protein, as it does not allow endosomal escape of the LV). This “pseudotyping” may lead to safety issues, as the regular LV (with VSV-G) will directly target CD19+B lymphocytes both in vitro and in vivo. The results obtained here indicate that use of a T-cell specific promoter as described herein will abrogate the expression of CD19 CAR at the surface of LV293, and no pseudotyping of LV will be observed. This will increase the safety margin of LV designed for producing CAR T cells.

### Sequences

Transcription factor binding sites are depicted in each promoter sequence as follows: NFkB (bold), AP-1 (underlined), STATS (italics), GATA-3 (double-underlined) and NFAT 5 (boxed). Of note, some binding sites are overlapping.

SEQ ID NO: 1
GGAGTTCCGCGTTACATAACTTACGGTAAATGGCCCGCCTGGCTGACCGCCCAACGACCCCC
GCCCATTGACGTCAATAATGACGTATGTTCCCATAGTAACGCCAATAGGGACTTTCCATTGA
CGTCAATGGGTGGAGTATTTACGGTAAACTGCCCACTTGGCAGTACATCAAGTGTATCATAT
GCCAAGTACGCCCCCTATTGACGTCAATGACGGTAAATGGCCCGCCTGGCATTATGCCCAGT
ACATGACCTTATGGGACTTTCCTACTTGGCAGTACATCTACGTATTAGTCATCGCTATTACC
ATGGTGATGCGGTTTTGGCAGTACATCAATGGGCGTGGATAGCGGTTTGACTCACGGGGATT
TCCAAGTCTCCACCCCATTGACGTCAATGGGAGTTTGTTTTGGCACCAAAATCAACGGGACT
TTCCAAAATGTCGTAACAACTCCGCCCCATTGACGCAAATGGGCGGTAGGCGTGTACGGTGG
GAGGTCTATATAAGCAGAGCTCGTTTAGTGAACCGTCAGATC
SEQ ID NO: 2
CCCACTTGCTCCCTGGGCTTGGAGCACAACCACTCAAACAGAACTGGCTTTTGGTCAGTAAG
GAAGAAGTGAGCAACGGCTGCGGTGTAGACCCTCGTCAATGCCTGCGACGGTTACACCTGGA
GACAAGCTCCCCAGTGTCCTCAGGAGCAGCGGAGATGAGAATCCATGATAGGGTGGGCTCTG
TCCCCCTCAGCTCCGTGATGCCGAAATGCACTGCTGGTCCTGGTCCTGCTCCTCATTCCACA
CCCGGCTGAGTGCCCATCTGACCCCAGACCTCAACGCGAGGTTCTAAGCACTGTCTCCTGAC

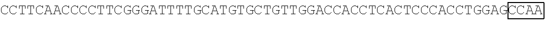

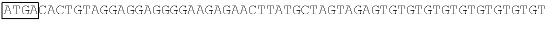

GTGTGTGTGAATGTGTGTGTGTATTATACATAATATATATAATTACAACATTGTTAATGGGG
CCGGGCGCGGTGGCTCACACCTGTAATCACAGCACTTTGGGAGGCTGAGATGGGCAGATCAC
ATGAGGTCAGGAGTTCAAGACCAGCCTGGCCAACATGGTGAAACCCCGTCTCTACTAAAATA
CAAAAATTAGCCGGGCATGGTGGCGTGCGCCTGTAGTCCCGGCTACTCAGCAGGCTGAGGCA
AGAGAATTGCTTGAGCCTGGGAGGCGGAGGTTGCAGTGAGCCAAGATTGCACCACTGCACTC
CAGCCTGGGCAACAGAGTACGACTCCATCTCCACACACACATACACACACACACACACACAC
ACAAATTGTTAATTGTATATGTATAATTATAATAGTTTATATATATTAACAATTATATATAA
ACTTGCATATATATAGTATTTGCTGTATTATTATATATAAACAATTATATATGTAATGACTG
TATAAAATAGATAAACAATTTTAACTAATAATATTATATTAATTATATTATTATTAGATGTA
ATAATTATAATTATTTATATATAATATTTATTATTATGTATCATTGTTAGAACACTTAACGT
GAGCTCTGTCCTCTTAACAAATTTCAAGTGAACAAGACGTTATTGCTGACGATGGGTCGTAT

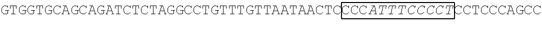

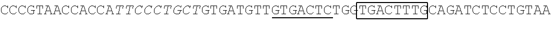

GTGACATCATGCAGTACTTGGTCTCTGCCTCTGCGTCGCTTGGCGTGATGTCCTCAGGTTTC
GTCCGTGTTGTCGCCCATGGCAGAATTTTCTTCCTTGTTTAAGGCTGAATAGTATTCCCCTG
TGTGTGCACCACATTTTCTATATCAATTCTTCTATCAATGGACATTTAGATGATTTTCACGT

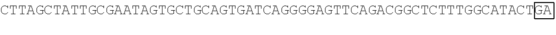

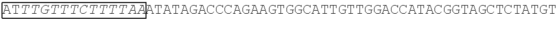

TTAGTTTTTTGAGGAACCTCCCCACTGTTCTCTATAGTGAGTGCACAATTTTTCAGCCTCCC
AAAGTGCTGGGATCACAGGCGTGAGCCACCGCGCCCGGCGCACTGTAGGATCTTTTTTAATG
CATTATATACCTTGCTGAGATTTTAGCAGAGATCACAATATTAAAAACTTGGGGAAGGATTT

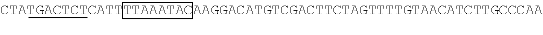

GAGCGGTGGTCGTTAATGTGTGGAGTTGGGATGACATCCAAGTCAGTTGGTTGCACGACCTT
TATTCTGTCTTGTCCCATAGATTTAGAAAGAGGCTGACACATCGGGTAACTAGTTTAAGGTC
ATCTGATCATGCGGGTAAGCGACATTTTTCAGAAACCAAGGCCCTCCCTCTCATCTCACTAG
TGGGAAGGGTGGAAAG
SEQ ID NO: 3
TTTTCCACTGCCCTTCTCAGCCCCACAGGCCTTCAGGAAGACCCGAACTTCAAGCAAAGCCT

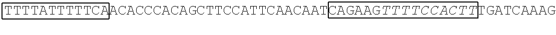

AGGCTGAACAAGAGGGCCAAGGGAGCAGCTGGTTCTCAGCAACTCTGGGCAGACCACAGAGC
CCTTGCTACCCACTCACTGTCCGTGCCCACCAGAGGACACAGCCTTCTCCCCAAATCAGCCA
GGTACATGCCCCAGAAAACACTGGCTTGCCTCGTTCCCACCTTAATTACCGGACCAAACGAA
CGTGAACACACTGTTTTCAAAACCAAACTCAATTGGGATCACGGGGGCCTCGGTTTCCTTGA
TTGTAAAATGGGTATATCGCCCCCCACTCTATTTAGCCCAAGAAAACAATTTATCTCTTGAT

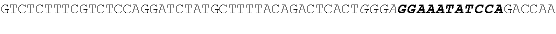

ATCCTAAAGCCTGATCTAATTTGGGAGATGCTCAGAAGTTTTGGTTCTATGCAAGAACAGCA
GTGGTAATAATCCAAGCTTGGCTTTAGACACAGGACGTTTCCTTAGGGGCATCTGGGATCTC

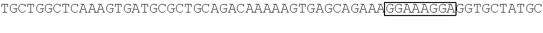

AGAATGAGCTTCTTCCACGGTGATACCAAATGGAGCTTTCAAAGGCCCACATCTGGAGGCAG
CAGCTATGCAGTGATTAACATTTTAAACGGTATTTTGAAATGGAGATCATTAGTAACCACAG
ATGTGATCTGACTCTGTCCCCCAGGTAATCTGTCTATTGTATCTAAATTCCAGACTTAGCCC
AGTAGACAGCTTGGGATGTTTAACAGGAATTGTCCAACACCATCCCCAAATCTATTTTTATT
CATGGAGTACTCTGACATCATCTCGCTTGGTCTTCCTGATGACTGTAATGCAGATTGGGAAC

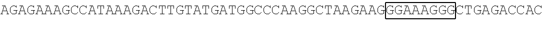

ACTTCAGGTTTTAGCTTTCAGGTGCAGTGAAGATTGAATGACTTAGCACGAGCTTTCAGCCA
GGCAGGCTGCAAAGTGCACCCAAGTTCTTTCAGTGATCGACACTTGCGACTTAGGTTGAGAT

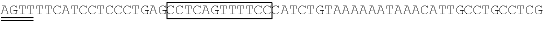

GAGGGTGGAGGTGGAAGTGGAATGAAGCCACATGTAACTCCTAGCGCTGTGCCTGAGACAGT
AGAGGTTCAATTATAGTAGTCACATACACACACAACACATACATACACAAGACACAACACAC
CACACACAACACATACATACACACCACACACAACACTTACATACGCACCACACTCGGCATGC
ATTGCAGATCACAAATGCACACCACACACACACTGCATACATATACCATAGAGAACACACAA
CACCTACACATAACCCCATACCACAAATACACACACACTACATAGTACACCACGCAGAACAC
ACACAGCCCCCATATTCCACACCACACCATCTCACTGCCAATTCCTTCCCCTCTTCATGAGT
TTTACGGCAGGTCCAGGTTCATCTGCCAGTTTAACAGATCCCAAAACTTCTGCAGGAGTGTT

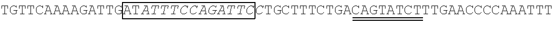

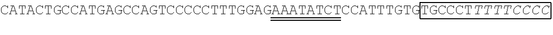

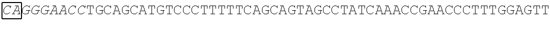

ATTACACTGCAGTCCGAGGGATCCGGCCTCCCTGAGACCCAGCAAGGACTCATTATCTGGGG
AGGTCTTCTGAGCCACAGGCCTCGCTGAAAGAAGGTGCAGCTTCTTGAACAGGAAGGCGTTT
TGTGGCAGAGTCTAAA
SEQ ID NO: 4
CAGGATAGCCCTTTCAATGCGGAAGTAGATGAAAGTTTACCCCTACCTCACACCATACACAG
AATGAATTCAAATGGATCATAGAGCTAAATATGAAGAGCAAAAGTAGCAAAACTTCTAGAAC
AAAACATAGATGATCTTCATCGATGTTGGATTAACAACAATCTCTTGAACAGGATGCAGAAA

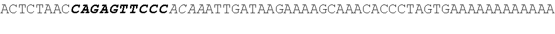

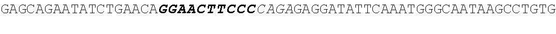

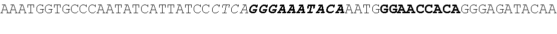

CTACACACCACCCCCACCACAGAGTGGCTAAAATTAGGGACTGATAATATTAAGTGTGCATG
AGGATATAGAGCTTTGTACAACTGCTTGGCAGTTCCTAATGGAGTTTCACGTGCTCTGTGAT
CCAACAACCCCACTCCTAGATGCACACCTAACAGAAATGCATGCACAAGGCACTCAAAGACA
CATACAAGAACGTCCTCAGCAACATTAGTTACAATAGCAAAACAACCGAAAACTTCCTGAAT
GCATATCAACAGAGAAATGGATAAATGAATTATGTTATATTTATTTTATTTATTTATTTTTG
AGCTGGAGTCTCACTCTGTCCCAGGCTGGAGTGCAGTGATGCAATCTTTGCTCACTGTAACC
TCCACCTCCTGGGTTCAAATGATTCTCCTGCTTCACCCTCCTGAGTAGCTGGGATTACAGGC
ATGCACCACCATGCCCAACTAATTTTTGTATTTTTTAGTAGAGACAGGGTTTCAACATGTTG
GCCAGGCTGGTCTCGAACTCCTGACCTCAGGTGATCCACCCCCCCCTTGGCCTCCCAAAGTC
CCAAAGTGCTGGGATTACAGGCGTGAGCCACCACGCCTGGCTGGTATATTTATTAATCAAAT
ACTACACAGCAATAACAGTGAACTAACTGTGGCTACAAGCAATAATGTGGATAAATTTTACA
GGCATAATTTGAGGAAAAAGAAATGAGATATGATAGAGAATATGCTATATGGTTCTATTTAC
ATAAGGCTCAACCACAGGCACAATTCATATACAAGTGATACAGCAGTTTACTTTCTGGGGGA
GGGAGGAGATAACTGGAGGAGGCTGAGCTGGGGGCAGAGGTGCATCTGAGGTTCTGAGAATG
TTCCAGAGCAGGCTATCTCCACCTTGGCACTAGTGAAATTTTGTTGGGGAGACTGTCCTGGG

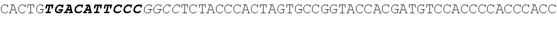

ACCACCCCCGCCTCATTTTGGAAACAAAAATGTCTCCAGACATTTCCAAATGTCCCCTTGGG
TGGAGGAGGTACAAAATTCCCCTGGTTGCCCTGTTTGATTTTGGGTCGTGGGGGAATTAGGG
GTGTGGGCTCATTTATGGAAATTCCTTGAGCTATACACATATGATTCATCAACTTTTCTGTA
CACATATAAAGCTTTTAAAGTTTTTAAAATAAAAAAACAAGTCAATGAATGACTGAAAGAAC
CAGTAAGTTTTCCAGCAATCCGAGACAATAGATAGAGTGGGGTTTAATGTCTTGTTCACAGA

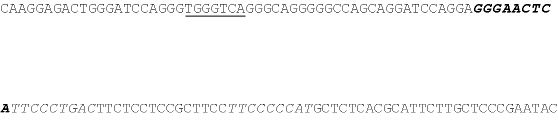

TAGCCAAGTCCCTACAGAGGCTGATCCCGTGCAGGGGCTCGGTTGGACCTTTAGCCTCAAAC
TCCCGTGGTGGAAACAGTTAGGATTGGTGGACGCTGCACCCAGCATCCTGCAAACAGAATTC
TGAAAACTGATATCTCATGCAAACAGGATATCAACTTTTGTCAGAAAGATCAAAAAGAGCTT
GAGGTTTTTCTTCACA
SEQ ID NO: 5
CATGTGCGTACAGCAATGTCTGGCACATAATCAGTGCGACTCTCATTATTGTAGTATGAATG

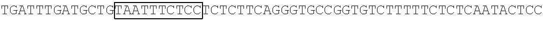

AAGAAATGAGCGTCCAGCATGGATGTGAAGGGATGCTTGCTGCACCTGGAGTTTCAAGATGC

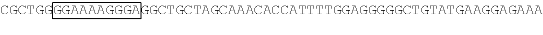

AGTAGCCTAGAGCCGGAACTCAGATTTTCTAAAGGGGATTCAGGCGGCCAGAGGATAGTAGA
GGGCCAGAAAGGAGGTCCCCAAGGAGGCTTCGGGGTAGGGTGTGAGGGGATCCTCAGGCAGA
CTGGGGAGCTGAGCCTGTTGTAACCGAGCGAGTTACAGAGAAACGCCGCACTTTGAGACGAA
TTCAGGGGTCCTTTATTAGCTGGCGACTGAGAGACAGCTAGTGCTCAAAATTCTCTCAGCCC
CAAAGAAGGGGCTTGATTTTCTTTTATACTTTGGTTTAGAAAGGACAGGTGGGGGTCTAAAA

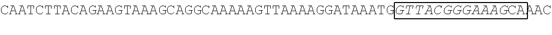

AGTTCCAGGTGCAGGGGCTTAAAATCTATCACAAGGTGATAGACACGGGGCTTTGGGCGTTA
TCAACCGGACACAAACGCCGGGGCTCTGGGTGCTATTAACCGGGCGAATTCCTGGGAACTGC
GGATATAGCTTGCCACAGTATCTTATCAGTTAATTGCATTCTTGGATGTGCTGGGAGTCAGC
TTGCACAAATTAAGTCCTTGAGGAAGCGGGGTGGGTAAGGGGCTGCAAATGAAAGAGCCAAG
ATGGAGTCTGTCTGGCTCTCTTAGCTAAGGGAGAGTCAATTCAGGTTAAAACAAGGTAGGGT
ATCACAAGCCCACTTAACAAGGGCAGCAGGACCCCAAGAAGAAAAGCTTTAGGAGTCTCCAC
AGTGGGCCCAGGGCAGTTCCACACAGGTCTCTGAGGCCCCACAGACAGGAGAGCTGTGACGA
CTCCCTTAGTGCCCAAGAAAGCAAGGAGGTGGTGGGCAAGGGGCTCCCAGAGGCCTTGGGGC
ACTAGAGGGGAGATGGAGCCGAGGGAGTGGCTCTGCAGGCCCCTCTCTGAGAGAGTGCATGA
GGTGTGGCCCCAGGCCCAAGGGTAGGGGGTGCACAGGGTGAGGGAAGGGAGGAGAGGAAGAG
GAGGAGGAGGTGGTGGCCACAGCGGGATGGCACTCAGCCAGGTTCAGCTTCGTGGGAAAGGT
CCCAGGTGGGCCGGGCTAGCACTGGGGATGCCCTGGCTCTGTGTCTCGGTTGGGTGGCTGAC
TCCTCCTGGAACGCATCCCTGACATCCTCCAGGCTGGCTTCCCCATACTCTGGGGACACACA
GTGCCCAGGCTTCCGGCCTCCCAGCCCTGGCTCCCTGTGGACTCAGCTGCCCCCAACAAGCC
TGCGAGCCACTGGAGAGCAAGGGCAGGCTGTCCTTGCCATCCAGATGCCTGGCACAGAAGGG
GTTCTCACCTTCGGGGTATGGGCTGACTCAGTGGGTTTCTCCTCCCTCCAGCTCTAACCTGC
CTGTGGAATAAACAAATCACTCCTCTGGGTTGTCTAGCTGGTGCAGGCCCCAGGGCGACGCC

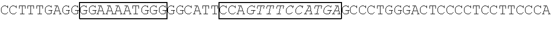

CCACTGCTGCCTACCCTCCGGTTCCAGGTATGCAGGCTTCCTCCCTTCTGACGGTTCCTGCT
GCTGGAGTCGTCCTTCCTGAAACCCTGCCTTTGCTTAGCCTCATTCCCATCTCTCAGTCCCA
TCCTGCAGCTGGGCAGGCAGTGCTGGGCCCCGGAAATGCCCTCTGCCTCCCTGGAGCACGTG

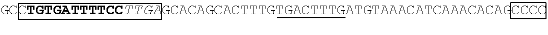

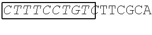

SEQ ID NO: 6
GCTTCACATCGCTAGTTCTCAGAAAGAGAGCGCGAGTTGATCTTTCAAATTTAGAGTCCGGG
CGAGTAGGGATGGCCAGTTTATGAATGTCTTAAAGAAGTTTATTAAAGGTGCGTGTGGGCAT

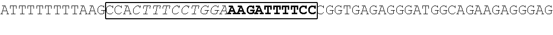

GCCCTACTAGACTTTGGACCACGTGGAGGTGGGGTGTTGGGGGTGGGCAGCCGGGTGCAGCC
TGCCCGGCCAGGGCGAGGAGGTGCAAGTCCGCGCCCCGCCCCCGGTCTCCATCCGCTCGGCC
TCGCGTCCGCCCACCGTGGAGCCCACAGTTTACAGAACTGCAAGCCAGGGCACATTTTATTG
TTATTATTTTTTAGGGTATGGGACTGGAAGGGATCTCTTTCTGTTCTCGCTCACGGACTCTG
GGCGCTGTTAAATTGTTGGTGCCATCGCCCCACCCAAGACCTTCGGGACGAAAACAAAAACC
CCCGTGTGCCCCCCGAGGAAAAGGCCCCGAGGGAGGCGGCGCTGAGGCGTCACGGCCTCGCC
ATGCGCCCGGGTGAAGCCGCCGGAGCCAGGCCTGGGGGCGGAGTGGGGTGGGTGCAGCTGGA
CTCGCCCGCGAAGCCTAGCCGGAGCCAGGCCCCGACCCGCCCTGGCCGCCTCCAGAGAAACC
CTGAGAAGGGAGAGGAGGCCAGCCGCGTGGCGCGCCCAGCCCTGGAGGCCAGGTCATCAAAT
TGACAAAGTTGGCCACAGCCTGGGGAATGGGGGGTGTCCACGGGGGCCGTGGTCTGGCCTTG
TCCCCGGGCCACGGAGAGGCCCCACAACTCCTCTTGTCCTCGCCGGTTGGAGATCAAACGGC
TGACAAGGCAGCTGCGTCCTTCCCAGTCTGACAGGCATTTCCAAGTCCTACCCTGGGCCGAG
GAGCGCGGGGGGCGGGGTGTGACTTCCAGGTTGCTGTGATTGTCAGGGGCGCCTCCTGGAGT
CACCCACCCGAGTATCTGCGGAGATTTGGCCACACCTACTCCTGGGCTGGCAAGTCCAAGTC
CCTCTCCCACGTGGAGCGCCCCGGTCCCTTTACCCTCTCGCTTGCCATAGACCCAGTTTAGG
AGGTGGGGTCGTGTTTGACCCCAGGAGTTAATCGGGGTAAGGTGAGGGGAGGGGACAAAGAT

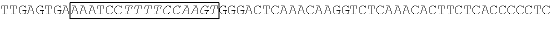

ACGTGCTCTTGATTCTGTTGTCTTAGTGTCGTTTATTTTGTCATCCTCACCCTCCCAGAGTC
TGTAATACCAGGATTTATATGGAGACACTTTTTGGAAGTTGAACTCCAACATCTACTCCCCT

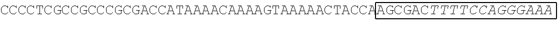

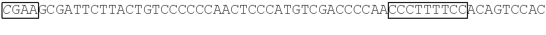

GCCCCCACCCCCAAAGTCCAAGTCCGAAAGAGCTTTTGGGTGGGTGGAGACTTGCATTGATT
CAATTTAGTTCACTCACACAGCACCCCGCCCCCCTTCTCAGGGGGTCCTGCTCCCAGGATGG
AGGAGATATAAGGACGATTTTTCTTTTATTTTAAAATAAGCTGCCCAGTGGCCCCCCCCAAC
CCCCTCCCGCTGTTGCGCAGCCGGGGCTCGGGGGAGATGAGCGCACAAAAACGCGGTTTGCA
CGTGTGTCCGGCTTGGGCTGCGGGTGTGCGCAACTGGCGACTGTGTGTGTGTGTGTGTGAGT
GTGCGCGCGCGCGCGAGTGTGTCTCTGTGTGTGCTTTCTTGTTCTCTTACAGGGTACAATGT
TAAAAAGCCACCGCTAGTCGCCCCCAGTGCTCCGACTCTCTGGGTCTTTTTGTCTCTAGTGC
AGATTAAACGTCACGTCCGCACTTGAACTTGAATTTTATCCCATTGTACAGAGGCAGCCCCA
GCCATAGAGAGACCGA
SEQ ID NO: 7
TAGAAAGCTTTCCAAATCCTGGTGCCTGGCGTATTCCAATAGTCTTCTTCCCAGTCTTCCTG

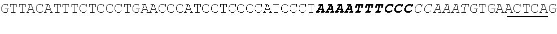

TCATACCAGTTTGCTCCTTATGTTTCATTGGCCCTTGCTGCTAAGAGCATCCGCTTGCACCT

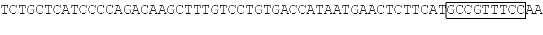

CTTTAGCCCATGTTATTCTTCTTGTCTGAATATCCACCCTTTTCTCTGTTCTCAATAATAAG
TTCAGGCTTTTCGTCTTCTGAGAAGCCCTTTCTGACTTCCACAGGCTGAACCACTGGCTTCT
GCTCCTCTACATAATACTTCAATTCCAGCATTGATCTCACTCTATCATGATCATGGGTTTAG
CTGTCTGTCCCTGCCACTGCTGTGTGTTCCTCTTGAGGGCAGGAACATTTGTTTTTCACTTT
TTAAAAAACCTCTGTTGCCCAGTCTGGCATTAGGAAGTGCCCATTAGGTTGTTATTGCTTGT
TGGCGCTTGAGCTGGGGCTTGAAGGTTTCTATAATGTGTAGCAGTGTATAGAAAACAGGCAG
GTCAGAAAAGGCTTCTGTGCATCACACCAACATGGCACATGTATACATATGTAACAAATCTG
CATGTTGTGCACATGTACCCTAAAACTTAAAGTATAATAATAATAAAATTTTAAAAAAAAAA
AGAAGAGGCTTCCTGGAGGAGATGACAGCTGAGCTAAGTCCTGGAGGATGAGAAGGAGTATA
AAATAAGATAATAGGAGAAAAAAGGCAGTAGGAACAGCATGGGTAAAGGTGATGAGGCCTGA

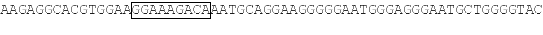

AGGCCAAAGAGGGAGGCATTTGGTGAGTATTCTGCAGAGTCTCCTCTGCTGTGCTGAGGTGT
GGACAATGGGAAACCATGGACGGACTGGAGTAGGCAAATGTCATATTCCCTGTTACAACTGT
CTGTTTGCATGTCAGCCTTCTAGAAGCCCCTTAAGGTATCAACTATGTTTTTGTTTTGTCAT
CATTCAATCCTAAGTGCACAGAATTCCGGGCATATTACAGGTTCCCCATGAATGTTTCTTTC
TTTATTAAAATGTATGAAAACTCTCCAGATTTAAGGAAGGTCCTCAATGTTTCAAATTCTTT
TTGTTAGATCATTGGTCCTGTCTACAGCTGTCACAAATTTAAGGACTCTGGTTATATTTAAT
CTTCACTTTTGAATTTTCTGCTTGAAAAATTTGTATTAGAAAAAAAAGTCTATCCTTTTATG
GACGGCTCTAATCTCTTGAATCATTTGGGTTGGCTTTTCTTTGGACCTTCTTCAACTCTGTT

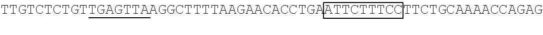

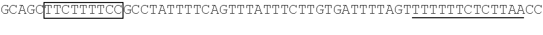

AAATGCTAAATGGATTTAGGAGAAATAAACTTATTTGTAAAGCTGTCAAGGGACCATTAGAA
GGATGGTGCTTCACAGATAGAATACAGTTTTTATTAATGATGCCTAGACAAATCCTGCCATT
AGCCCAAGGGCTCAGAAAGTTAGCAGCCTAGTAGTTTTGGAGTTGTCAATGAAATGAATTGG
ACTGGATGGTTAAGGATGCCCAGAAGATTGAATAAAATTGGGATTTAGGAGGACCCTTGTAC

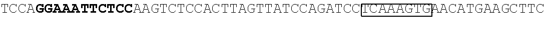

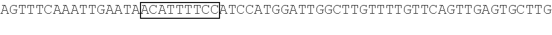

AGGTTGTCTTTTCGACGTAACAGCTAAACCCACGGCTTCCTTTCTCGTAAAACCAAAACAAA
AAGGCTTTCTATTCAA
SEQ ID NO: 8
TGAGGCGTGAGCGTCTACAGTGAACCCAGCACAGAAACCTGCTAGGGGAGCTGCTGTTGACT
GCATCGCGATCCAAAGGACCGGCGTCTTTTGTAGATGCAGGGGCTGAGCCAGGCCAAGCGCG

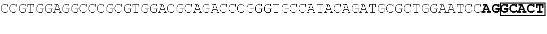

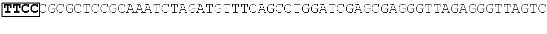

AGGCCAGGGTCAACTTCAGAGTCATGGGCTCCCTAAATGCGACTTCTAGGGTTGAGTTGCTG
TGGACGAGCGACCCATGTCGGAATCCCGCGCCCACGTGGCTGCCCAAAGTTCCGAGTCTCCG
GGCTGCAGGTTCTAGTCACGGAACCGAGTTGGGAGAGTCATAGGGGCTGGGACTTGGAGGAT
CGGCTGAGGTCCGGTGCTCTTGGCTGTGTTCGCGGCTCGGAGCCGTCGCCTGACTGAGGGGC
CCGTCACAGATGTGTGATGTATAAGCTCTGCACGCAACAGGAGCTCAATAAATGTGCGAAGG
GGGGTATACTTATGTTCGCACTGTATGCAGGCGGCCTAGAAGGAAGTCCCTGATTGGCACAG
GGATGGAGGATGGGGCAAGAGCCGCAACAGCGCCGCGGAGTTCCAACGCTGCCGGTTCCCTG
GGGTACGAGCACAGCCTCAAGCAGCCTCAAGCCCTAGGAAGCCCCCAGTTCAAAGCACAGGG
CGCATTGGAGCCTGGGCACGATACAGTTCACACCACGGCTGCGATGGTAAGCCACGCCCAAG
TCCCAAGGGCCTAGGGGACCCCCGCCCTCCACAGCCGGAGGAGAAACCTGGGCGCAGAAAGC
AGGGGGAATATCTGGTTGTAGGTGAGTAAGCGGGGTCAGGAGTTCCCGTTAGAGTCTCTGCG
TTTCGGGAGAAGGGTGATCATTCCCAGGCTTGTCCGACGTCTCTCTCAGGGTGCGCTCCGGA
AGAGCGAGCCCTTTAAGGCTATGCCGAGTGGGCGCGTCCCGGCCTCTCCCGGGAGAGGAGAG
GCGGGGCGGACCTGTGTCCCGCCCCCGGCCCGGCCCGCCCCCAGTGCCCGCCCCGCCCCCGG
CACTCGGCCGGCGGCGCCTTTGATGTTCCGACCCGCCAGCTCGCGGAGCCGCTCTGCCCCGC
GCCCTAGCCCGCGCCTGCAGCCCGCCCAGGCGGAGTCAGCCCGCGCTCCGCCCGCCGCGATC
CGAGCTCGGAGGTTCGGACTCCGGGCTCGCCGCCCCCCGGGCCGGCTCCGCGCCCCGCACTC
CCGGCGCCCAGCGCCCCGCGCCCCGGCGGGCGGAGCGCACCATGCCGCAGCTGGACTCCGGC
GGGGGCGGCGCGGGCGGCGGCGACGACCTCGGCGCGCCGGACGAGCTGCTGGCCTTCCAGGA
TGAAGGCGAGGAGCAGGACGACAAGAGCCGCGACAGCGCCGCCGGTCCCGAGCGCGACCTGG
CCGAGCTCAAGTCGTCGCTCGTGAACGAGTCCGAGGGCGCGGCCGGCGGCGCAGGGATCCCG
GGGGTCCCGGGGGCCGGCGCCGGGGCCCGCGGCGAGGCCGAGGTGAGCCCCCGCCGGCGCCG
GCTCCTCCCCCGCGGTCGCCGCGCCGCGCCGCCCCAGTTGCGCGCGGCCCTCGGGGTCTCCA
GCGCGCAGAGCGTCCCTGCCCCGGCGTCGGCCCCGACCCCCGCGGTCCCACCGCCCCTCACT
CCCCTCCGGTTCTCCCTCCAGGCTCTCGGGCGGGAACACGCTGCGCAGAGACTCTTCCCGGA
CAAACTTCCAGAGCCCCTGGAGGACGGTGAGTTTCTGCCCGGCCCGGCTTCCCTTCGTCGCG
CTCAGGCCCTGGCCTCGGTGGGACGGGGACGCCAAGGACCGCGGGGAGCCGGGTGCCTCCCC
CACCGCAGCTCAGGAGGCGGCAGAACCCAGGGGTGGAGAGTGGGGGGCGGGCTTCCCGGGCG
CCGCCGGGTCGAGTCA
SEQ ID NO: 9
GACCAATATGGTGAAACCCCATCTCTACTAAACACAAAAAATTAGCTGGGCGTGGTGGTGCA
TGCCTGTACTTGGGAGGCTGAGGCAGGAGAATCGCTTGAACCCGGGAGGTGGGAGTTGCAGT
GAGCCAAGACTGTGCCATTGTATTCCAGCCTGGGCAACAAGAGAGAAACTCCATCAAATAAA
TAAATAAATAAATAGGTCATGGGGATTGATTTCATCAGATTGCTTATCAGAATCAAAGGAAA
TGATTATAGGCTTTGAAAAATATAGACCTTATATTTATTATTAGTGGATTTTGCAATATTGA
ATCATTCTTGCTACCCTGGAATAAACCCTACCTGGTGATTATGTATTTTGTTTAATATATTA

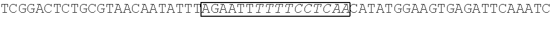

TGGTGTTCTTTAATTGTGTGCAATCTTAATCACTTTAATATCAGTTTTGAATTCATATCATA
AAACTAATGAAGAAGGCTTTATGTTCTATGTCTGTATATTTTAAGTAACATTGAAATTATCT
GCTCTTCAGAAGTTTGTTGTAGTCTCTTTGTGAAATCTTCTAGGTATGGCATTCTTTTTGCT
GGGGACAGGAGGAGCTCTTGATGCTTGAGGGAGTCTTGGAATGATATGTTCACGTGTACTTC
TATCCCAGTCCTAGTTGTCTTGGATGACTGAGGGCTCTAGGTAAGAGGAAGAAGGAAAGAAG

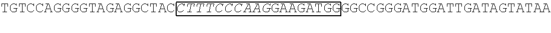

AGGACTGAGAGTAACAATGATTCTTTGCTTGACCAAACTTTGTCAGCAAAGTTTGTCCTGAA
CCTTCTCCTAGGCCCATCCATGCACTTCCTTGTAAAATCCATTTTTTAGCCAAGAACAAGGA
TAAATCAGTTTTGCAAGAACCCCTATCCCCCTGTATCTGATCACCCTTGATATCTTATCAGG
TTTCTCATCCTCCACCATTTCTCAGTTCATGTCTGATCTACCCTGGCCTGTCTTTAGCAAGA

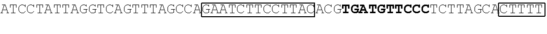

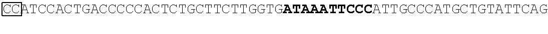

AATTGAGCCCAATCTCTCCCCCTCTGCAAAATCCCATTGCCTTGGTCTCTATACCTATTGTG
ATGGTCCTGAATAAAGGCTGCCTTACCTTTAAGAAGTGTCATTGCCTAAGTTGTCTAATTAT
ACGATAGTTTAATCCCATCTAGAATAGAGGAGGAGAAGGAGGGAAGCAATGTGGGTGTGTAG
GAGGGTTGTGAAGAGAGGCGGCAGGAGAGGCCTAAGCTGAGAGGATCAGCCGCCTCTTCCCA

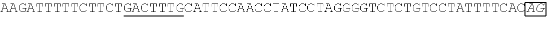

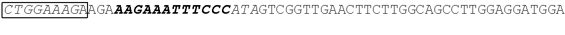

GGAGATAGATCCTCAGAGCCACCTCACGGGAGGGAGTTTTGACAAACAGAAGTCAGCGGTCC
CACTCCCAAGTTCTCCTCATTTGTATTTGCCAGTGTCAGCCAAGTCCCATGGCTTGTTAATA
AGCCTCAGATCACGGGCAGTCCTTTCGCGTGGAGTCTGATTTTCTGAGGCAGCTCTCTCCAC

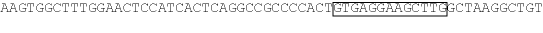

GAGACAGCTGGGGGTGATGGGACACAGGGCGGGCCCTTCCTGGTGCCTGGGGCGGCCAGGTC
CCCTTGTCCCCCCTGTGTCAGGGCTCCCGTGCTTCCTCGCCCTTCTCTCCTAACATCTAAGG
CAGGAGGCAGGGCCTTCTCGCCCCCCGCCATAATATAACCTGCCACCAGGAGGCGCTGTTCT
ACAGGATGGAGAGTGG
SEQ ID NO: 10
TGGTCACTATTTACTGTGTGAGTTCCACTGGAGACAAGCACACTTGCTCCAATCCCAGAGTC
TACACCTGACCGGGAATTTTTCATTTTGGATTCTGATTGAGGGAAAATACTGATATCCAATG
GGCTATAAGGGGGACCTTTCAGTTTCTGTTAACAGCATCTGAGTTCTATGCACGTCCTGGAA
AGGCACCTGCCCACTGGCTAATTCACATGTTGTAATCCCAACACTGTAAATATCTGACTTCA
CATTATACCCATGTAAATCCTGTCTCAGTAGTTCTGGACTCAGCCATGGCTGCACTAATGTG

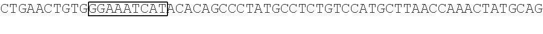

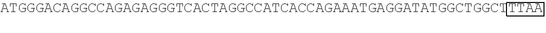

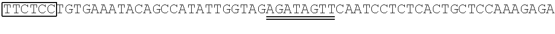

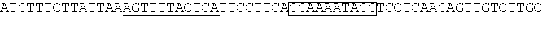

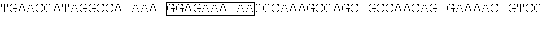

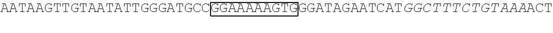

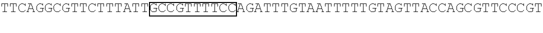

AGGAGTATGCCGTGCTAGATGGACAGAAGTCAACTTGTCAAATCCTCTTCCTATTTCTACTT
GGAGCTCGTAGTGAGAAACGTTGGCGGAACATACTACTTCACTGGCTCTAGTGGATGGAGGC

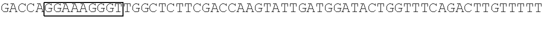

CATGTTTGAGTGATTCAACTTGTGTTCTTGAACTAAAGCTCACAGAAAGTGACAAAAATCCA
AATATTCCAAAGAAGGCTTTTGTTTTATCTTCCTTTGAAGATGCAGATATATCCTACTTAGC
GGCGACGACGGCCAGCGACTCCCTACAGGCTGCCAGTGGTGCGGACCTGGGCGGGTGCCGGA
GCCCGGATCACAGAGGGGCAAGGCGAGCGCTCTAGGCCCACTCCTGGCGTGGGGCGCTGGAG
GGTGGCAAGGCCGCGACAGGGGCTGTGGGCTTGGAACTCTAAACATAGTATTCTTACGTAGC
CAACTGGAATATCTGGTCAACCGTGAGAGCTTTGGGAACCTGCCTCTCACCTCAAGAAACCT
CCAGAGCAGATTGGAACCATCCACTGGGTAAATTCTGGAAAATCTGTCTCTGCCTGAGACTG
GGGTAGGAAGATGTGGGGTAGAGAGGAAGGTGGGGAAGGGATGTAGGATCTATTCCTTGGGT

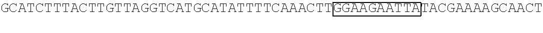

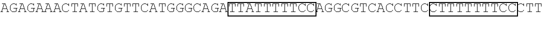

GGTTCCTAATGATTTAGACTCTGCCTTTGAGCCTCAGTGCTGTTAACTTTTGAATGAGTAAC
TTGAGGCCTTCCTGAAAGACTATAATAAAAAACCCATATTATACAATCACTAAGAACCCCCA

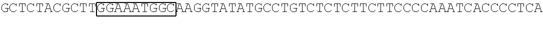

GCCTCCCACTCCACCCTGGGAAGACAATCAGGTCCTGTGCCTTGTTTTCTAGGCAATATTTG

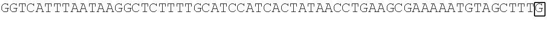

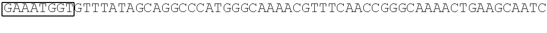

ACCAGATTATGGAGCCGTATTTCTGTTTCTTTAAAGACAAACATTTTTGTGTGTGTGGCTCC
ACCCAGCCTGAGCTTC
SEQ ID NO: 11

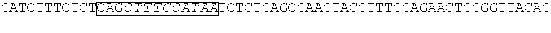

GGGAAAAAGCCAGGTGTTAATGATGAAAAAACATTCAACTTTTCTACCGCTACTAATAACAT
TTAATTCAAGTACTGAGAACATTTACCTCAAATCTTCAAGAATAAGACAATATTATCCCCTT
TCTCTTTTATTGTCGGACTAGAGAATGTGAGAGAGGTTACATTCCATGGGCTTTGGGAATTT
AATATGGTTCAAGGATAAACACACCCAGGTTTTTTCACTCCAGAGAAGAGCTTCAAATATAAT
CCAGTTTTCAGGTCATCAGCTCAGCTCTTGTATCCCTAACAATGCGGTTGACATACCGTCTT
CTCACATAGTCTAAACTCCTAAACTCACTAAGCCATACTTTAAAGTACATATAAAGGACTAG
AAGCACCAAGCTACCAGTGAGACGAAGAGGAGAGTTTCCACAGAAGCTGGCTTCAAATAAGA
CAATGAGTTCATCTTTAAATACTTGCCATTTGAGGTGCAGATGGATATAGTTGGCAGGCTCC
TATCTAACGCATGTTATGCACAAGCTACCGTGAATTGATAATATCAAAACAAATATCCAGCG
AGCCTCTGCAAGTGTGCATCTCTATTTCACACCAATTATAGTTGACTTAATTCCTCCCTCAT
TCATCTCCCAGAGATGCAGCCTCCTCTTAAAGAAGTTGCGGCTGGTGGCCCATTCAGTGATG

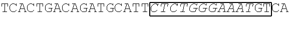

SEQ ID NO: 12
TAGCTCCAAATAACGTGAGATTAGGTCTCACATTAGGGAGATTTCTCATCCCTACTTTTGCC
TTAGATATCTTGCAACTCAGGTAAAGGATAATGTGGAGGATCCTACGAATAGTAATCAGGAC

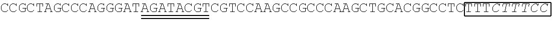

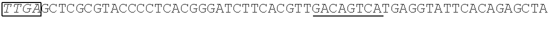

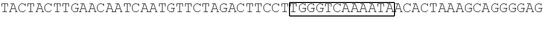

GAAGGCATGGACAATGTAGTGATCAAAACAGCCGTGCCAAACACTAACTATACTGATGACAC
AAAACGCAGGAAGCGCAGGGCAGAACGCGCTCAGTACCAGCTTTCTGGCGATCTGCTCCTGG
CCACTCTGTCCCAGGACCTACAAGGCCACCGCCCCAGCCCACTAGTCTCAACTTGCCCCCTC
CCCTGTGGCTGTGCCTTGACCCTGAAAATCTTCGCGCACTCCTGGGTTTCACCACCACACAC
ACGCGAGCGCGCGAGCGCGCGAACACACACATCAAAAAAGAAAAAAAAAACCGTGATAAATG
CCCTGGAGAGTCTAAGTGCATTAAGGACCTCGTTCTCGGAGACCCCAGACATGCCGATGTTG

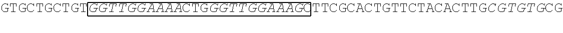

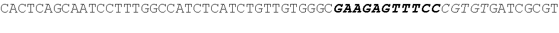

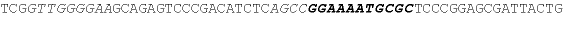

GCGGCGTCTGTAATTGCTTATTAACAGCGAATATTCAGGCTTCTCCTTATCCGCAACGAAAC
GTGCCCCCCGCTTCCGTAATAATGAAACGATAAAATATGACGGCCCCGCTCTTGAATCTATC

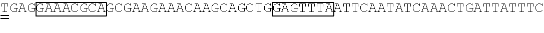

ACTAATTATTCTACCTTCTGTATTTGCCGCAGAGCAGAGGCGCAGGGAATCCTAACTGTGGG

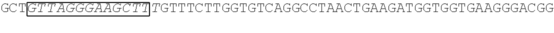

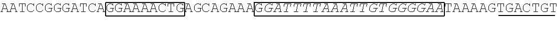

CTCCCCGAAAGCAGTTGTGTTGGCGTGAGTATGAAGTTGGTGTTAGCTCGCTTAGTTTCACT
CTTGTCCTGTCCCCAGTACCCTGTCCAAGCTTCTGATTTAGAAGCTGAGAGCTTAGGTCCTC
CAGGATGGCAATGTGCTCTGGCCGTCCCCGGAATCCCAAAAGCTTAGCGCGAGTTTCCTCTC

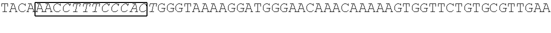

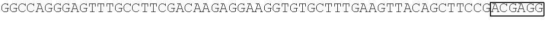

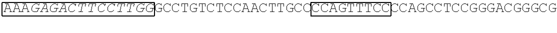

CTTCCCTGCAAGCTATCAGCTTGAAGAGTCTTTCTCTCTCTGGCCTCTAAAATCTGCTCCAG
CCATCTCATTCCTCCAGCGGAGGGCTGAGATGACTAATGCGCCAAGGGTGGACACCGCTATG

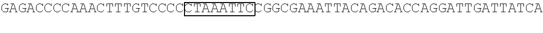

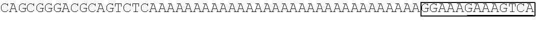

CAGGCGACTTGATCCAATTAGTAAATGCCTAATTGAATTAGTGTCACCTCCACCAGCCAATA
GGAGGGTCTCCGGGCTGTGAGGCCGGGAAGCCCCTCCCTGGCCGCAAATATATAAAGCAGCT
AGTGACAGCCCATAAG
SEQ ID NO: 13
ATAATTTCTAGCCAAATAAATGAGGATAAGTGTGTGAGTGTGGTGGGGTAGTTAAGAGTTTG
GAATCCAGAGTTTCACTGTCTGGATTTGAACAGGCTCCGTTGCTCATCAGCTCTGTGACTCC
GGGCAAATGACTGAATCTCTCTAACTCTCAGTTTTCTCATCTATGAAATGAGAATAAGTAAC
AGACTCTCCCTCGCTGAGTTGTTTGAGAACGGGATCAGACAATGCACGTGGAGCCCCGTGTC
TGGCACACACTATGTCCTCATTGCATATTTATTATTAGGTTTCTGCTTTGTATTTTTCTCTG

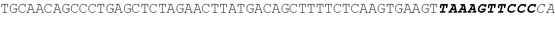

TACTCAGTTCCAGAGCTAGGCCTCACCTAGGTTGGAAGAAGTCAATTGTTCTCCACTGCCTG

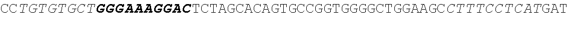

GCTACGGGGAGTACAGTGAAAAGGGAGGAGATTGGGGTAGCAAGGTCCCAGGGGTCCTGGCA

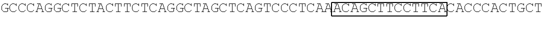

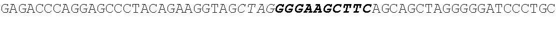

AGGATCAGCCTCCTCCTTACCTTGCAGACCCCTTGGCTGTGGTCTGGCATTCCTAGTGGCTG

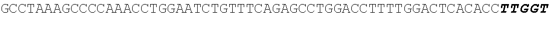

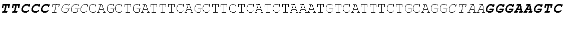

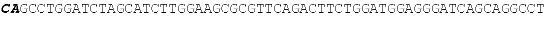

TGGTATTGGTGGTTCTACCTGCTTCGGTTAAGAGTGAGGACTCTGGAGCCAGACTCTCTAAA
TCATAGCACTGACACTTACTATATGACTTGGGCCAGGTTCGTTACCTGTCTGTGCCTCAGTT

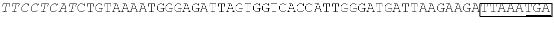

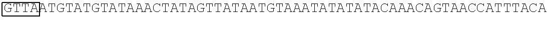

TGCATATAAAACATAATATAAATGTAATATTAGCTATATTCATATATAAAAATAGGTATATT
TTATATATAAAATATATTACATTTAATTTTTTACATTTTGCATTAAGATATATTATTTCATA
TAAAACATTTAAGTACATATACATATAATTATCTATGTAATTATTATTGTTATTGTTGTTAT
TGTCTACTTTACCTCCCTGCAGGGATGGGGAGACCACGGCAGGCTTTTAAACATTCTTGGTC
TCACTGAGCCGTAGGTGAGGAATCCCTGGTTCCTACAGACTCTGCTGTAATATGAGGAGCAG

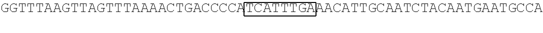

CATAAATATCATTTCTCAGATTCCTATGATGCTCTTCTTTCAGATCTTTTCACTTCAATTTC

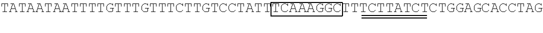

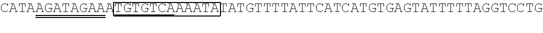

TTAACCCCCATAACTATTGATTCAGAGAAGTAGGGTGGTTCTGAAAAATACAGGCATAATCT

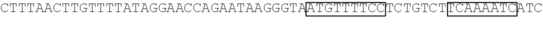

AATAATCCATGCATTGTTTAACTCATGTCATAAGCAATAATGCCTTTCATATAGCCATTGGC

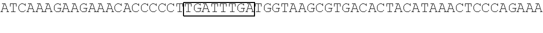

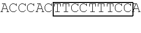

SEQ ID NO: 14
AAAAATACAAAAGTTAGCCAAGCGTGATGGTGCATGCCTGTAATCCCAACTACTCAGGAGGC

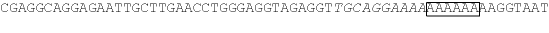

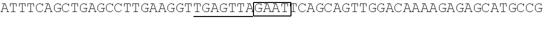

ACTGGGTTCGGTGGTGCATGCCTATAATCCTAGCATTTGGGGAGGCCAAGGCAGGAGGATTG
CTTGAGCCCAGGAGTTCGAGATCAGACTGGGCAACATAGTGAGACCTCATCTCTACAAAAAA
ATAATCAGCTGGGCATGATGGTGTGCACCTTTAGTCTCAGCTACTGGGGAGGTTGAGGTGGG
AAGATTGCTTGAGCCCAGGAGGTCTAAGGGGATTGCGCCACTGCACTTCAGCCTGGGAGAGG

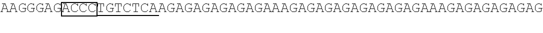

AGAGAAAGAGAGAGAGAGAGAGAGAGAGAGAGCGAGAGAGCGCACGCATTTCAAAGCAGGGA

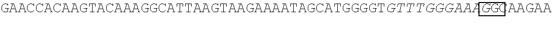

TAGTTCAGAACACAGGCTGTGTGTGTGGGGGTGTGTGGGGGTGCCTGTGTGTGGGTGTTTGT
GTGTGTGTGAGTGTGGGTGTGTGGGGGTATCTATGGAGGGGATGCCTGTGTGTGTGTGTAAG
GTGAGTGTGTGGATGTGTGCATGGGTGTGGGTGAGTGTGTGGAGGTGCCTGTGTGTGTGTAT
GAATGTGTGTTGGGGAGGGGAGAATGTGTGTGTGTGGGGTGAGTGCGTGTGTGTGGGGGTGA
ATGCGTGTGTGTGTGTATGTGGGTGTGACTGTGTGGGTGTGTGGACGAGTGTGTGTGTTCCT
GGGTGTGGGAGCCTGTGTGTGTAGGGGGAGTATGTATAGGGTGTGTGCATGAATGTGTGTGT
GGGTACGTGTGTGAGAGTGGGTGCCTGTGTGTGGGGGGTGAGTGTGTGTGTGGGGGGGCACT
TGTGGAGGGTGAGTGTATGTGTTTACTGAGTGTGAGTGTGGGTGCCTGTGTGTGGGAGGGTG
AGTCTGTGTGTGAGTGTGTGGGGGAGTACCTGTGAGGGGTGAGTGTGTGTGTTTATGTGAAA
GTGTGTGTGTGTGGATGCCTCTGTGGAGGTGGGATAGGGGGTGCCTCTGTGTGTGTGTGTGA
GAGTGTGTGTGTGTAGGGTGTGTATATGTATAGGGTGTGTGTGAGTGTGTGTGTGTGAGAGA
GTGTGTGTGTGGCAGAATAGACTGCGGAGGTGGATTTCATCTTGATATGAAAGGTCTGGAAT

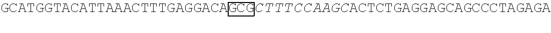

AGGAGGAGCTGCAGGGACTCCGGGGGCTTCAAAGTGAGGGCCCCACTCTGCTTCAGGCAAAA

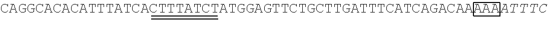

CACTGCTAAAACAGGCAAATAAACAAAAAAAAAGTTATGGCCAACAGAGTCACTGGAGGGTT
TTCTGCTGGGGAGAAGCAAGCCCGTGTTTGAAGGAACCCTGTGAGATGACTGTGGGCTGTGT
GAGGGGAACAGCGGGGGCTTGATGGTGGACTTCGGGAGCAGAAGCCTCTTTCTCAGCCTCCT

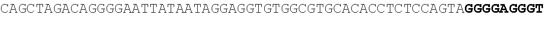

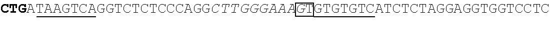

CCAACACAGGGTACTGGCAGAGGGAGAGGGAGGGGGCAGAGGCAGGAAGTGGGTAACTAGAC
TAACAAAGGTGCCTGTGGCGGTTTGCCCATCCCAGGTGGGAGGGTGGGGCTAGGGCTCAGGG
GCCGTGTGTGAATTTA
SEQ ID NO: 15

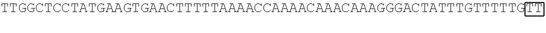

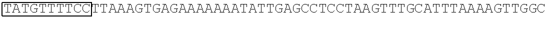

AAAGATGAGTCTCAGTTAGTGGGTTTGATGGCTCTATGAGAAAGAGAATAAAATTACTTACG
ATGACACTCCATGTAGACTGAGCAAACGAAAGACTCCTAAGTATTGCAACAGCTACTGATGA

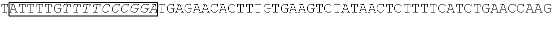

CAGGTGTAAAATGTACTTTCCTCAGAGGAGTTTGTAGTCTAGTTTGAGAAGATAGCACTGTA
TTATCTAGGTTTGCTGGTAGTGACAGAAACCTCAAAATAACTGAGACTTAGAAAATAGATAT
TAAAAAATGTAATCCAGGGCTGGCATGGAAGATCCATGGTCACCAGGGTCCCAGGCTCTTTC

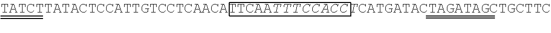

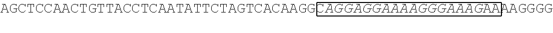

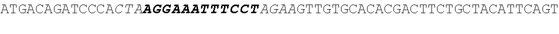

TGACCAGAAGACAGTCATACAACTACACCTAGCTACAAGGGAGGCTAAAATTTGTAGTCTTT
ATTCTTGCTGGCCAGATGCCCAGATAACTGTCAGGGATTCTGTTACTCTAGTAAGAAGGAGA
GAGTAGCTATTTGGGAATAGTAGCAATCCCTACCTCAAGCCCATACTCAGAAAAAAAAAATA

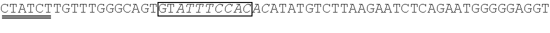

AATTTAAAATATACCAGTACCGTACCCTCCTGGGTGTCTTGATTATGCTGGGGCATCCAGTA
TATGAAATAAAATTAGGCTGTTGTCTGGTAATTTACATTTAAAAATAAACATTGGAGAATTT

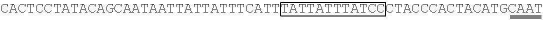

ATCTGCTTACACTAAAATCTCTTGATTATTAACATAGTAGAGGTAACTGTCACACATTATAC
TTCAACTATGTACTAACTCTAAGGTGACATGTATGACTAGCACCTACCATTCATAAGATGTA
TGGGATTCAGTCCTCCAAAGGACCCTCACAAGGAAGCTAAGGATCAGAGAAGTCATGGCTTG
CTCAAAGAGAAACAGCATAGTAAGTGACAGAGCTGGGAATCCAACACAAGTTTGTCTGTTTT

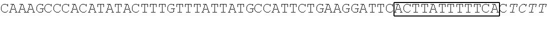

CCACAAAACGTCAGGACAGAAAGGCAGTCATCTATATGTAGCATTGAGAAATTTCTATGGGC
TTTAATTTAAATCTTAAAAACTAATAAACTTCAGCAACATATTTCTTCACTTTAATGTGTGA

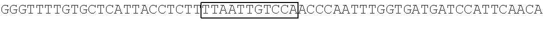

AATTTTTCTTGAACATCTGTTTTATGTGTCAGGTACTGTGATGGTTGCTGAGTATATCATGG
TAAGCAAAACCACACATTGTCCTTACCCCTTGTGCTACATGATTTAACTGCTGGATGGATTA
AACCAGGAACTATAAAGATTAAACTGTAATCCTTACCACAATTTTGAGTTTACAGTTTTTCA
AATATCATTTCAAGCCATTAGCAAAGGCTGTATGTATTTTTTACATATGCCTCCTCGTTTTG
TGAATTTTGAAAGGATGTGGTTTCGGCCTTTGACATCAGAGGAGAAGCTCAGCTATGTTGGC
TGAACGTTGATAGAAAGATAACGTTGAAGGCAAGTTGCCCTTGAGCAGCTCTCTGAAGATCA
ACTGCCTCCACATTGC
SEQ ID NO: 16

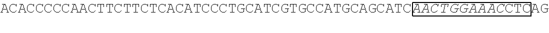

CATCAGCAAACGACGACAGAGCGTTCATCCGTAAGGTGAACCAGAAAAGCCAGTTCAATGAC
TTGTTTAACCATGGTCCATCTCAGAACCAAGAGTTGGGCCTCTTATTTACCAGAAAAATTGT
GGGGGCTTTGTGATATGGCTTTAAAAAAATCTTGTAATTGCCAGGCGTGGTGGCTCACACCT
GTAATCCCAGCACTTTGGGAGGCCGAGGTGGGTGAATCGCCTAAGGTCAGGAGTTCGAGACC
AGCCTGACCAACATGGTGAAACTCCGTCTCTACTAAAAATACAAAAACTAGCTGGATGTGGT
GACGCGTGCCTGTAATCCTAGCTACTCAGGAGGCTGACGCAGGAGAATCACTTGAACCTGGG
AGGCAGAGGTTGCAGTGAGCCAAGATTGTGCCATTGCGCTCCAAAAAAAAAAAAAAAAAGAC

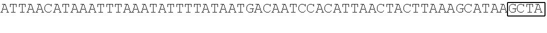

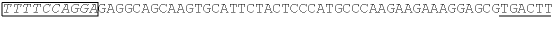

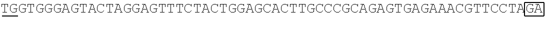

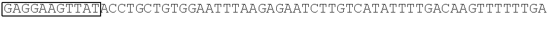

GATGGAAGTCTCACTCTGTCGCCCAGGCTGGAGTGCAGTGGCGCAATCTCAGCTCACTGCAG
CCTGCACCTCCTCGGTTCCAGCTATTCTCTTGTCTCAGCCTCCTGAGTAACTGGGATTACAG
GCGCCCGCCACTACGCCTGGCTAATTTTTGTATTTTTAGTAGAAATGGGGTTTTACCATGTT
GGCCAGACTGGTCTCAAACTCCCGACCTCAGGTGATCTGCCTGCCTCAGCCTCCCAAAGTGC
TGGAATTACAGGCGTGTGCCACTGCGCCTGGCTAATTTTTTTTTTTTTTTTTTTTTTTTTTT
TTTTTTAGTAGAGACGGTGGTTTCACCATGTCATCCAGGCTGGTCTCAAACTCCTGACCTCA
GGTGATCCACCCACCTTGGTCTACCAAAGTGCTCGGATTACAGGCATGAGCCACCAGGCCCA
GTCAACGTGATGTGTTTTGGAACCCTGAATTCCTTGGCTTGCCCGGAGGGTTTTCTTTTTGT
TAATATCTTTGCTTGCTTTCTAGTATTTAAAAAATTGTGTTTTGCTCTAACTATGCAATGGC

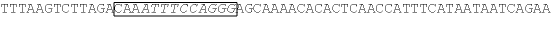

GAGAGCTCTGATCAATAAATAAGCAAGACTGAATTTTACAAAATAATCCAAAGTTTAAAACC
AAAGCCCACTTTTTGCATGATCCTTTAAGAGAAAGAAATCTGGAAGCAAAACACCTTATAAA
ATGACAATGCACTTTCAGGAGCCCAGGGCACTGTGGTGAAATGATGATGGCTAGTACAGGTT
ATAAGCCTTGGGGAATTATTTATGAATTCTCAGGATCCTTCAGTTCGCCGCATCCTTCTCCA
TTATTTGAATATTGGAGGCTGCCTGACCAGAATCTTGTCAGGACTTTGCTCCTTCATCCCAG
GTGGTCCCGGCTGACTCCTGAGGACGTTACAGCCCTGAGGGGAGGACTCAGCTTATGAAGTG
CTGGGTGAGACCACTGCCAAGAAGTGCTTGCTCACCCTACCTTCAACGGCAGGGGAATCTCC
CTCTCCTTTTATGGGCGTAGCTGAAGAAAGGATTCATAAATGAAGTTCAATCCTTCTCATCA

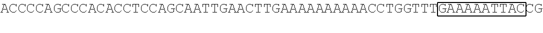

CAAACTATATTGTCATCAAAAAAAAAAAAAAAAAAAACACTTCCTATATTTGAGATGAGAG
AAGAGAGTGCTAGGCA
SEQ ID NO: 17
Cys-Arg-Arg-Arg
SEQ ID NO: 18
Cys-His-His-His
SEQ ID NO: 19
CCTTGCTGCTAAGAGCATCCGCTTGCACCTTCTGCTCATCCCCAGACAAGCTTTGTCCTGTG

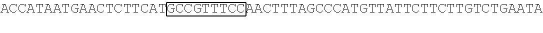

TCCACCCTTTTCTCTGTTCTCAATAATAAGTTCAGGCTTTTCGTCTTCTGAGAAGCCCTTTC
TGACTTCCACAGGCTGAACCACTGGCTTCTGCTCCTCTACATAATACTTCAATTCCAGCATT
GATCTCACTCTATCATGATCATGGGTTTAGCTGTCTGTCCCTGCCACTGCTGTGTGTTCCTC
TTGAGGGCAGGAACATTTGTTTTTCACTTTTTAAAAAACCTCTGTTGCCCAGTCTGGCATTA
GGAAGTGCCCATTAGGTTGTTATTGCTTGTTGGCGCTTGAGCTGGGGCTTGAAGGTTTCTAT
AATGTGTAGCAGTGTATAGAAAACAGGCAGGTCAGAAAAGGCTTCTGTGCATCACACCAACA
TGGCACATGTATACATATGTAACAAATCTGCATGTTGTGCACATGTACCCTAAAACTTAAAG
TATAATAATAATAAAATTTTAAAAAAAAAAAGAAGAGGCTTCCTGGAGGAGATGACAGCTGA
GCTAAGTCCTGGAGGATGAGAAGGAGTATAAAATAAGATAATAGGAGAAAAAAGGCAGTAGG

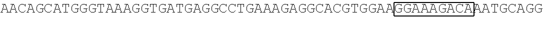

AAGGGGGAATGGGAGGGAATGCTGGGGTACAGGCCAAAGAGGGAGGCATTTGGTGAGTATTC
TGCAGAGTCTCCTCTGCTGTGCTGAGGTGTGGACAATGGGAAACCATGGACGGACTGGAGTA
GGCAAATGTCATATTCCCTGTTACAACTGTCTGTTTGCATGTCAGCCTTCTAGAAGCCCCTT
AAGGTATCAACTATGTTTTTGTTTTGTCATCATTCAATCCTAAGTGCACAGAATTCCGGGCA
TATTACAGGTTCCCCATGAATGTTTCTTTCTTTATTAAAATGTATGAAAACTCTCCAGATTT
AAGGAAGGTCCTCAATGTTTCAAATTCTTTTTGTTAGATCATTGGTCCTGTCTACAGCTGTC
ACAAATTTAAGGACTCTGGTTATATTTAATCTTCACTTTTGAATTTTCTGCTTGAAAAATTT
GTATTAGAAAAAAAAGTCTATCCTTTTATGGACGGCTCTAATCTCTTGAATCATTTGGGTTG
GCTTTTCTTTGGACCTTCTTCAACTCTGTTTTGTCTCTGTTGAGTTAAGGCTTTTAAGAACA

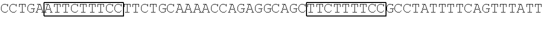

TCTTGTGATTTTAGTTTTTTTCTCTTAACCAAATGCTAAATGGATTTAGGAGAAATAAACTT
ATTTGTAAAGCTGTCAAGGGACCATTAGAAGGATGGTGCTTCACAGATAGAATACAGTTTTT
ATTAATGATGCCTAGACAAATCCTGCCATTAGCCCAAGGGCTCAGAAAGTTAGCAGCCTAGT
AGTTTTGGAGTTGTCAATGAAATGAATTGGACTGGATGGTTAAGGATGCCCAGAAGATTGAA
TAAAATTGGGATTTAGGAGGACCCTTGTACTCCAGGAAATTCTCCAAGTCTCCACTTAGTTA

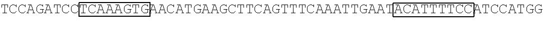

ATTGGCTTGTTTTGTTCAGTTGAGTGCTTGAGGTTGTCTTTTCGACGTAACAGCTAAACCCA
CGGCTTCCTTTCTCGTAAAACCAAAACAAAAAGGCTTTCTATTCAA
SEQ ID NO: 20
AGATGACAGCTGAGCTAAGTCCTGGAGGATGAGAAGGAGTATAAAATAAGATAATAGGAGAA

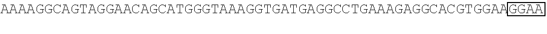

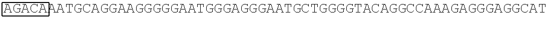

TTGGTGAGTATTCTGCAGAGTCTCCTCTGCTGTGCTGAGGTGTGGACAATGGGAAACCATGG
ACGGACTGGAGTAGGCAAATGTCATATTCCCTGTTACAACTGTCTGTTTGCATGTCAGCCTT
CTAGAAGCCCCTTAAGGTATCAACTATGTTTTTGTTTTGTCATCATTCAATCCTAAGTGCAC
AGAATTCCGGGCATATTACAGGTTCCCCATGAATGTTTCTTTCTTTATTAAAATGTATGAAA
ACTCTCCAGATTTAAGGAAGGTCCTCAATGTTTCAAATTCTTTTTGTTAGATCATTGGTCCT
GTCTACAGCTGTCACAAATTTAAGGACTCTGGTTATATTTAATCTTCACTTTTGAATTTTCT
GCTTGAAAAATTTGTATTAGAAAAAAAAGTCTATCCTTTTATGGACGGCTCTAATCTCTTGA
ATCATTTGGGTTGGCTTTTCTTTGGACCTTCTTCAACTCTGTTTTGTCTCTGTTGAGTTAAG

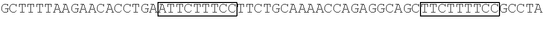

TTTTCAGTTTATTTCTTGTGATTTTAGTTTTTTTCTCTTAACCAAATGCTAAATGGATTTAG
GAGAAATAAACTTATTTGTAAAGCTGTCAAGGGACCATTAGAAGGATGGTGCTTCACAGATA
GAATACAGTTTTTATTAATGATGCCTAGACAAATCCTGCCATTAGCCCAAGGGCTCAGAAAG
TTAGCAGCCTAGTAGTTTTGGAGTTGTCAATGAAATGAATTGGACTGGATGGTTAAGGATGC
CCAGAAGATTGAATAAAATTGGGATTTAGGAGGACCCTTGTACTCCAGGAAATTCTCCAAGT

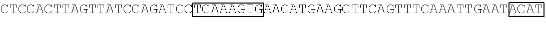

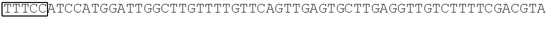

ACAGCTAAACCCACGGCTTCCTTTCTCGTAAAACCAAAACAAAAAGGCTTTCTATTCAA
SEQ ID NO: 21
GTGAGTATTCTGCAGAGTCTCCTCTGCTGTGCTGAGGTGTGGACAATGGGAAACCATGGACG
GACTGGAGTAGGCAAATGTCATATTCCCTGTTACAACTGTCTGTTTGCATGTCAGCCTTCTA
GAAGCCCCTTAAGGTATCAACTATGTTTTTGTTTTGTCATCATTCAATCCTAAGTGCACAGA
ATTCCGGGCATATTACAGGTTCCCCATGAATGTTTCTTTCTTTATTAAAATGTATGAAAACT
CTCCAGATTTAAGGAAGGTCCTCAATGTTTCAAATTCTTTTTGTTAGATCATTGGTCCTGTC
TACAGCTGTCACAAATTTAAGGACTCTGGTTATATTTAATCTTCACTTTTGAATTTTCTGCT
TGAAAAATTTGTATTAGAAAAAAAAGTCTATCCTTTTATGGACGGCTCTAATCTCTTGAATC
ATTTGGGTTGGCTTTTCTTTGGACCTTCTTCAACTCTGTTTTGTCTCTGTTGAGTTAAGGCT

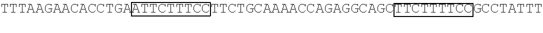

TCAGTTTATTTCTTGTGATTTTAGTTTTTTTCTCTTAACCAAATGCTAAATGGATTTAGGAG
AAATAAACTTATTTGTAAAGCTGTCAAGGGACCATTAGAAGGATGGTGCTTCACAGATAGAA
TACAGTTTTTATTAATGATGCCTAGACAAATCCTGCCATTAGCCCAAGGGCTCAGAAAGTTA
GCAGCCTAGTAGTTTTGGAGTTGTCAATGAAATGAATTGGACTGGATGGTTAAGGATGCCCA
GAAGATTGAATAAAATTGGGATTTAGGAGGACCCTTGTACTCCAGGAAATTCTCCAAGTCTC

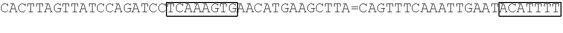

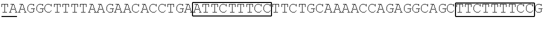

GCTAAACCCACGGCTTCCTTTCTCGTAAAACCAAAACAAAAAGGCTTTCTATTCAA
SEQ ID NO: 22
TCCTGTCTACAGCTGTCACAAATTTAAGGACTCTGGTTATATTTAATCTTCACTTTTGAATT
TTCTGCTTGAAAAATTTGTATTAGAAAAAAAAGTCTATCCTTTTATGGACGGCTCTAATCTC
TTGAATCATTTGGGTTGGCTTTTCTTTGGACCTTCTTCAACTCTGTTTTGTCTCTGTTGAGT

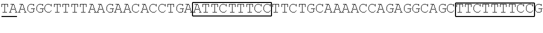

CCTATTTTCAGTTTATTTCTTGTGATTTTAGTTTTTTTCTCTTAACCAAATGCTAAATGGAT
TTAGGAGAAATAAACTTATTTGTAAAGCTGTCAAGGGACCATTAGAAGGATGGTGCTTCACA
GATAGAATACAGTTTTTATTAATGATGCCTAGACAAATCCTGCCATTAGCCCAAGGGCTCAG
AAAGTTAGCAGCCTAGTAGTTTTGGAGTTGTCAATGAAATGAATTGGACTGGATGGTTAAGG
ATGCCCAGAAGATTGAATAAAATTGGGATTTAGGAGGACCCTTGTACTCCAGGAAATTCTCC

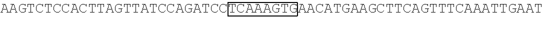

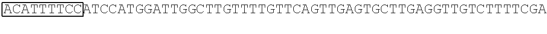

CGTAACAGCTAAACCCACGGCTTCCTTTCTCGTAAAACCAAAACAAAAAGGCTTTCTATTCA
A
SEQ ID NO: 23
TAGACAAATCCTGCCATTAGCCCAAGGGCTCAGAAAGTTAGCAGCCTAGTAGTTTTGGAGTT
GTCAATGAAATGAATTGGACTGGATGGTTAAGGATGCCCAGAAGATTGAATAAAATTGGGAT

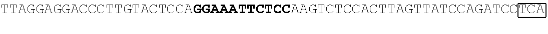

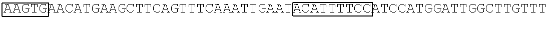

TGTTCAGTTGAGTGCTTGAGGTTGTCTTTTCGACGTAACAGCTAAACCCACGGCTTCCTTTC
TCGTAAAACCAAAACAAAAAGGCTTTCTATTCAA
SEQ ID NO: 24
GTATTTTTCTCTGTGCAACAGCCCTGAGCTCTAGAACTTATGACAGCTTTTCTCAAGTGAAG

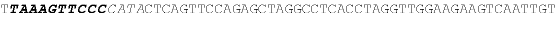

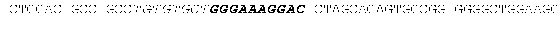

CTTTCCTCATGATGCTACGGGGAGTACAGTGAAAAGGGAGGAGATTGGGGTAGCAAGGTCCC

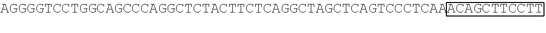

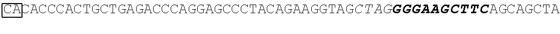

GGGGGATCCCTGCAGGATCAGCCTCCTCCTTACCTTGCAGACCCCTTGGCTGTGGTCTGGCA
TTCCTAGTGGCTGGCCTAAAGCCCCAAACCTGGAATCTGTTTCAGAGCCTGGACCTTTTGGA

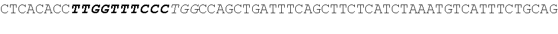

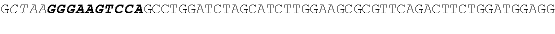

GATCAGCAGGCCTTGGTATTGGTGGTTCTACCTGCTTCGGTTAAGAGTGAGGACTCTGGAGC
CAGACTCTCTAAATCATAGCACTGACACTTACTATATGACTTGGGCCAGGTTCGTTACCTGT
CTGTGCCTCAGTTTTCCTCATCTGTAAAATGGGAGATTAGTGGTCACCATTGGGATGATTAA

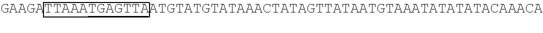

GTAACCATTTACATGCATATAAAACATAATATAAATGTAATATTAGCTATATTCATATATAA
AAATAGGTATATTTTATATATAAAATATATTACATTTAATTTTTTACATTTTGCATTAAGAT
ATATTATTTCATATAAAACATTTAAGTACATATACATATAATTATCTATGTAATTATTATTG
TTATTGTTGTTATTGTCTACTTTACCTCCCTGCAGGGATGGGGAGACCACGGCAGGCTTTTA
AACATTCTTGGTCTCACTGAGCCGTAGGTGAGGAATCCCTGGTTCCTACAGACTCTGCTGTA

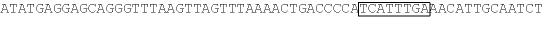

ACAATGAATGCCACATAAATATCATTTCTCAGATTCCTATGATGCTCTTCTTTCAGATCTTT

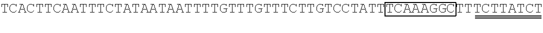

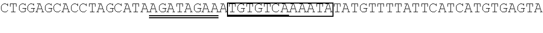

TTTTTAGGTCCTGTTAACCCCCATAACTATTGATTCAGAGAAGTAGGGTGGTTCTGAAAAAT

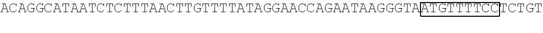

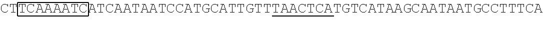

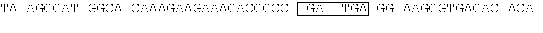

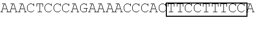

SEQ ID NO: 25

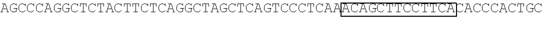

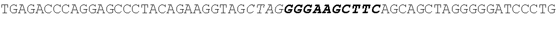

CAGGATCAGCCTCCTCCTTACCTTGCAGACCCCTTGGCTGTGGTCTGGCATTCCTAGTGGCT

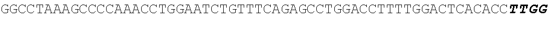

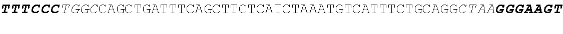

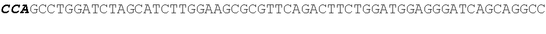

TTGGTATTGGTGGTTCTACCTGCTTCGGTTAAGAGTGAGGACTCTGGAGCCAGACTCTCTAA
ATCATAGCACTGACACTTACTATATGACTTGGGCCAGGTTCGTTACCTGTCTGTGCCTCAGT

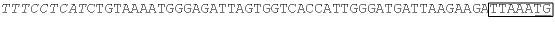

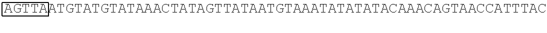

ATGCATATAAAACATAATATAAATGTAATATTAGCTATATTCATATATAAAAATAGGTATAT
TTTATATATAAAATATATTACATTTAATTTTTTACATTTTGCATTAAGATATATTATTTCAT
ATAAAACATTTAAGTACATATACATATAATTATCTATGTAATTATTATTGTTATTGTTGTTA
TTGTCTACTTTACCTCCCTGCAGGGATGGGGAGACCACGGCAGGCTTTTAAACATTCTTGGT
CTCACTGAGCCGTAGGTGAGGAATCCCTGGTTCCTACAGACTCTGCTGTAATATGAGGAGCA

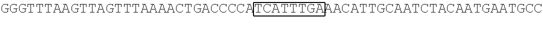

ACATAAATATCATTTCTCAGATTCCTATGATGCTCTTCTTTCAGATCTTTTCACTTCAATTT

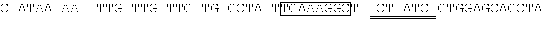

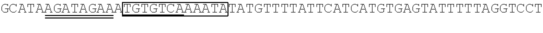

GTTAACCCCCATAACTATTGATTCAGAGAAGTAGGGTGGTTCTGAAAAATACAGGCATAATC

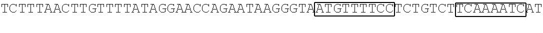

CAATAATCCATGCATTGTTTAACTCATGTCATAAGCAATAATGCCTTTCATATAGCCATTGG

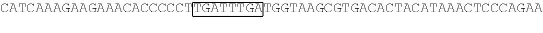

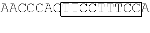

SEQ ID NO: 26

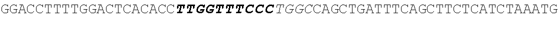

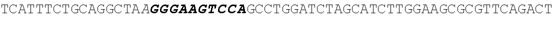

TCTGGATGGAGGGATCAGCAGGCCTTGGTATTGGTGGTTCTACCTGCTTCGGTTAAGAGTGA
GGACTCTGGAGCCAGACTCTCTAAATCATAGCACTGACACTTACTATATGACTTGGGCCAGG
TTCGTTACCTGTCTGTGCCTCAGTTTTCCTCATCTGTAAAATGGGAGATTAGTGGTCACCAT

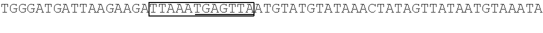

TATATACAAACAGTAACCATTTACATGCATATAAAACATAATATAAATGTAATATTAGCTAT
ATTCATATATAAAAATAGGTATATTTTATATATAAAATATATTACATTTAATTTTTTACATT
TTGCATTAAGATATATTATTTCATATAAAACATTTAAGTACATATACATATAATTATCTATG
TAATTATTATTGTTATTGTTGTTATTGTCTACTTTACCTCCCTGCAGGGATGGGGAGACCAC
GGCAGGCTTTTAAACATTCTTGGTCTCACTGAGCCGTAGGTGAGGAATCCCTGGTTCCTACA

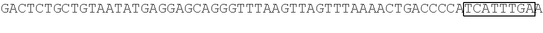

ACATTGCAATCTACAATGAATGCCACATAAATATCATTTCTCAGATTCCTATGATGCTCTTC

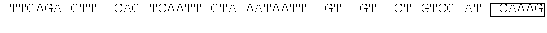

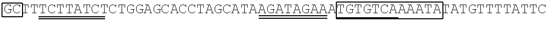

ATCATGTGAGTATTTTTAGGTCCTGTTAACCCCCATAACTATTGATTCAGAGAAGTAGGGTG

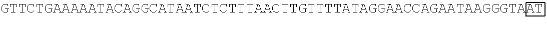

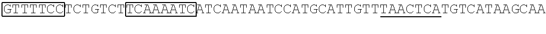

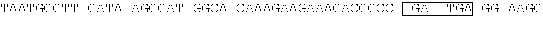

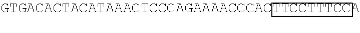

SEQ ID NO: 27
CTGACACTTACTATATGACTTGGGCCAGGTTCGTTACCTGTCTGTGCCTCAGTTTTCCTCAT

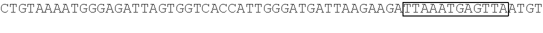

ATGTATAAACTATAGTTATAATGTAAATATATATACAAACAGTAACCATTTACATGCATATA
AAACATAATATAAATGTAATATTAGCTATATTCATATATAAAAATAGGTATATTTTATATAT
AAAATATATTACATTTAATTTTTTACATTTTGCATTAAGATATATTATTTCATATAAAACAT
TTAAGTACATATACATATAATTATCTATGTAATTATTATTGTTATTGTTGTTATTGTCTACT
TTACCTCCCTGCAGGGATGGGGAGACCACGGCAGGCTTTTAAACATTCTTGGTCTCACTGAG
CCGTAGGTGAGGAATCCCTGGTTCCTACAGACTCTGCTGTAATATGAGGAGCAGGGTTTAAG

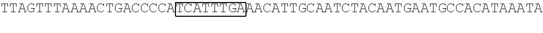

TCATTTCTCAGATTCCTATGATGCTCTTCTTTCAGATCTTTTCACTTCAATTTCTATAATAA

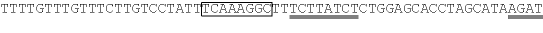

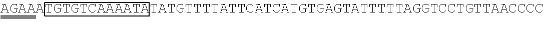

CATAACTATTGATTCAGAGAAGTAGGGTGGTTCTGAAAAATACAGGCATAATCTCTTTAACT

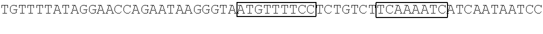

ATGCATTGTTTAACTCATGTCATAAGCAATAATGCCTTTCATATAGCCATTGGCATCAAAGA

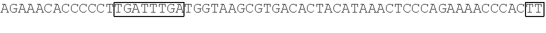

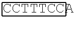

SEQ ID NO: 28
CAGTAACCATTTACATGCATATAAAACATAATATAAATGTAATATTAGCTATATTCATATAT
AAAAATAGGTATATTTTATATATAAAATATATTACATTTAATTTTTTACATTTTGCATTAAG
ATATATTATTTCATATAAAACATTTAAGTACATATACATATAATTATCTATGTAATTATTAT
TGTTATTGTTGTTATTGTCTACTTTACCTCCCTGCAGGGATGGGGAGACCACGGCAGGCTTT
TAAACATTCTTGGTCTCACTGAGCCGTAGGTGAGGAATCCCTGGTTCCTACAGACTCTGCTG

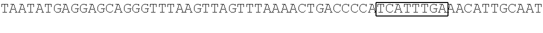

CTACAATGAATGCCACATAAATATCATTTCTCAGATTCCTATGATGCTCTTCTTTCAGATCT

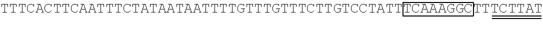

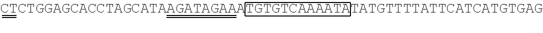

TATTTTTAGGTCCTGTTAACCCCCATAACTATTGATTCAGAGAAGTAGGGTGGTTCTGAAAA

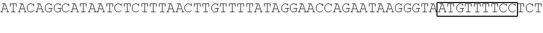

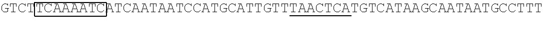

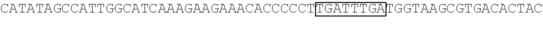

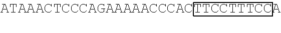

SEQ ID NO: 29
CCTGGTTCCTACAGACTCTGCTGTAATATGAGGAGCAGGGTTTAAGTTAGTTTAAAACTGAC

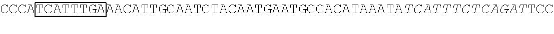

TATGATGCTCTTCTTTCAGATCTTTTCACTTCAATTTCTATAATAATTTTGTTTGTTTCTTG

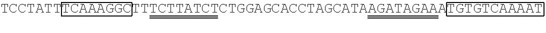

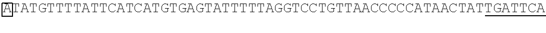

GAGAAGTAGGGTGGTTCTGAAAAATACAGGCATAATCTCTTTAACTTGTTTTATAGGAACCA

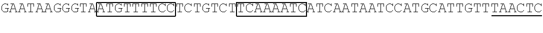

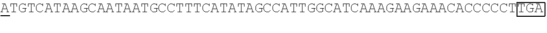

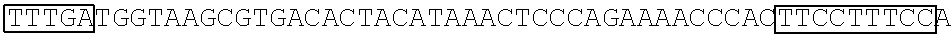

As used herein, “consisting essentially of” allows the inclusion of materials or steps that do not materially affect the basic and novel characteristics of the claim. Any recitation herein of the term “comprising”, particularly in a description of components of a composition or in a description of elements of a device, can be exchanged with the alternative expressions “consisting essentially of” or “consisting of”.

While the present invention has been described in conjunction with certain preferred embodiments, one of ordinary skill, after reading the foregoing specification, will be able to effect various changes, substitutions of equivalents, and other alterations to the compositions and methods set forth herein.

### REFERENCES

1. Blood. 2014 Apr. 24; 123(17):e68-78
2. Molecular and cellular biology 1997, p. 4220-4229
3. Science 7 Nov. 2003: Vol. 302, Issue 5647, pp. 1041-1043
4. J Immunol. 2015 Oct. 1; 195(7):3058-70
5. J Immunol. 1996 156:4154-4159
6. J Biol. Chem. 2006 Sep. 29; Vol. 281, No 39, pp. 28666-28678
7. European J Immunol. 2012. 42:1850-1862

1. A promoter sequence for use in expression of a transgene under control of the promoter sequence in a CD3+ cell, the promoter sequence comprising nucleotides 1501-2000 of any of SEQ ID NOS:2-10 or 12-16, or a variant thereof having at least 90% identity to said sequence.

2. The promoter sequence of claim 1, wherein the promoter sequence comprises nucleotides 1001-2000 of any one of SEQ ID NOS:2-10 or 12-16 or a variant thereof having at least 90% identity to said sequence.

3. The promoter sequence of claim 2, wherein the promoter sequence comprises nucleotides 501-2000 of any one of SEQ ID NOS:2-10 or 12-16 or a variant thereof having at least 90% identity to said sequence.

4. The promoter sequence of claim 3, wherein the promoter sequence comprises the nucleotide sequence of any one of SEQ ID NOS:2-16 or a variant thereof having at least 90% identity to said sequence.

5. The promoter sequence of any of claims 1-4, wherein the promoter sequence comprises a binding sequence for one or more transcription factors selected from the group consisting of NF-kappaB, AP-1, STAT, GATA-3, and NFAT.

6. The promoter sequence of any of claims 1-5, wherein the promoter is capable of expressing the transgene at a higher level in CD3+ cells compared to CD3− cells.

7. The promoter sequence of claim 6, wherein the ratio of expression in CD3+ cells to CD3− cells is at least 2:1.

8. A promoter sequence for use in expression of a transgene under control of the promoter sequence in a CD3+ cell, the promoter sequence comprising SEQ ID NO: 2, SEQ ID NO:3, SEQ ID NO:7, SEQ ID NO:11, or SEQ ID NO:13.

9. A vector, plasmid, or nucleic acid molecule comprising the promoter sequence of any of claims 1-8.

10. The vector of claim 9 which is a viral vector.

11. The viral vector of claim 10 which is a retrovirus, a lentivirus, an adenovirus, an adeno-associated virus, or a herpes simplex virus.

12. The viral vector of any of claims 9-11 which is incorporated into a nanoparticle.

13. The viral vector of any of claims 9-11 which is not incorporated into a nanoparticle.

14. The viral vector of any of claims 9-11 whose envelope lacks a fusion protein.

15. The vector of any of claims 9-14, wherein the vector comprises a transgene encoding a product selected from the group consisting of chimeric antigen receptors (CARs), checkpoint inhibitors, cytokines, chemokines, antibodies and antigen binding fragments and variants thereof, enzymes, structural proteins, and reporter genes.

16. The nucleic acid molecule of claim 9 which is an RNA molecule.

17. A cell comprising the vector, plasmid, or nucleic acid molecule of any of claims 9-16.

18. The cell of claim 17, wherein the cell comprises a genome-integrated viral vector.

19. The cell of claim 17, wherein the cell comprises an episomal form of the vector.

20. A nanoparticle comprising the vector of any of claims 9-15, wherein the nanoparticle is capable of delivery of the vector into a CD3+ cell.

21. The nanoparticle of claim 20, wherein the nanoparticle comprises a targeting moiety that promotes selective entry of the nanoparticle into CD3+ cells.

22. The nanoparticle of claim 20 or 21, wherein the nanoparticle is also capable of delivery of the vector into a CD3− cell.

23. The nanoparticle of any of claims 20-22, wherein the nanoparticle comprises a polymer.

24. The nanoparticle of claim 23, wherein the polymer is a poly(beta-amino ester).

25. A method of expressing a transgene in a CD3+ cell, the method comprising the steps of:
(a) providing the vector of any of claims 9-15, or the nanoparticle of any of claims 20-24, and a CD3+ cell, wherein the vector comprises said transgene;
(b) transducing or transfecting the cell with the vector; and
(c) allowing the transgene to be expressed in the transduced or transfected cell.

26. The method of claim 25, wherein the vector is a lentiviral vector.

27. The method of claim 25, wherein the CD3+ cell is CD4+, CD4−, CD8+, or CD8−.

28. The method of claim 25, wherein step (b) comprises contacting the vector with a mixture of CD3+ and CD3− cells, and wherein the CD3+ cells are selectively transduced.

29. The method of any of claims 25-28, wherein step (b) is performed in vitro.

30. The method of any of claims 25-28, wherein step (b) is performed in vivo and comprises administration of the vector by intravenous, intratumoral, intramedullary, or intraperitoneal injection.

31. A method of making the vector of any one of claims 9-15, the method comprising adding the promoter sequence of any of claims 1-8 to a vector for use in transducing a CD3+ cell.

32. The method of claim 31, wherein said promoter sequence does not support expression of the transgene in packaging cells or producer cells used to make the vector.
